(19) United States
(12) Patent Application Publication
(10) Pub. No.: US 2006/0204992 A1
(43) Pub. Date: Sep. 14, 2006

(54) Method for determining hair cycle markers

(76) Inventors: Olaf Holtkotter, Hurth (DE); Dirk Petersohn, Koln (DE); Kordula Schlotmann, Dusseldorf (DE); Melanie Giesen, Geldern (DE); Danlala Kessler-Becker, Leverkusen (DE)

Correspondence Address: DANN DORFMAN HERRELL AND SKILLMAN;A PROFESSIONAL CORPORATION, 1601 MARKET STREET, SUITE 2400, PHILADELPHIA, PA, 19103-2307, US

(21) Appl. No.: 11/364,118

(22) Filed: Feb. 28, 2006

Related U.S. Application Data
(63) Continuation of application No. PCT/EP04/09435, filed on Jul. 24, 2004.

(30) Foreign Application Priority Data
Aug. 30, 2003 (DE) 103 40 373.6

(51) Int. Cl. C12Q 1/68

(52) U.S. Cl. 435/6

(57) ABSTRACT

The invention relates to a method for determining hair cycle markers in vitro, test kits and biochips which are used to determine hair cycle markers and to the use of proteins, mRNA molecules, proteins or fragments thereof as hair cycle markers. The invention also relates to a test method which is used to detect the effectiveness of cosmetic and pharmaceutical active ingredients which influence the hair cycle, in addition to a screening method which is used to identify cosmetic or pharmaceutical active ingredients which influence the hair cycle and to a method for the production of a cosmetic and pharmaceutical preparation which influences the hair cycle.

### CROSS REFERENCE TO RELATED APPLICATIONS

This application is a §365 (c) continuation application of PCT/EP2004/009435 filed 24 Jul. 2004, which in turn claims priority to DE application 103 40 373.6 filed 30 Aug. 2003. Each of the foregoing applications is incorporated herein by reference.

### FIELD OF THE INVENTION

This invention relates to a process for determining hair cycle markers in vitro, to test kits and biochips for determining hair cycle markers and to the use of proteins, mRNA molecules or fragments of proteins or mRNA molecules as hair cycle markers; to a test method for demonstrating the effectiveness of cosmetic or pharmaceutical active substances for influencing the hair cycle and to a screening process for identifying cosmetic or pharmaceutical active substances for influencing the hair cycle and to a process for the production of a cosmetic or pharmaceutical preparation for influencing the hair cycle.

### BACKGROUND OF THE INVENTION

Besides its actual biological function, the hair has a psychosocial function which is not to be underestimated. Unwanted hair loss or excessive hair growth can have a serious negative impact on the self-consciousness of the person affected (Paschier et al. (1988), Int. J. Dermatol. 27: 441-446). Except for rare congenital hair diseases caused by mutations in keratins or other structural proteins, excessive hair loss and excessive hair growth are caused by a disturbed hair cycle. Hair follicles pass through a cycle of three stages: anagen (growth phase), catagen (regression phase) and telogen (resting phase). Androgenic alopecia is characterized, for example, by an increasingly shorter anagen phase coupled with a reduction in size of the hair follicle (see, for example, Paus and Cotsarelis (1999), New Eng. J. Med., 341: 491-497).

Assigning the hair follicle to a stage of the hair cycle is essentially done on the basis of a microscopic-morphological analysis of the hair. Knowledge of the molecular mechanisms which play a role in the progression through the hair cycle is only fragmentary. Consequently, molecular markers characteristic of a certain stage of the hair follicle are lacking as are molecular targets through which the state of the hair follicle can be influenced. Although a number of different markers of hair-covered human skin were identified in DE 102 60931 to Applicants, those markers are characteristic of the anagenic hair follicles which make up most of the hair-covered skin.

The inadequate number of markers characteristic of other stages of the hair cycle leads to deficiencies in the general description of the growth phases of the hair in vivo, in cultivated hair follicles in vitro (Philpott Model; Philpott M. et al. (1990). Human Hair Growth in vitro; J. Cell Sci. 97: 463-471, 1990) and in reconstructed hair follicle models. In the latter systems in particular, morphological classification in stages of the hair cycle is no longer readily possible. Hair follicles cultivated in vitro are evaluated by microscopic measurement of the growth in length with a measuring ocular, including photographic documentation, and by histological evaluation of complicated vertical sections. This form of analysis is very time-consuming and requires a large number of hair follicles to cover the individual variations. For evaluating reconstructed hair follicle models, characterization via molecular markers of the corresponding stage is crucially important.

Besides the ratio of proliferation to apoptosis in the follicles, the DNA/protein and keratin synthesis and the ATP content, markers for the growth phase of hair follicles have hitherto been purely individual markers, for example matrix proteins, such as collagen type IV, fibronectin and laminin (Couchman, J. R. et al. (1985), Dev. Biol. 108: 290-298), growth factors, such as Transforming Growth Factor TGF-β1 and TGF-β2 (Foitzik et al. (2000), FSEB, J. 14: 752-760; Tsutomu, S. et al. (2002), J. Invest. Dermatol. 118: 993-997) and Fibroblast Growth Factor FGF-7 (Herbert, J. M. et al. (1994), Cell 78: 1017-1025). However, problems have arisen from the fact that many of these markers resulted from studies of the synchronized hair cycle of mice and cannot readily be applied to the human hair cycle.

In addition, the fragmentary knowledge of the molecular mechanisms playing a role in the progression through the hair cycle leads to an inadequate number of targets which are available for cosmetically or pharmacologically influencing the hair follicles. Thus, the enzyme 5α-reductase (type II) is the only validated target for androgenic alopecia. Inhibition of this enzyme, for example by the active principle finasteride, results in a reduced concentration of dihydrotestosterone in the skin and in the serum and hence in inhibition of the androgen-dependent miniaturization of the hair follicles. The disadvantage of finasteride undoubtedly lies in the side effects associated with its use: pregnant women in particular should not use finasteride. In addition, finasteride may not be used in cosmetic formulations.

The analysis of molecular markers in hair follicles is complicated as only relatively small quantities of mRNA can be obtained from the follicles and the concentration of such mRNA molecules is quite low, e.g., only a few to several hundred copies per cell in the hair follicles. Weakly expressed genes have only been accessible to existing analysis techniques with great difficulty, if at all, but can play a crucial role in the hair follicle.

There has never been a description of the transcriptome, i.e. the totality of all transcribed genes, of the hair follicles in various stages of the cell cycle.

Transcriptome analyses of the skin by various processes, including SAGE™ analysis, are already known. However, they are conducted with isolated keratinocytes (in vitro) or epidermis explantates which, as explained above, are not models representative of the complex events in the skin.

It is known from applicants' DE-A-101 00 127.4-41 that skin cells can be subjected to SAGE™ analysis in order to characterize the overall transcriptome of the skin. Applicants' DE-A-101 00 121.5-41 discloses the identification of markers of stressed or aged skin on the basis of a comparative SAGE™ analysis between stressed or aged skin and unstressed or young skin. However, there is no information on specific hair cycle markers in either of these documents.

It is known from J. Invest. Dermatol. 2002 July; 119(1): 3-13; “A serial analysis of gene expression in sun-damaged human skin”; Urschitz, J. et al., that markers of sun-damaged skin can be determined by a comparative SAGE™ analysis of whole skin explantates taken from in front of the auricle (sun-damaged) and behind the auricle (protected from the sun). Knowledge of specific hair cycle markers cannot be acquired from this publication either.

Accordingly, a need exists for the identification of genes which are markers important to the hair cycle.

### SUMMARY OF THE INVENTION

In accordance with the present invention, a large number of the genes important to the hair cycle have been identified thereby enabling further genetic characterization of hair cycle regulation and screening processes for identifying active substances for influencing the hair cycle.

In one aspect, an in vitro method for determining hair cycle phase in humans is provided. An exemplary method entails providing a plurality of genetically encoded markers isolated from hair covered human skin or from human hair follicles which are differentially expressed at the anagenic phase of the hair cycle when compared to expression in cells in the catagenic phase of the hair cycle. A sample of hair covered skin or human hair follicles is obtained and analyzed for the presence and optionally the quantity of at least one genetically encoded molecule which is differentially expressed in anagenic and catagenic hair follicles. The sample is then designated as comprising healthy cells in the anagenic phase of the cycle if it contains markers which are expressed at higher levels in anagenic hair follicles or cells in regression in the catagenic phase of the hair cycle if it contains molecules which are expressed at higher levels in catagenic hair follicles. The genetically encoded markers encompassed by the foregoing method comprise at least one mRNA molecule, at least one protein or polypeptide or fragments thereof.

Tables 2 to 9 provide a plurality of markers that are differentially expressed in anagenic phase of the hair cycle when compared to the catagenic phase of the hair cycle. Such markers can be used to advantage in the methods of the present invention.

In another embodiment of the invention, the expression levels of at least two molecules in the sample which are differentially expressed in cells from the anagenic phase of the hair cycle when compared to expression levels in the catagenic phase of the hair cycle are quantified and the expression ratios of the at least two molecules determined thereby forming an expression quotient. The expression ratios obtained are compared with those in column 5 of Tables 2 to 6 and the sample designated as healthy cells in the anagenic phase of the hair cycle if the expression ratios observed in the follicles correspond to the ratios observed in anagenic hair follicles or cells in regression in the catagenic phase of the hair cycle if the expression ratios correspond to those observed in catagenic hair follicles.

Also encompassed by the present invention is a test kit for determining hair cycle phase in a human subject. An exemplary test kit comprises reagents suitable for performing the method described above. Thus, a kit of the invention comprises a plurality of probes corresponding to those provided in Tables 2-9 which are optionally detectably labelled, a solid support such as a biochip and physiological buffers for assessing gene expression levels. The kit may also comprise means for obtaining genetically encoded molecules or markers from hairy skin or hair follicles.

Thus, in yet another aspect of the invention, a biochip for determining hair cycle phase in human beings in vitro is provided comprising a solid, i.e. rigid or flexible, carrier and a plurality of probes immobilized thereon which are capable of specifically binding to at least one molecule selected from the group consisting of SEQ ID NO:1 to SEQ ID NO: 570 or the corresponding gene product. SEQ ID NOS:1-570 represent markers for determining hair cycle phase in human beings in vitro Exemplary markers are selected from the group consisting of at least one molecule having a Swissprot Accession Number provided in column 8 of Table 8, a Swissprot Accession Number provided in column 9 of Table 7, a Swissprot Accession Number provided in column 9 of Table 9, a UniGene Accession Number provided in column 7 of Tables 2 to 6, and a Swissprot Accession Number in column 8 of Tables 2 to 6.

Also provided in the present invention is an in vitro method for identifying a pharmaceutically active agent which modulates the hair cycle. An exemplary method entails providing hair covered human skin or human follicles comprising cells; determining the phase of the hair cycle of said cells as described above; contacting the cells with the agent at least once; and repeating the determination of the phase of the hair cycle to determine whether said agent alters the phase of the hair cycle. In a preferred embodiment, the method is performed on a biochip. A test kit for performing the method described above is also provided herein. Finally, a pharmaceutical preparation comprising the agent identified in the foregoing screening method having efficacy against diseases or impairment of hair and its growth in a pharmaceutically acceptable carrier is also disclosed.

Diseases or disorders of the hair cycle include, for example pili torti (corkscrew hair, twisted hair), monilethrix (spindle hair), woolly hair (kinked hair), hair shaft defects with breakages [Trichorrhexis nodosa, Trichorrhexis invaginata, Trichoschisis, trichoptilosis (split hair shafts)], hair shaft defects through metabolic disorders, pili recurvati, rolled hair, changes in hair color [heterochromy, albinism, poliosis (acquired patch-like absence of pigment in the hair), canitis (physiological graying)], hypertrichoses, hirsutism, alopecias (irreversible alopecia: for example, androgenetic alopecia in men and women); reversible alopecia: for example symptomatic diffuse alopecias through infections, chem. noxas and medicaments, hormonal disorders, diseases, etc.) and alopecia greata.

### DETAILED DESCRIPTION OF THE INVENTION

The totality of all the mRNA molecules synthesized at a certain time by a cell or a tissue is known as a transcriptome. The technique of serial analysis of gene expression (SAGE™) (Velculescu, V. E. et al., 1995, Science 270, 484-487) is used for understanding the transcriptome of human hair follicles. This technique facilitates the simultaneous identification and quantitation of the genes expressed in hair follicles. Comparison of the transcriptome of anagenic hair follicles with the transcriptome of catagenic hair follicles identifies those genes which are important for these stages of the hair cycle. These may be genes which are highly expressed in anagenic hair follicles or conversely, genes which are only weakly expressed when compared to expression levels observed in catagenic hair follicles.

Although gene expression can also be analyzed by the quantitation of specific mRNA molecules (for example Northern Blot, and/or RNase protection experiments), only a limited number of genes can be measured by these techniques. Theoretically, SAGE™ analysis could be replaced by MPSS (massive parallel signature sequencing) or by techniques based on differential display. In practice, however, the SAGE™ technique is faster and more reliable than alternative methods and is therefore preferred.

The SAGE method is based on two principles. First, only a short nucleotide sequence from the 3′ region of the mRNA is required for identification of the gene. A sequence of nine base pairs allows the differentiation of 262,144 (4⁹) transcripts. This is more than the number of all the genes present in the genome. Second, concatenation of the short sequences allows efficient automated analysis by sequencing. An advantage of this technique not to be underestimated is the ability to determine the reading direction of the genes. If two opposite transcripts of a gene in the reading direction are started, this can only detected by the SAGE technique.

Typically, double-stranded cDNA is synthesized with biotinylated primers from polyA-RNA. The cDNA is digested with a restriction enzyme (anchoring enzyme) recognizing 4 bp which statistically cuts all 256 bp. The 3′ end of the cDNA is isolated by binding to Streptavidin beads. The sample is divided into two halves and the cDNA end is ligated with a linker (1 or 2) which has a recognition site for a type IIS restriction enzyme (tagging enzyme). This cuts up to 20 bp staggered from the asymmetric recognition site. This results in the formation of a short sequence (tag) tied to the linker which is unique to each gene. In order to obtain relatively large quantities of material, the linker1 tags are ligated with the linker2 tags after the projecting ends have been filled (linker ditag). The ligation products are amplified with linker-specific primers (1 or 2). The linker no longer in use is then released by another enzymatic digestion with the anchoring enzyme. The isolated ditags are concatenated by ligation (concatemers), cloned in a vector and transfixed in cells. From the cells, the concatemers are amplified via PCR and, finally, sequenced.

Another promising method is the microarray or chip technique. Here, entire gene libraries are placed on a chip. The genes on the chip are hybridized with fluorescence-marked cDNA generated from the mRNA of the tissue sample to be analyzed. By comparing anagenic with catagenic follicle material, all interesting genes can be detected in a single test on the basis of the differences in fluorescence. However, this does presuppose a knowledge of the clones in the gene library.

A very advantageous analysis method is the combination of SAGE analysis with the microarray technique. The SAGE method provides new or known genes which can be meaningful to the hair cycle. These are projected onto a chip with which samples of individual candidates can be measured.

Human hair follicles from healthy female donors were used for the SAGE™ analysis. The follicles were isolated from pieces of tissue taken from above the ear of the donor and were divided on the basis of their morphology into catagenic and anagenic hair follicles. In order to minimize the detection of donor-specific variances, the catagenic and anagenic hair follicles of a total of five donors were combined. The same number of catagenic and anagenic follicles of a donor were used and the total number of follicles of the individual donors were assimilated to one another.

The SAGE™ analysis was carried out as described in Velculescu, V. E. et al., 1995 Science 270, 484-487. A SAGE™ bank for catagenic hair follicles and one for anagenic hair follicles were analyzed. For further analysis, the two SAGE™ banks were standardized to the mean tag count. The two banks were compared with one another in order to identify genes demonstrating hair-cycle-specific regulation. As expected for two banks of the same tissue type, the tag repertoire of the two follicle banks is largely similar. Despite the similarity of the tissue and the relatively small number of tags, 197 tags show a differential expression with a significance of p>0.05. The significance was determined as described in Audic, S., Clayerie, J. M. (1997): “The significance of digital gene expression profiles”, Genome Res. 7: 986-95.

Table 1 lists markers for which a differential expression as a function of the stage of the hair cycle has already been described. They serve as positive controls for the experiment. Table 1 shows
the relative expression frequency in anagenic hair follicles in column 1,
the relative expression frequency in catagenic hair follicles in column 2,
the quotient of the relative expression frequency determined in anagenic hair follicles and the relative expression frequency determined in catagenic hair follicles in column 3,
the significance of the values shown in column 3 in column 4,
the UniGene Accession Number in column 5
the Swissprot Accession Number in column 6 and
the name of the gene from which the corresponding tag originates in column 7.

The quotient in column 3 indicates the strength of the differential expression, i.e. the factor by which the particular gene is expressed more strongly in anagenic hair follicles than in catagenic hair follicles or vice versa.

The particular genes or gene products for Tables 1-6 are disclosed under their UniGene Accession Number in the data bank of the National Center for Biotechnology Information (NCBI). This data bank is accessible on the world wide web at ncbi/nim.nih.gov. In addition, the genes or gene products are directly accessible at the following world wide web addresses ncbi.nlm.nih.gov/UniGene/Hs.Home.html or ncbl.nlm.nih.gov/genome/guide.

Mice comprising inactivated vitamin D receptor demonstrate hair loss. It was shown that, after stimulation of the anagen stage by shaving, mice with an inactive vitamin D receptor are unable to initiate the hair cycle (Kong et al. (1002), J. Invest. Dermatol., 118: 631-8).

Thrombospondin-1 was shown to play a role in the induction of hair follicle involution and in vascular degradation during the catagen phase (Yano et al. (2003), J. Invest. Dermatol., 120: 14-9). Whereas no expression of the thrombospondin can be detected in the early to middle anagen phase, high expression levels can be detected during the catagenic phase in accordance with the expression data found there.

Although the role of neurotrophin-5 for human hair follicles has never been described, studies of the family member neurotrophin-3 in murine hair follicles have been conducted. Maximal expression of neutrotrophin was observed in the catagenic stage (Botchkarev et al. (1998), Am. J. Pathol., 153: 785-99). A corresponding expression pattern was found there for neurotrophin-5.

In the course of SAGE™, the number of individual tags was determined in a first step and, where possible, assigned to genes or inputs in the UniGene data bank. By comparison of the tags in the various SAGE™ banks, differentially expressed genes can be identified. Accordingly, a first classification was made based on the significance of the differential expression of the identified genes as genes which are significantly differentially expressed are considered marker genes for particular stages of the hair cycle.

The genes for which a significant differential expression was found are listed in Tables 2 to 6.

Tables 2 to 6 contain a detailed list of the genes differentially expressed in anagenic hair follicles and in catagenic hair follicles, as determined by the process according to the invention, with an indication of
the running sequence identifier (SEQ ID NO:) in column 1,
the tag sequence used in column 2,
the relative expression frequency in anagenic hair follicles in column 3,
the relative expression frequency in catagenic hair follicles in column 4,
the quotient of the relative expression frequency determined in anagenic hair follicles and the relative expression frequency determined in catagenic hair follicles in column 5,
the significance of the values shown in column 5 in column 6,
the UniGene Accession Number in column 7,
the Swissprot Accession Number in column 8 and
a brief description of the gene or gene product in column 9.

The quotient in column 5 indicates the strength of the differential expression, i.e. the factor by which the particular gene is expressed more strongly in anagenic hair follicles than in catagenic hair follicles or vice versa.

Table 2 lists all the genes which exhibit at least five-fold differential expression levels in anagenic hair follicles when compared to levels observed in catagenic hair follicles with a p value of p<0.01 (significance>2.0).

Table 3 lists all the genes which exhibit at least two-fold differential expression levels in anagenic hair follicles when compared to those observed in catagenic hair follicles with a p value of p<0.01 (significance>2.0).

Table 4 lists all the genes which exhibit at least 1.3 fold differential expression levels in anagenic hair follicles when compared to those observed in catagenic hair follicles with a p value of p<0.01 (significance>2.0).

Table 5 lists all the genes which exhibit at least five-fold differential expression levels in anagenic hair follicles when compared to levels observed in catagenic hair follicles with a p value of p<0.05 (significance>1.3).

Table 6 lists all the genes which exhibit at least two-fold differential expression levels in anagenic hair follicles when compared to those observed in catagenic hair follicles with a p value of p<0.05 (significance>1.3).

The clear expression difference in the ribosomal RNAs is particularly noticeable. Slight expression differences in ribosomal RNAs have hitherto been described as typical artefacts of SAGE™. In the present case, however, the expression differences are strikingly high and uniform. There is a much stronger expression of rRNA in anagenic hair follicles than in catagenic hair follicles. Accordingly, the strength of expression of ribosomal RNA is itself a marker criterion for anagenic hair follicles.

In addition, there are some other biologically interesting expression differences. First, the expression of attractin in catagenic hair follicles is increased. Attractin is a protein from the agouti/melanocortin signal transduction pathway. The gene product plays a role in determining the hair color of mice (Gunn et al. (1999), Nature, 398: 152-6; Barsh et al. (2002), J. Recept. Signal Transduct. Res., 22: 63-77).

In addition, cobalamin adenosyl transferase, an enzyme in the vitamin B12 metabolism pathway, is induced in catagenic hair follicles. In human beings, a vitamin B12 deficiency leads to depigmentation of the hair (Mori et al. (2001), J. Dermatotol. 28: 282-5). Dopachrome tautomerase, an enzyme involved in the biosynthesis of melanin, is also induced in catagenic hair follicles. All the genes mentioned above are relevant to hair follicle biology, particularly to pigmentation, but have not hitherto been described in connection with regulation of the hair cycle.

It is also noticeable that the transcription factors Fos-B and Egr1 are induced in catagenic hair follicles. These two transcription factors belong to the group of so-called immediate-early genes and have wide-reaching regulatory functions.

On the other hand, the angiopoietin-like protein CDT6 is repressed in catagenic hair follicles. This protein is assumed to have a regulatory function in angiogenesis (Peek et al. (2002), J. Biol. Chem., 277: 686-93). Control of angiogenesis and hence the supply of blood to the hair follicle is coupled to the hair cycle (see above, thrombospondin-1).

Also noteworthy is the induction of the 14-3-3 sigma protein, stratifin, and the simultaneous repression of the 14-3-3 tau/theta protein. The family of 14-3-3 proteins regulate a number of enzymes, including those involved in primary metabolism and the cell cycle. They also have a chaperone function. They can activate the transcription of inducible genes and regulate signal transduction and apoptosis processes. A role in the differentiation of keratinocytes was described in particular for the 14-3-3 sigma protein, stratifin (Dellambra et al. (1995), J. Cell Sci. 108:3569-79). A specific regulation of the members of this protein family in the various hair follicle stages is therefore extremely likely. Finally, keratin 6A and acidic hair keratin are also repressed in catagenic hair follicles.

Any evaluation of whether or not the differential expression of various genes is significant is critically determined by the number of sequenced tags. Non-significant expression differences can become statistically significant through an increase in the number of sequenced tags.

The relevance of subsignificant expression differences can be evaluated using various data analysis methods through which expert biological knowledge flows into the evaluation of the expression differences. One method is the clustering of the identified genes according to their GO annotation. The GO annotation derives from the inputs in the data bank of the Gene Ontology (GO) Consortium, in which individual genes/proteins are classified according to their (primary) function. See world wide website geneontology.org/. By using these relationship features, expression differences which are statistically not outside the confidence interval can also assume a significance.

Table 7 contains a detailed list of the genes differentially expressed in anagenic hair follicles and in catagenic hair follicles, as determined by the process according to the invention, with an indication of
the running sequence identifier (SEQ ID NO:) in column 1,
the tag sequence used in column 2,
the relative expression frequency in anagenic hair follicles in column 3,
the relative expression frequency in catagenic hair follicles in column 4,
the ratio of the relative expression frequency determined in anagenic hair follicles and the relative expression frequency determined in catagenic hair follicles to one another in column 5,
the significance of the values shown in column 5 in column 6,
the GO number in column 7,
a brief description of the gene or gene product in column 8 and
the Swissprot Accession Number in column 9

The quotient in column 5 indicates the strength of the differential expression, i.e. the factor by which the particular gene is expressed more strongly in anagenic hair follicles than in catagenic hair follicles or vice versa.

The particular genes or gene products are accessible on the internet under their GO number at the following world wide web address geneontology.org.

For example genes of the DPP-IV cluster, a family of dipeptidyl peptidases (attractin [anagen 8 tags: catagen 23 tags], DPP-9 [0:9], DPP-4 [0:2], DPP-8 [0:1]), are clearly induced in catagenic hair follicles. The dipeptidyl peptidases of the DPP-IV family are proline-specific proteases which function to regulate various pathological and physiological processes (Aleski and Malik (2001), Biochim. Biophys. Act, 1550: 107-116). In addition, there is a weak, but consistent induction of various DNA repair helicases, for example RecQ-like 5 [3:8], RecQ-like 4 [1:2], RuvB-like [0:3], etc. This induction can be found in all annotated helicases of this set of data. In addition, the melanin biosynthesis cluster, which includes inter alia dopachrome tautomerase [0:7] and silver/pMEL [7:17], is also clearly induced.

By contrast, various subunits of type IV collagen (α1 [5:1], α2 [1:0], α6 [4:0]) are induced in anagenic hair follicles. Type IV collagen is a typical constituent of the follicle matrix and the expression of this protein can be expected to be increased in the growth phase of the follicle. The synaptosome cluster is also induced in anagenic hair follicles. This cluster includes the SNARE proteins VAMP-2 [5:0] and VAMP-3 [4:0] which have a general role in secretion. This observation is supported by the general induction of genes which play a role in exocytosis. This induction of exocytosis genes is likely associated with the process of pigmentation of the hair. Pigmentation involves the transfer of melanin-synthesizing organelles, so-called melanosomes, from melanocytes to keratinocytes of the hair follicle. Melanosomes bear a large microscopic similarity to the synaptosomes of the nerve cells, secretory vesicles which enable neurotransmitters to be released. The role of SNARE proteins for the synaptosomes is sufficiently documented; the role of these proteins in melanosomes is under discussion at the present time (Scott et al. (2002); J. Cell. Sci., 115: 1441-51). Finally, genes belonging to the group with N-acetyl lactosamine synthase activity (chain 1 [3:0], chain 2 [8:2], chain 3 [1:0]) are induced in anagenic hair follicles. Poly-N-acetyl lactosamine structures are found both in N- and in O-linked glycans of the glycoproteins from mammals. These glycans presumably interact with selectins and other glycan-binding proteins (Zhou (2003), Curr. Protein Pept. Sci., 4:1-9).

Another method of increasing the relevance of subsignificantly differentially expressed genes is clustering according to sequence patterns. Such clustering is possible by co-ordinating the SAGE data with the data from available domain and pattern data banks, for example PROSITE and Pfam at world wide web site sanger.ac.uk/Software/Pfam/index.shtml and espasy.ch/prosite/.

Table 8 contains a detailed list of the genes differentially expressed in anagenic hair follicles and in catagenic hair follicles, as determined by the process according to the invention, with an indication of
the running sequence identifier (SEQ ID NO:) in column 1,
the tag sequence used in column 2,
the relative expression frequency in anagenic hair follicles in column 3,
the relative expression frequency in catagenic hair follicles in column 4,
the ratio of the relative expression frequency determined in anagenic hair follicles and the relative expression frequency determined in catagenic hair follicles to one another in column 5,
the significance of the values shown in column 5 in column 6,
a brief description of the pattern or the gene or gene product in column 7 and
the Swissprot Accession Number in column 8.

The quotient in column 5 indicates the strength of the differential expression, i.e. the factor by which the particular gene is expressed more strongly in anagenic hair follicles than in catagenic hair follicles or vice versa.

Through this co-ordination, the significance of some already described genes is further increased. Thus, the GO cluster with dipeptidyl peptidase activity is extended by other members of the PF:PEPTIDASE_S9 family. In addition, proteins with a GRAM domain are clearly induced in the catagenic hair follicles. The function of the domain is not known at present (Doerks et al. (2000) Trends Biochem. Sci., 25: 483-485).

As already described for GO clusters, type IV collagen subunits (C4 domain) are repressed in catagenic hair follicles in this arrangement also. The induction of proteins with a Gla domain in the anagenic hair follicles is noteworthy. These proteins are matrix-Gla and osteocalcin proteins. The matrix-Gla protein was described as an BMP-2 antagonist in hair follicle development and in the cycle (Nakamura et al. (2003), FASEB J., 17: 497-9).

In addition, the significance of differential gene expression can be increased by lexical analysis. In this case, a search is made for corresponding keywords in the descriptive texts of the various genes, as found for example in the data bank annotations.

Table 9 contains a detailed list of the genes differentially expressed in anagenic hair follicles and in catagenic hair follicles, as determined by the process according to the invention, with an indication of
the running sequence identifier (SEQ ID NO:) in column 1,
the tag sequence used in column 2,
the relative expression frequency in anagenic hair follicles in column 3,
the relative expression frequency in catagenic hair follicles in column 4,
the ratio of the relative expression frequency determined in anagenic hair follicles and the relative expression frequency determined in catagenic hair follicles to one another in column 5,
the significance of the values shown in column 5 in column 6,
the target word in column 7,
a brief description of the gene or gene product in column 8 and
the Swissprot Accession Number in column 9.
   The quotient in column 5 indicates the strength of the differential expression, i.e. the factor by which the particular gene is expressed more strongly in anagenic hair follicles than in catagenic hair follicles or vice versa.

As a result of this analysis, catagenic hair follicles show a significant induction of the cluster with the keyword “autophagy” (Apg4 [2:7], Apg3 [0:2], Apg10 [0:2], Apg5 [0:1]. Autophagy is a process in which cells envelop macroscopic cell constituents, such as organelles for example, in autophagosomes and then digest them in the lysosome. Autophagy occurs primarily during cell supply deficiencies; excessive autophagy is regarded as a mechanism of non-apoptotic programmed cell death. In addition, clusters formed on the basis of the keywords “dsc2” and “desmocollin” are repressed in catagenic hair follicles. Localization in the hair follicle has been reported in particular for desmocollin-3 (Kurzen et al. (1998), Differentiation, 63: 295-304; Nuber et al. (1996), Eur. J. Cell Biol., 71: 1-13).

Previously, it had been demonstrated that ribosomal RNA expression was repressed in catagenic hair follicles. These data are confirmed by the analytic methods described herein.

Finally, the repression of selenoproteins in catagenic hair follicles is also striking.

In yet another aspect of the invention a process (2) for determining the hair cycle in human beings, more particularly in women, in vitro, is provided. An exemplary method entails

a) obtaining a mixture of proteins, mRNA molecules or fragments of either from hair-covered human skin or from human hair follicles,

b) analyzing the mixture of a) for the presence and optionally the quantity of at least one of the proteins, mRNA molecules or fragments of either which are differentially expressed in anagenic and catagenic human hair follicles as shown by (SAGE),

c) comparing the analysis results from b) with the expression patterns identified by serial analysis of gene expression (SAGE) and

d) assigning the mixture to growing or healthy hair if it predominantly contains proteins, mRNA molecules or fragments of either which demonstrate elevated expression levels in anagenic hair follicles when compared to those observed in catagenic hair follicles or to hair in regression or unhealthy hair if it predominantly contains proteins, mRNA molecules or fragments of proteins or mRNA molecules which demonstrate elevated expression in catagenic hair follicles than in anagenic hair follicles.

Diseases or disorders of the hair cycle include, for example pili torti (corkscrew hair, twisted hair), monilethrix (spindle hair), woolly hair (kinked hair), hair shaft defects with breakages [Trichorrhexis nodosa, Trichorrhexis invaginata, Trichoschisis, trichoptilosis (split hair shafts)], hair shaft defects through metabolic disorders, pili recurvati, rolled hair, changes in hair color [heterochromy, albinism, poliosis (acquired patch-like absence of pigment in the hair), canitis (physiological graying)], hypertrichoses, hirsutism, alopecias (irreversible alopecia: for example, androgenetic alopecia in men and women); reversible alopecia: (for example symptomatic diffuse alopecias through infections, chem. noxas and medicaments, hormonal disorders, diseases, etc.) and alopecia greata.

The mixture obtained in step a) above may be obtained from whole skin samples, hair-covered skin equivalents, isolated hair follicles, hair follicle equivalents or cells of hair-covered skin.

It may be sufficient in step b) to analyze the mixture obtained for the presence of at least one of the proteins, mRNA molecules or fragments of either which are identified by serial analysis of gene expression (SAGE) as differentially expressed in anagenic and catagenic hair follicles where they are expressed solely in anagenic hair follicles or solely in catagenic hair follicles. In other cases, the quantity of the differentially expressed molecules must also be determined in step b), i.e. the expression must be quantitated.

In step d), the mixture analyzed in step b) is assigned to growing or healthy hair if it predominantly contains proteins, mRNA molecules or fragments of proteins or mRNA molecules which demonstrate elevated expression levels in anagenic hair follicles when compared to those observed in catagenic hair follicles, i.e. the mixture contains either more different compounds typically expressed in anagenic hair follicles than those which are typically expressed in catagenic hair follicles (qualitative differentiation) or more copies of compounds typically expressed in anagenic hair follicles than are typically present in catagenic hair follicles (quantitative differentiation). For assignment to hair in regression or unhealthy hair, the complementary procedure is followed.

A preferred embodiment of the method of the invention for determining the hair cycle is characterized in that, in step b), the mixture obtained is analyzed for the presence and optionally the quantity of at least one of the proteins, mRNA molecules or fragments of either which are identified by their Swissprot Accession Number in column 9 of Table 9 and, in step d), the mixture analyzed in b) is assigned to growing or healthy hair if it predominantly contains proteins, mRNA molecules or fragments of proteins or mRNA molecules which are expressed more strongly in anagenic hair follicles than in catagenic hair follicles or the mixture analyzed in b) is assigned to hair in regression or unhealthy hair if it predominantly contains proteins, mRNA molecules or fragments of proteins or mRNA molecules which are expressed more strongly in catagenic hair follicles than in anagenic hair follicles.

Another preferred embodiment of the method of the invention for determining the hair cycle is characterized in that, in step b), the mixture obtained is analyzed for the presence and optionally the quantity of at least one of the proteins, mRNA molecules or fragments of either which are identified by their Swissprot Accession Number in column 8 of Table 8 and, in step d), the mixture analyzed in b) is assigned to growing or healthy hair if it predominantly contains proteins, mRNA molecules or fragments of either which are expressed more strongly in anagenic hair follicles than in catagenic hair follicles or the mixture analyzed in b) is assigned to hair in regression or unhealthy hair if it predominantly contains proteins, mRNA molecules or fragments of proteins or mRNA molecules which are expressed more strongly in catagenic hair follicles than in anagenic hair follicles.

Another preferred embodiment of the process according to the invention for determining the hair cycle is characterized in that, in step b), the mixture obtained is analyzed for the presence and optionally the quantity of at least one of the proteins, mRNA molecules or fragments of proteins or mRNA molecules which are identified by their Swissprot Accession Number in column 9 of Table 7 and, in step d), the mixture analyzed in b) is assigned to growing or healthy hair if it predominantly contains proteins, mRNA molecules or fragments of proteins or mRNA molecules which are expressed more strongly in anagenic hair follicles than in catagenic hair follicles or the mixture analyzed in b) is assigned to hair in regression or unhealthy hair if it predominantly contains proteins, mRNA molecules or fragments of either which are expressed more strongly in catagenic hair follicles than in anagenic hair follicles.

Another preferred embodiment of the process according to the invention for determining the hair cycle is characterized in that, in step b), the mixture obtained is analyzed for the presence and optionally the quantity of at least one of the proteins, mRNA molecules or fragments of either which are identified by their Unigene Accession Number in column 7 of Table 6, by their Swissprot Accession Number in column 8 or by the brief description of the gene or gene product in column 9 and, in step d), the mixture analyzed in b) is assigned to growing or healthy hair if it predominantly contains proteins, mRNA molecules or fragments of either which are expressed at least twice as strongly in anagenic hair follicles as in catagenic hair follicles or the mixture analyzed in b) is assigned to hair in regression or unhealthy hair if it predominantly contains proteins, mRNA molecules or fragments of proteins or mRNA molecules which are expressed at least twice as strongly in catagenic hair follicles as in anagenic hair follicles.

Another preferred embodiment of the method according to the invention for determining the hair cycle is characterized in that, in step b), the mixture obtained is analyzed for the presence and optionally the quantity of at least one of the proteins, mRNA molecules or fragments of either which are identified by their Unigene Accession Number in column 7 of Table 5, by their Swissprot Accession Number in column 8 or by the brief description of the gene or gene product in column 9 and, in step d), the mixture analyzed in b) is assigned to growing or healthy hair if it predominantly contains proteins, mRNA molecules or fragments of proteins or mRNA molecules which are expressed at least five times as strongly in anagenic hair follicles as in catagenic hair follicles or the mixture analyzed in b) is assigned to hair in regression or unhealthy hair if it predominantly contains proteins, mRNA molecules or fragments of proteins or mRNA molecules which are expressed at least five times as strongly in catagenic hair follicles as in anagenic hair follicles.

Another particularly preferred embodiment of the method according to the invention for determining the hair cycle is characterized in that, in step b), the mixture obtained is analyzed for the presence and optionally the quantity of at least one of the proteins, mRNA molecules or fragments of either which are identified by their Unigene Accession Number in column 7 of Table 4, by their Swissprot Accession Number in column 8 or by the brief description of the gene or gene product in column 9 and, in step d), the mixture analyzed in b) is assigned to growing or healthy hair if it predominantly contains proteins, mRNA molecules or fragments thereof which are expressed at least 1.3 times as strongly in anagenic hair follicles as in catagenic hair follicles or the mixture analyzed in b) is assigned to hair in regression or unhealthy hair if it predominantly contains proteins, mRNA molecules or fragments of proteins or mRNA molecules which are expressed at least 1.3 times as strongly in catagenic hair follicles as in anagenic hair follicles.

Another particularly preferred embodiment of the method according to the invention for determining the hair cycle is characterized in that, in step b), the mixture obtained is analyzed for the presence and optionally the quantity of at least one of the proteins, mRNA molecules or fragments of either which are identified by their Unigene Accession Number in column 7 of Table 3, by their Swissprot Accession Number in column 8 or by the brief description of the gene or gene product in column 9 and, in step d), the mixture analyzed in b) is assigned to growing or healthy hair if it predominantly contains proteins, mRNA molecules or fragments of proteins or mRNA molecules which are expressed at least twice as strongly in anagenic hair follicles as in catagenic hair follicles or the mixture analyzed in b) is assigned to hair in regression or unhealthy hair if it predominantly contains proteins, mRNA molecules or fragments of proteins or mRNA molecules which are expressed at least twice as strongly in catagenic hair follicles as in anagenic hair follicles.

Another most particularly preferred embodiment of the method according to the invention for determining the hair cycle is characterized in that, in step b), the mixture obtained is analyzed for the presence and optionally the quantity of at least one of the proteins, mRNA molecules or fragments of either which are identified by their Unigene Accession Number in column 7 of Table 2, by their Swissprot Accession Number in column 8 or by the brief description of the gene or gene product in column 9 and, in step d), the mixture analyzed in b) is assigned to growing or healthy hair if it predominantly contains proteins, mRNA molecules or fragments of either which are expressed at least five times as strongly in anagenic hair follicles as in catagenic hair follicles or the mixture analyzed in b) is assigned to hair in regression or unhealthy hair if it predominantly contains proteins, mRNA molecules or fragments of proteins or mRNA molecules which are expressed at least five times as strongly in catagenic hair follicles as in anagenic hair follicles.

The hair cycle can also be described by quantitating several markers (expression products of the genes of importance to anagenic or catagenic hair follicles) which then have to be active in a characteristic ratio to one another in order to represent healthy or growing hair or in a different characteristic ratio to one another in order to represent hair in regression or unhealthy hair.

Accordingly, the present invention also relates to a method (3) for determining the hair cycle in human beings, more particularly in women, in vitro. An exemplary method entails
a) obtaining a mixture of proteins, mRNA molecules or fragments of either from hair-covered human skin or from human hair follicles, b) quantitating the expression levels of at least two of the proteins, mRNA molecules or fragments of either previously identified by SAGE as modulators of the hair cycle,
c) determining the expression ratios of the at least two proteins, mRNA molecules or fragments of either and forming an expression quotient,
d) comparing the expression ratios from c) with the expression ratios typically present in anagenic or in catagenic hair follicles for the molecules quantitated in b), more particularly with the expression ratios listed in column 5 of Tables 2 to 6 and
e) assigning the mixture obtained in a) to growing or healthy hair if the expression ratios of the follicles investigated or the hair-covered skin investigated correspond to the expression ratios in anagenic hair follicles or the mixture obtained in a) is assigned to hair in regression or unhealthy hair if the expression ratios of the follicles investigated or the hair-covered skin investigated correspond to the expression ratios in catagenic hair follicles.

The mixture obtained in step a) of the method according to the invention is preferably obtained from a skin sample, more particularly from a whole skin sample.

In another embodiment of the method according to the invention, the mixture obtained in step a) is obtained by microdialysis. The technique of microdialysis is described, for example, in “Microdialysis: A method for measurement of local tissue metabolism”, Nielsen, P. S., Winge, K., Petersen, L. M.; Ugeskr Laeger 1999, Mar. 22 161:12 1735-8; and in “Cutaneous microdialysis for human in vivo dermal absorption studies”, Anderson, C. et al.; Drugs Pharm. Sci., 1998, 91, 231-244; and also on the internet at world wide web address microdialysis.se/technique.htm, which is incorporated by reference herein.

In the technique of microdialysis, a probe is typically inserted into the skin and then slowly rinsed with a suitable carrier solution. After the acute reactions have abated following the insertion, the microdialysis yields proteins, mRNA molecules or fragments thereof which are present in the extracellular space and which can then be isolated in vitro, for example by fractionation of the carrier liquid, and analyzed. Microdialysis is less invasive than removing a whole skin sample, but has the disadvantage that it is limited to obtaining molecules occurring in the extracellular space.

Another preferred embodiment of the process according to the invention is characterized in that, in step b) of process (2), the analysis for the presence and optionally the quantity of at least one of the proteins or protein fragments or, in process (3), the quantitation of at least two proteins or protein fragments is carried out by a method selected from
one- or two-dimensional gel electrophoresis
affinity chromatography
protein-protein complexing in solution
mass spectrometry, more particularly matrix assisted laser desorption ionization (MALDI) and, more particularly,
use of protein chips or by a suitable combination of these methods.

Suitable analytical methods for use in the invention are described in the overview article by Akhilesh Pandey and Matthias Mann: “Proteomics to study genes and genomes”, Nature, Volume 405, Number 6788, 837-846 (2000), and the references cited therein, which is incorporated herein by reference.

2D gel electrophoresis is described, for example, in L. D. Adams, “Two-dimensional gel electrophoresis using the Isodalt System” or in L. D. Adams and S. R. Gallagher, Two-dimensional Gel Electrophoresis using the O'Farrell System”; both in Current Protocols in Molecular Biology (1997, Eds. F. M. Ausubel et al.), Unit 10.3.1-10.4.13; or in 2D Electrophoresis Manual; T. Berkelman, T. Senstedt; Amersham Pharmacia Biotech, 1998 (Order No. 80-6429-60).

The mass-spectrometric characterization of the proteins or protein fragments is carried out in methods known to those of skill in the art, for example as described in the following literature references:
Methods in Molecular Biology, 1999; Vol. 112; 2-D Proteome Analysis Protocols; Editor: A. J. Link; Humana Press; Totowa, N.J., more particularly Courchesne, P. L. and Patterson, S. D.; pp. 487-512.
Carr, S. A. and Annan, R. S.; 1997; in Current Protocols in Molecular Biology; Editor: Ausubel, F. M. et al.; John Wiley and Sons, Inc. 10.2.1-10.21.27.

Another preferred embodiment of the process according to the invention is characterized in that, in step b) of process (2), the analysis for the presence and optionally the quantity of at least one of the mRNA molecules or mRNA molecule fragments or, in process (3), the quantitation of at least two mRNA molecules or mRNA molecule fragments is carried out by a method selected from
Northern blots,
reverse transcriptase polymerase chain reaction (RT-PCR),
Rnase protection experiments,
dot blots,
cDNA sequencing,
clone hybridization,
differential display,
subtractive hybridization,
cDNA fragment fingerprinting,
total gene expression analysis (TOGA),
serial analysis of gene expression (SAGE)
massively parallel signature sequencing (MPSS®) and, more particularly use of nucleic acid chips or by suitable combinations of these methods.

These methods are suitable for use in the invention and are described in the overview articles by Akhilesh Pandey and Matthias Mann: “Proteomics to study genes and genomes”, Nature, Volume 405, Number 6788, 837-846 (2000), and “Genomics, gene expression and DNA arrays”, Nature, Volume 405, Number 6788, 827-836 (2000) and the references cited therein, which are incorporated by reference herein. The TOGA process is described in J. Gregor Sutcliffe et al. “TOGA: An automated parsing technology for analyzing expression of nearly all genes, Proceedings of the National Academy of Sciences of the United States of America (PNAS), Vol. 97, No. 5, pp. 1976-1981 (2000)”, which is also incorporated herein by reference. The MPSS® process is described in U.S. Pat. No. 6,013,445, which is also incorporated herein by reference.

According to the invention, however, other methods known to the skilled person for analyzing for the presence and optionally the quantity of at least one of the proteins, mRNA molecules or fragments thereof may also be used.

Another preferred embodiment of the process according to the invention is characterized in that step b) comprises analyzing for the presence and optionally the quantity of 1 to ca. 5,000, preferably 1 to ca, 1,000, more particularly ca. 10 to ca. 500, preferably ca. 10 to ca. 250, more preferably ca. 10 to ca. 100 and most preferably ca. 10 to ca. 50 of the proteins, mRNA molecules or fragments thereof which are defined by their Swissprot Accession Number in column 8 of Table 8, by their Swissprot Accession Number in column 9 of Tables 7 and 9 and by their UniGene Accession Number in column 7 of Tables 2 to 6, by their Swissprot Accession Number in column 8 or by the brief description of the gene or gene product in column 9.

The present invention also relates to a test kit for determining the hair cycle in human beings in vitro comprising means for carrying out the process according to the invention for determining the hair cycle in human beings.

The present invention also relates to a biochip for determining the hair cycle in human beings in vitro comprising
a solid, i.e. rigid or flexible, carrier and
probes immobilized thereon which are capable of specifically binding to at least one of the proteins, mRNA molecules or fragments of proteins or mRNA molecules defined by their Swissprot Accession Number in column 8 of Table 8, by their Swissprot Accession Number in column 9 of Tables 7 and 9 and by their UniGene Accession Number in column 7 of Tables 2 to 6, by their Swissprot Accession Number in column 8 or by the brief description of the gene or gene product in column 9.

A biochip is a miniaturized functional element with molecules, more particularly biomolecules, which can act as specific interaction partners immobilized on one surface. The structure of these functional elements often comprises rows and columns which are known as chip arrays. Since thousands of biological or biochemical functional elements can be accommodated on one chip, they generally have to be made by microtechnical methods.

Biological and biochemical functional elements include, in particular, DNA, RNA, PNA (in the case of nucleic acids and their chemical derivatives, single strands, triplex structures or combinations thereof, for example, may be present), saccharides, peptides, proteins (for example antibodies, antigens, receptors) and derivatives of combinatorial chemistry (for example organic molecules).

Biochips generally have a 2D base surface for coating with biologically or biochemically functional materials. The base surfaces may also be formed, for example, by walls of one or more capillaries or by channels.

The prior art is represented, for example, by the following publications: Nature Genetics, Vol. 21, Supplement (whole), January 1999 (biochips); Nature Biotechnology, Vol. 16, pp. 981-983, October 1998 (biochips); Trends in Biotechnology, Vol. 16, pp. 301-306, July 1998 (biochips) and the above-cited overview articles by Akhilesh Pandey and Matthias Mann: Proteomics to study genes and genomes”, Nature, Volume 405, Number 6788, 837-846 (2000), and “Genomics, gene expression and DNA arrays”, Nature, Volume 405, Number 6788, 827,836 (2000), and the literature cited therein, which are all incorporated herein by reference.

A clear account of processes for the practical application of DNA chip technology is presented in the books “DNA Microarrays: A Practical Approach” (Editor: Mark Schena, 1999, Oxford University Press) and “Microarray Biochip Technology” (Editor: Mark Schena, 2000, Eaton Publishing), to the whole of which reference is hereby made.

DNA chip technology which is based on the ability of nucleic acid to enter into complementary base pairing is particularly preferred for the purposes of the present invention. This technical principle, known as hybridization, has already been used for years in Southern blot and Northern blot analysis. By comparison with these conventional methods, in which only a few genes are analyzed, DNA chip technology enables a few hundred to several thousand genes to be analyzed simultaneously.

A DNA chip consists essentially of a carrier material (for example glass or plastic) on which single-stranded, gene-specific probes are immobilized in high densities in a particular place (spot). The technique of probe application and the chemistry of probe immobilization are regarded as problematic. At present, there are several ways of achieving probe immobilization. E. M. Southern (E. M. Southern et al. (1992), Nucleic Acid Research 20, 1679-1684 and E. M. Southern et al. (1997), Nucleic Acid Research 25, 1155-1161) describes the production of oligonucleotide arrangements by direct synthesis on a glass surface which had been treated with 3-glycidoxypropyl trimethoxysilane and then with a glycol. A similar process achieves the in situ synthesis of oligonucleotides by a photosensitive combinatorial chemistry which can be compared with photolithographic techniques (Pease, A. C. et al. (1994), Proc. Natl. Acad Sci USA 91, 5022-5026).

Besides these techniques based on the in situ synthesis of oligonucleotides, already existing DNA molecules can also be immobilized on surfaces of carrier material. P.O. Brown (DeRisi et al. (1997), Science 278, 680-686) describes the immobilization of DNA on glass surfaces coated with polylysine. An article by L. M. Smith (Guo, Z. et al. (1994), Nucleic Acid Research 22, 5456-5465) discloses a similar process: oligonucleotides bearing a 5′-terminal amino group can be immobilized on a glass surface which had been treated with 3-aminopropyl trimethoxysilane and then with 1,4-phenyl diisothiocyanate.

DNA probes can be applied to a carrier with a so-called pin spotter. To this end, thin metal needles, for example 250 μm in diameter, dip into probe solutions and then transfer the adhering sample material in defined volumes to the carrier material of the DNA chip.

However, the probes are preferably applied by a piezo-controlled nanodispenser which, similarly to an ink jet printer, applies probe solutions contactlessly to the surface of the carrier material in a volume of 100 picoliters.

The probes are immobilized, for example, as described in EP-A-0 965 647. DNA probes are generated by PCR using a sequence-specific primer pair, one primer being modified at the 5′-end and carrying a linker with a free amino group. This ensures that a defined strand of the PCR products can be immobilized on a glass surface which had been treated with 3-aminopropyl trimethoxysilane and then with 1,4-phenyl diisothiocyanate. The gene-specific PCR products should ideally have a defined nucleic acid sequence in a length of 200 to 400 bp and comprise non-redundant sequences. After the immobilization of the PCR products via the derivatized primer, the counter-strand of the PCR product is removed by incubation for 10 minutes at 96° C.

In one application typical of DNA chips, mRNA is isolated from two cell populations to be compared. The isolated mRNAs are converted into cDNA by reverse transcription using fluorescence-marked nucleotides for example. The samples to be compared are marked, for example, with red or green fluorescing nucleotides. The cDNAs are then hybridized with the gene probes immobilized on the DNA chip and the immobilized fluorescent signals are then quantitated.

A factor critical to the success of using DNA chip technology for analyzing the gene expression of the hair follicles is the composition of the gene-specific probes on the DNA chip. The relevant genes of the hair cycle as identified in SAGE™ analysis are particularly useful in this regard. Since extremely small quantities of mRNA occasionally have to be analyzed where a DNA chip is used for analyzing the relevant hair cycle genes, it may be necessary to enrich the mRNA before the analysis by means of so-called linear amplification (Zhao et al. (2002), BMC Genomics, 3:31). To this end, the mRNA of a sample is first transcribed into cDNA. The amplified RNA is obtained from this double-stranded cDNA by in vitro transcription.

The analysis chips mentioned in DE-A-100 28 257.1-52 and in DE-A-101 02 063.5-52 are most particularly preferred for the production of small biochips (containing up to ca. 500 probes). These analysis chips have an electrically addressable structure which enables the samples to be electrofocused. In this way, samples can advantageously be focused and immobilized irrespective of their viscosity at particular points of an array by means of electrodes. The focusing ability simultaneously provides for an increase in the local concentration of the samples and thus for higher specificity. During the analysis itself, the test material can be addressed at the individual positions of the array. Thus, each item of information analyzed can potentially be tracked with the highest possible sensitivity. Cross-contamination by adjacent spots is virtually impossible.

The biochip according to the invention comprises 1 to ca. 5,000, preferably 1 to ca. 1,000, more particularly ca. 10 to ca. 500, preferably ca. 10 to ca. 250, more preferably ca. 10 to ca. 100 and most preferably ca. 10 to ca. 50 different probes. The different probes can each be present on the chip in multiple copies.

The biochip according to the invention comprises nucleic acid probes, more particularly RNA or PNA probes and preferably DNA probes. The nucleic acid probes have a length of ca. 10 to ca. 1,000 nucleotides, preferably ca. 10 to ca. 800 nucleotides, more preferably ca. 100 to ca. 600 nucleotides and most preferably ca. 200 to ca. 400 nucleotides.

A particularly preferred biochip according to the invention is a DNA chip carrying probes selected from those listed in Tables 2 and 5 and in Table 3 (without mitochondrial and ribosomal tags) and the over-represented groups “DNA helicase activity”, “DPPIV activity” and “melanine biosynthesis from tyrosine” from Table 7.

In another preferred form, the biochip according to the invention comprises peptide or protein probes, more particularly antibodies.

The present invention also relates to the use of the proteins, mRNA molecules or fragments of proteins or mRNA molecules which are defined by their Swissprot Accession Number in column 8 of Table 8, by their Swissprot Accession Number in column 9 of Tables 7 and 9 and by their UniGene Accession Number in column 7 of Tables 2 to 6, by their Swissprot Accession Number in column 8 and by the brief description of the gene or gene product in column 9 as hair cycle markers in human beings.

The present invention also relates to a test method for demonstrating the effectiveness of cosmetic or pharmaceutical active principles for influencing the hair cycle, more particularly against diseases or impairment of the hair and its growth, in vitro, characterized in that

a) the hair status is determined by a process according to the invention for determining the hair cycle or by means of a test kit according to the invention for determining the hair cycle or by means of a biochip according to the invention,

b) an active principle against diseases or impairment of the hair and its growth is applied one or more times to the hair-covered skin,

c) the hair status is re-determined by a process according to the invention for determining the hair cycle or by means of a test kit according to the invention for determining the hair cycle or by means of a biochip according to the invention and

d) the effectiveness of the active principle is determined by comparison of the results from a) and c).

The test method according to the invention can be carried out with whole skin samples, hair-covered skin equivalents, isolated hair follicles, hair follicle equivalents or cells of hair-covered skin.

The present invention also relates to a test kit for demonstrating the effectiveness of cosmetic or pharmaceutical active principles against diseases or impairment of the hair and its growth, comprising means for carrying out the test method according to the invention.

The present invention also relates to the use of the proteins, mRNA molecules or fragments of proteins or mRNA molecules which are defined by their Swissprot Accession Number in column 8 of Table 8, by their Swissprot Accession Number in column 9 of Tables 7 and 9 and by their UniGene Accession Number in column 7 of Tables 2 to 6, by their Swissprot Accession Number in column 8 or by the brief description of the gene or gene product in column 9 for demonstrating the effectiveness of cosmetic or pharmaceutical active principles against diseases or impairment of the hair and its growth.

The present invention also relates to a screening process for identifying cosmetic or pharmaceutical active principles against diseases or impairment of the hair and its growth in vitro, characterized in that

a) the hair status is determined by a process according to the invention for determining the hair cycle or by means of a test kit according to the invention for determining the hair cycle or by means of a biochip according to the invention,

b) a potential active principle against diseases or impairment of the hair and its growth is applied one or more times to the hair-covered skin,

c) the hair status is re-determined by a process according to the invention for determining the hair cycle or by means of a test kit according to the invention for determining the hair cycle or by means of a biochip according to the invention and

d) effective active principles are determined by comparison of the results from a) and c).

The present invention also relates to the use of the proteins, mRNA molecules or fragments of proteins or mRNA molecules which are defined by their Swissprot Accession Number in column 8 of Table 8, by their Swissprot Accession Number in column 9 of Tables 7 and 9 and by their UniGene Accession Number in column 7 of Tables 2 to 6, by their Swissprot Accession Number in column 8 or by the brief description of the gene or gene product in column 9 for identifying cosmetic or pharmaceutical active principles against diseases or impartment of the hair and its growth.

The present invention also relates to a process for the production of a cosmetic or pharmaceutical preparation against diseases or impairment of the hair and its growth, characterized in that

effective active principles are determined by the screening process according to the invention or by its use for identifying cosmetic or pharmaceutical active principles against diseases or impairment of the hair and its growth and

active principles found to be effective are mixed with cosmetically and pharmacologically suitable and compatible carriers.

### Tables:

TABLE 1

| Anagen | Catagen | Quotient | Significance | UniGene | Swissprot | Tag ID |
|---|---|---|---|---|---|---|
| 7.98 | 5.01 | 1.59 | 0.37 | Hs.2062 | P11473 | Vitamin D receptor |
| 1.00 | 2.00 | −2.00 | 0.60 | Hs.87409 | P07996 | Thrombospondin 1 |
| 1.00 | 3.01 | −3.01 | 0.43 | Hs.26690 | P34130 | Neurotrophin 5 |
| | | | | | | (neurotrophin 4) |

TABLE 2

| | | | | | | | |
|---|---|---|---|---|---|---|---|
| | Ana- | Kata- | | Signi- | | Swiss- | |
| Tags | gen | gen | Quotient | ficance | UniGene | prot | Tag_ID |

| | | | | | | | | |
|---|---|---|---|---|---|---|---|---|
| 1 | GCGATGGCCGT | 1.00 | 10.02 | −10.02 | 3.02 | Hs.12106 | Q96EY8 | methylmalonic aciduria |
| | | | | | | | | (cobalamin deficiency) |
| | | | | | | | | type B |
| 2 | AACTCTTGAAG | 1.00 | 10.02 | −10.02 | 2.21 | Hs.58189 | O15372 | eukaryotic translation |
| | | | | | | | | initiation factor 3, |
| | | | | | | | | subunit 3 gamma, |
| | | | | | | | | 40 kDa |
| 3 | TGTCTGCCTGA | 1.00 | 9.02 | −9.02 | 2.72 | Hs.237617 | Q8N2J7 | dipeptidylpeptidase 9 |
| | GGGGAACCCC | | | | | | | |
| | GGGAACCCCG | | | | | | | |
| 4 | CAACATTCCTG | 1.00 | 7.01 | −7.01 | 2.11 | Hs.180015 | P30046 | D-dopachrome |
| | | | | | | | | tautomerase |
| 5 | TCAATATTCTT | 1.00 | 7.01 | −7.01 | 2.11 | Hs.432458 | Q92954 | proteoglycan 4, |
| | | | | | | | | (megakaryocyte |
| | | | | | | | | stimulating factor, |
| | | | | | | | | articular superficial |
| | | | | | | | | zone protein, |
| | | | | | | | | camptodactyly, |
| | | | | | | | | arthropathy, coxa vara, |
| | | | | | | | | pericarditis syndrome) |
| 6 | GTGAGTTGGG | 1.00 | 7.01 | −7.01 | 2.11 | Hs.77897 | Q12874 | splicing factor 3a, |
| | CTGGCAGATTG | | | | | | | subunit 3, 60 kDa |
| 7 | TTCTAACTCCT | 1.00 | 7.01 | −7.01 | 2.11 | Hs.331803 | none | ESTs, Highly similar to |
| | TACCAGTGTAC | | | | | | | A32800 chaperonin |
| | | | | | | | | GroEL precursor - |
| | | | | | | | | human |
| 8 | TGAATGAGCAC | 1.00 | 7.01 | −7.01 | 2.11 | Hs.433517 | none | Homo sapiens cDNA |
| | TCTCTACAGAA | | | | | | | FLJ38383 fis, clone |
| | | | | | | | | FEBRA2003726. |
| 9 | TTGCTAGAGGG | 2.99 | 17.04 | −5.70 | 2.84 | Hs.172791 | Q9UBK9 | ubiquitously-expressed |
| | | | | | | | | transcipt |
| 10 | GCATAGTTCTA | 6.99 | 1.00 | 6.99 | 2.10 | Hs.239727 | Q02487 | (Manual) DSC2 |
| | AGAGTCATACA | | | | | | | Desmocollin-2A/2B |
| 11 | CTCCCTCTGCC | 8.98 | 1.00 | 8.98 | 2.70 | Hs.25348 | P19065 | vesicle-associated |
| | CCCCCAATTCT | | | | | | | membrane protein 2 |
| | AAAACTGGGGA | | | | | | | (synaptobrevin 2) |
| 12 | ACCGGCGCCCG | 9.98 | 1.00 | 9.98 | 2.19 | Hs.65424 | P05452 | tetranectin |
| | | | | | | | | (plasminogen binding |
| | | | | | | | | protein) |

TABLE 3

| | | | | | | | |
|---|---|---|---|---|---|---|---|
| | Ana- | Kata- | | Signifi- | | Swiss- | |
| Tags | gen | gen | Quotient | cance | UniGene | prot | Tag_ID |

| | | | | | | | | |
|---|---|---|---|---|---|---|---|---|
| 13 | ATCAGTGGCTT | 2.99 | 14.03 | −4.69 | 2.13 | Hs.89545 | P28070 | proteasome (prosome, |
| | AAGGAATCGGG | | | | | | | macropain) subunit, |
| | | | | | | | | beta type, 4 |
| 14 | ACTTTTTCAAA | 10.98 | 41.09 | −3.74 | 4.68 | manual | none | Mitochondrial major tag, |
| | | | | | | | | pos: 7503 |
| 15 | GGGTAGGGGGG | 13.97 | 43.09 | −3.08 | 4.03 | Hs.75678 | P53539 | FBJ murine |
| | CTGTACTTGTG | | | | | | | osteosarcoma viral |
| | CAGCACGGATG | | | | | | | oncogene homolog B |
| | AGATTCCAGCC | | | | | | | |
| | AAAAACATTCC | | | | | | | |
| 16 | TACCCTAAAAC | 7.98 | 23.05 | −2.89 | 2.17 | Hs.194019 | O75882 | attractin |
| 17 | ATTTGAGAAGC | 23.95 | 51.11 | −2.13 | 2.78 | manual | none | Mitochondrial major tag, |
| | | | | | | | | pos: 7313 |
| 18 | TGGAAGCAGAT | 38.92 | 19.04 | 2.04 | 2.04 | Hs.1584 | P49747 | cartilage oligomeric |
| | CGGGGTGGCCG | | | | | | | matrix protein |
| | | | | | | | | (pseudoachondroplasia, |
| | | | | | | | | epiphyseal dysplasia 1, |
| | | | | | | | | multiple) |
| 19 | AAAGCACAAGT | 40.91 | 19.04 | 2.15 | 2.33 | Hs.367762 | P02538 | keratin 6A |
| 20 | GCCGGGGTGTT | 59.88 | 25.05 | 2.39 | 3.85 | Hs.14376 | P02571 | actin, gamma 1 |
| | CTAGCCTCACG | | | | | | | |
| | CTAGCCCTCAC | | | | | | | |
| 21 | TAGGGCAATCT | 28.94 | 12.03 | 2.41 | 2.08 | Hs.380973 | P55855 | SMT3 suppressor of mif |
| | TAACAGCTACG | | | | | | | two 3 homolog 2 (yeast) |
| | CTCATTCAGCT | | | | | | | |
| | CCACTAATGGA | | | | | | | |
| 22 | TCACCGGTCAG | 36.92 | 15.03 | 2.46 | 2.64 | Hs.290070 | P06396 | gelsolin (amyloidosis, |
| | | | | | | | | Finnish type) |
| 23 | GTAATCCTGCT | 23.95 | 8.02 | 2.99 | 2.33 | manual | none | rRNA major tag |
| 24 | GTTCCCTGGCC | 24.95 | 8.02 | 3.11 | 2.52 | Hs.177415 | P35544 | (Manual) FAU, ub-like |
| | | | | | | | | protein, expressed as |
| | | | | | | | | fusion protein with |
| | | | | | | | | ribosomal protein S30 |
| 25 | GATGCCGGCAC | 16.96 | 4.01 | 4.23 | 2.35 | Hs.146559 | O43827 | angiopoietin-like factor |
| 26 | CCAGAGGCTGT | 16.96 | 4.01 | 4.23 | 2.35 | manual | none | rRNA intermediate tag |
| 27 | GGTCAGTCGGT | 13.97 | 3.01 | 4.64 | 2.11 | manual | none | rRNA major tag |
| 28 | GCAACAACACA | 18.96 | 4.01 | 4.73 | 2.80 | manual | none | rRNA intermediate tag |

TABLE 4

| | | | | | | | |
|---|---|---|---|---|---|---|---|
| | Ana- | Kata- | Quotient | Signifi- | | Swiss- | |
| Tags | gen | gen | ½ | cance | UniGene | prot | Tag_ID |

| | | | | | | | | |
|---|---|---|---|---|---|---|---|---|
| 29 | CCTCAGGATAC | 36.92 | 68.14 | −1.85 | 2.64 | manual | none | Mitochondrial |
| | | | | | | | | intermediate tag, |
| | | | | | | | | pos: 14429 |
| 30 | TTTCCTCTCAA | 43.91 | 80.17 | −1.83 | 2.96 | Hs.184510 | P31947 | stratifin |
| 31 | TCAAGCCATCA | 61.87 | 107.22 | −1.73 | 3.33 | Hs.326035 | P18146 | early growth |
| | GGATATGTGGT | | | | | | | response 1 |
| | GATTTCGTTTT | | | | | | | |
| | CTCACCTCTAG | | | | | | | |
| | CAGTTCATTAT | | | | | | | |
| 32 | TAGACCCCTTG | 63.87 | 103.21 | −1.62 | 2.64 | Hs.169476 | P04406 | glyceraldehyde- |
| | TACCATCAATA | | | | | | | 3-phosphate |
| | GCCTCCAAGGA | | | | | | | dehydrogenase |
| 33 | TTCATACACCT | 88.82 | 136.28 | −1.53 | 2.81 | manual | none | Mitochondrial |
| | | | | | | | | major tag, |
| | | | | | | | | pos: 12067 |
| 34 | TGATTTCACTT | 101.7 | 153.32 | −1.51 | 2.91 | manual | none | Mitochondrial |
| | | 9 | | | | | | major tag, |
| | | | | | | | | pos: 9302 |
| 35 | CACTACTCACC | 99.79 | 145.30 | −1.46 | 2.44 | manual | none | Mitochondrial |
| | | | | | | | | major tag, |
| | | | | | | | | pos: 14902 |
| 36 | TAAGGAGCTGA | 157.6 | 222.46 | −1.41 | 3.06 | Hs.299465 | P02383 | ribosomal |
| | | 7 | | | | | | protein S26 |
| 37 | TTGGCAGCCCA | 218.5 | 282.59 | −1.29 | 2.38 | Hs.76064 | P46776 | ribosomal |
| | GGGTCCTCTCC | 5 | | | | | | protein L27a |
| | GGGGGAGTTTC | | | | | | | |
| | GAGGGAGTTTC | | | | | | | |
| | GAGGGAATTTC | | | | | | | |
| | ATGAATTAAAA | | | | | | | |
| 38 | TCAGATTTTTG | 203.5 | 259.54 | −1.27 | 2.03 | Hs.446628 | P12750 | ribosomal |
| | TCAGATCTTTG | 8 | | | | | | protein S4, X- |
| | GTTTGTTGCCC | | | | | | | linked |
| | ATGCCCGCACC | | | | | | | |
| 39 | GTCCGAGTGCA | 81.83 | 47.10 | 1.74 | 2.66 | Hs.351316 | P30408 | transmembrane |
| | GGGACGAGTGA | | | | | | | 4 superfamily |
| | CACATATATAC | | | | | | | member 1 |
| | ATCCCTAGTAC | | | | | | | |
| 40 | GCTGGAGTTGC | 85.82 | 49.10 | 1.75 | 2.81 | Hs.41696 | Q15323 | keratin, hair, |
| | | | | | | | | acidic, 1 |
| 41 | TCGAAGCCCCC | 48.90 | 26.05 | 1.88 | 2.08 | manual | none | Mitochondrial |
| | | | | | | | | intermediate tag, |
| | | | | | | | | pos: 11417 |
| 42 | TGAGAGGGTGT | 56.88 | 29.06 | 1.96 | 2.58 | Hs.74405 | P27348 | tyrosine 3-mono- |
| | TGAAAGGGTGT | | | | | | | oxygenase/ |
| | GGCCATCTCTT | | | | | | | tryptophan 5- |
| | GAAAAGTACTA | | | | | | | monooxygenase |
| | CTCTTAATGTA | | | | | | | activation |
| | | | | | | | | protein, theta |
| | | | | | | | | polypeptide |

TABLE 5

| | | | | | | | |
|---|---|---|---|---|---|---|---|
| | Ana- | Kata- | | | | Swiss- | |
| Tags | gen | gen | Quotient | Sign. | UniGene | prot | Tag_ID |

| | | | | | | | | |
|---|---|---|---|---|---|---|---|---|
| 43 | TGGGCCCGTGT | 1.00 | 8.02 | −8.02 | 1.68 | Hs.11607 | Q8NDR0 | hypothetical |
| | ATAAAAAGCAG | | | | | | | protein FLJ32205 |
| 44 | ACTCAGAAGAG | 1.00 | 7.01 | −7.01 | 1.41 | Hs.198272 | O95178 | NADH |
| | | | | | | | | dehydrogenase |
| | | | | | | | | (ubiquinone) 1 |
| | | | | | | | | beta subcomplex, |
| | | | | | | | | 2,8 kDa |
| 45 | AGGGAGGGGCC | 1.00 | 7.01 | −7.01 | 1.41 | Hs.386793 | P22352 | glutathione |
| | | | | | | | | peroxidase 3 |
| | | | | | | | | (plasma) |
| 46 | CTTTTCTTCTG | 1.00 | 7.01 | −7.01 | 1.41 | Hs.296014 | P30876 | polymerase (RNA) |
| | | | | | | | | II (DNA directed) |
| | | | | | | | | polypeptide B, |
| | | | | | | | | 140 kDa |
| 47 | CCTGTAAAGCC | 1.00 | 7.01 | −7.01 | 1.41 | Hs.9691 | Q14344 | guanine nucleotide |
| | ACTCGTATTAG | | | | | | | binding protein (G |
| | | | | | | | | protein), alpha 13 |
| 48 | AAGGCGTTTCC | 1.00 | 7.01 | −7.01 | 1.41 | Hs.13255 | Q9Y2E2 | KIAA0930 protein |
| 49 | CCTGTGTGTGT | 1.00 | 7.01 | −7.01 | 1.41 | Hs.5894 | Q8NBF3 | hypothetical |
| | | | | | | | | protein FLJ10305 |
| 50 | CCCAGGAGCAG | 1.00 | 7.01 | −7.01 | 1.41 | Hs.22051 | Q8TBS2 | hypothetical |
| | CAGCAGGAGCA | | | | | | | protein MGC15548 |
| 51 | ACCTGCCCCTC | 1.00 | 6.01 | −6.01 | 1.81 | Hs.125262 | Q9NRG9 | achalasia, |
| | | | | | | | | adrenocortical |
| | | | | | | | | insufficiency, |
| | | | | | | | | alacrimia (Allgrove, |
| | | | | | | | | triple-A) |
| 52 | GTGGGGGGAGG | 1.00 | 6.01 | −6.01 | 1.81 | Hs.438541 | none | HLA class II region |
| | | | | | | | | expressed gene |
| | | | | | | | | KE2 |
| 53 | TCTGTGACTTC | 1.00 | 6.01 | −6.01 | 1.81 | Hs.236494 | O88386 | RAB10, member |
| | AGTTTTATTTG | | | | | | | RAS oncogene |
| | | | | | | | | family |
| 54 | GCCTGGTGACC | 1.00 | 6.01 | −6.01 | 1.81 | Hs.336916 | Q9UER7 | death-associated |
| | AGAAGAATGGG | | | | | | | protein 6 |
| 55 | TGCAAGAAGTA | 1.00 | 6.01 | −6.01 | 1.81 | Hs.206501 | O95332 | hypothetical |
| | CTTTAGCTACC | | | | | | | protein from clone |
| | CTTACGTGATT | | | | | | | 643 |
| 56 | GTTATATGCCC | 1.00 | 6.01 | −6.01 | 1.81 | Hs.13350 | none | Homo sapiens |
| | GGTTTTAGTTC | | | | | | | mRNA; cDNA |
| | | | | | | | | DKFZp586D0918 |
| | | | | | | | | (from clone |
| | | | | | | | | DKFZp586D0918) |
| 57 | TTACAACATTG | 1.00 | 6.01 | −6.01 | 1.81 | Hs.12314 | none | Homo sapiens |
| | | | | | | | | mRNA; cDNA |
| | | | | | | | | DKFZp586C1019 |
| | | | | | | | | (from clone |
| | | | | | | | | DKFZp586C1019) |
| 58 | TTTTAAACTTG | 1.00 | 6.01 | −6.01 | 1.81 | Hs.226770 | Q8TBV3 | DKFZP566C0424 |
| | TCTCCATCACT | | | | | | | protein |
| | GCTTGAACTCT | | | | | | | |
| 59 | GCTGTATGTAC | 1.00 | 6.01 | −6.01 | 1.81 | Hs.94761 | Q8TEG6 | KIAA1691 protein |
| | GCAAGGTTGGT | | | | | | | |
| 60 | TGGACAGGCAG | 2.00 | 10.02 | −5.01 | 1.66 | Hs.183800 | P46060 | Ran GTPase |
| | CTTTCCCCTTT | | | | | | | activating protein 1 |
| 61 | ACATCATACTG | 1.00 | 5.01 | −5.01 | 1.51 | Hs.61790 | Q8NCG8 | Importin 4 |
| 62 | ATGCAAGAGAG | 1.00 | 5.01 | −5.01 | 1.51 | Hs.78521 | Q8WTS6 | SET domain- |
| | | | | | | | | containing protein |
| | | | | | | | | 7 |
| 63 | CCAAGAAAGAA | 1.00 | 5.01 | −5.01 | 1.51 | Hs.169900 | Q13310 | poly(A) binding |
| | | | | | | | | protein, |
| | | | | | | | | cytoplasmic 4 |
| | | | | | | | | (inducible form) |
| 64 | GATTTGTGTTC | 1.00 | 5.01 | −5.01 | 1.51 | Hs.173125 | P30405 | peptidylprolyl |
| | | | | | | | | isomerase F |
| | | | | | | | | (cyclophilin F) |
| 65 | GCGAGAATCCA | 1.00 | 5.01 | −5.01 | 1.51 | Hs.240457 | Q96C41 | RAD9 homolog (S. |
| | | | | | | | | Pombe) |
| 66 | GGCCAGCAAGT | 1.00 | 5.01 | −5.01 | 1.51 | Hs.271353 | Q15830 | mutY homolog (E. |
| | | | | | | | | coli) |
| 67 | GGTGACAGAGA | 1.00 | 5.01 | −5.01 | 1.51 | Hs.267632 | P82094 | TATA element |
| | | | | | | | | modulatory factor 1 |
| 68 | TGTAAAGATTT | 1.00 | 5.01 | −5.01 | 1.51 | Hs.4859 | Q8NI48 | cyclin L ania-6a |
| 69 | TGTATACAAGG | 1.00 | 5.01 | −5.01 | 1.51 | Hs.349650 | P04049 | v-raf-1 murine |
| | | | | | | | | leukemia viral |
| | | | | | | | | oncogene homolog |
| | | | | | | | | 1 |
| 70 | TTGCCTTTTTA | 1.00 | 5.01 | −5.01 | 1.51 | Hs.4311 | O95605 | SUMO-1 activating |
| | | | | | | | | enzyme subunit 2 |
| 71 | TTGTGGGATCT | 1.00 | 5.01 | −5.01 | 1.51 | Hs.278540 | P06705 | protein |
| | | | | | | | | phosphatase 3 |
| | | | | | | | | (formerly 2B), |
| | | | | | | | | regulatory subunit |
| | | | | | | | | B, 19 kDa, alpha |
| | | | | | | | | isoform (calci- |
| | | | | | | | | neurin B, type I) |
| 72 | AGCCCTGGAGT | 1.00 | 5.01 | −5.01 | 1.51 | Hs.20047 | Q8WYX7 | zinc finger protein, |
| | ACCGCCGGGCT | | | | | | | subfamily 2A |
| | | | | | | | | (FYVE domain |
| | | | | | | | | containing), 1 |
| 73 | TTGCCGGTTAA | 1.00 | 5.01 | −5.01 | 1.51 | Hs.405813 | Q92530 | proteasome |
| | ACTGGAAGGAG | | | | | | | (prosome, |
| | | | | | | | | macropain) |
| | | | | | | | | inhibitor subunit 1 |
| | | | | | | | | (P131) |
| 74 | CAGAGTTGTAT | 1.00 | 5.01 | −5.01 | 1.51 | Hs.5672 | Q8NHE5 | golgi membrane |
| | AAATGCGAACA | | | | | | | protein SB140 |
| 75 | GCTCTGCCCTC | 1.00 | 5.01 | −5.01 | 1.51 | Hs.68257 | P35269 | general |
| | GCTCTGCCCCC | | | | | | | transcription factor |
| | | | | | | | | IIF, polypeptide 1, |
| | | | | | | | | 74 kDa |
| 76 | TCTTTGTCTAA | 1.00 | 5.01 | −5.01 | 1.51 | Hs.6838 | P52199 | ras homolog gene |
| | GGATATATCCA | | | | | | | family, member E |
| | ATAGTGCTTCG | | | | | | | |
| 77 | AGCCTACAGGT | 1.00 | 5.01 | −5.01 | 1.51 | Hs.278359 | Q8N1P7 | Homo sapiens |
| | | | | | | | | cDNA FLJ38020 |
| | | | | | | | | fis, clone |
| | | | | | | | | CTONG2012843, |
| | | | | | | | | weakly similar to |
| | | | | | | | | Human non-lens |
| | | | | | | | | beta gamma- |
| | | | | | | | | crystalline like |
| | | | | | | | | protein (AIM1) |
| | | | | | | | | mRNA. |
| 78 | ATCCACCCGCC | 1.00 | 5.01 | −5.01 | 1.51 | Hs.251337 | none | ESTs, Weakly |
| | | | | | | | | similar to |
| | | | | | | | | hypothetical |
| | | | | | | | | protein FLJ20489 |
| 79 | CCAGAACTCTT | 1.00 | 5.01 | −5.01 | 1.51 | Hs.184183 | Q9H5Z4 | Homo sapiens |
| | | | | | | | | cDNA: FLJ22755 |
| | | | | | | | | fis, clone |
| | | | | | | | | KAIA0769. |
| 80 | CCCTGAAGAGC | 1.00 | 5.01 | −5.01 | 1.51 | Hs.34579 | Q8WY60 | hypothetical |
| | | | | | | | | protein FLJ10948 |
| 81 | CGCCCGTCGTG | 1.00 | 5.01 | −5.01 | 1.51 | Hs.134742 | Q9NPT2 | hypothetical |
| | | | | | | | | protein |
| | | | | | | | | DKFZp547D065 |
| 82 | CTGGGATCATC | 1.00 | 5.01 | −5.01 | 1.51 | Hs.336425 | Q96GX2 | Homo sapiens, |
| | | | | | | | | clone MGC: 17296 |
| | | | | | | | | IMAGE: 3460701, |
| | | | | | | | | mRNA, complete |
| | | | | | | | | cds |
| 83 | GCCACAGCCAG | 1.00 | 5.01 | −5.01 | 1.51 | Hs.198037 | O60339 | KIAA0599 protein |
| 84 | TGCCGTGCCTG | 1.00 | 5.01 | −5.01 | 1.51 | Hs.347187 | Q96FD1 | Homo sapiens |
| | | | | | | | | cDNA: FLJ21092 |
| | | | | | | | | fis, clone |
| | | | | | | | | CAS03646. |
| 85 | TGTCGGGAAAT | 1.00 | 5.01 | −5.01 | 1.51 | Hs.301065 | O75033 | KIAA0445 gene |
| | | | | | | | | product |
| 86 | CCACAACCTGG | 5.99 | 1.00 | 5.99 | 1.80 | Hs.101742 | Q96E34 | ribosomal large |
| | | | | | | | | subunit |
| | | | | | | | | pseudouridine |
| | | | | | | | | synthase C like |
| 87 | GCCGCCGAGCC | 5.99 | 1.00 | 5.99 | 1.80 | Hs.115232 | Q15428 | splicing factor 3a, |
| | CCCCCAATGTT | | | | | | | subunit 2, 66 kDa |
| | CCCCCAATGCT | | | | | | | |
| 88 | GCTTACCTTTC | 5.99 | 1.00 | 5.99 | 1.80 | Hs.7753 | O43852 | calumenin |
| | CACTTGAAAAG | | | | | | | |
| 89 | TGTTAGCCTGT | 5.99 | 1.00 | 5.99 | 1.80 | Hs.92384 | O75915 | vitamin A |
| | TATAGGCCGAA | | | | | | | responsive; |
| | GTCTAGAATCT | | | | | | | cytoskeleton |
| | CTGCCATAGAT | | | | | | | related |
| 90 | CCTGTACCCCA | 6.99 | 1.00 | 6.99 | 1.40 | Hs.32317 | Q8NBD5 | high-mobility group |
| | | | | | | | | 20B |
| 91 | CGGAGTCCATT | 6.99 | 1.00 | 6.99 | 1.40 | Hs.155595 | Q15019 | neural precursor |
| | | | | | | | | cell expressed, |
| | | | | | | | | developmentally |
| | | | | | | | | down-regulated 5 |
| 92 | GAGCAGCGCCC | 6.99 | 1.00 | 6.99 | 1.40 | Hs.112408 | P31151 | S100 calcium |
| | | | | | | | | binding protein A7 |
| | | | | | | | | (psoriasin 1) |
| 93 | GTAGCAGGGCT | 6.99 | 1.00 | 6.99 | 1.40 | Hs.302441 | Q9H269 | vacuolar protein |
| | | | | | | | | sorting 16 (yeast) |
| 94 | TGAGGGGTGAA | 6.99 | 1.00 | 6.99 | 1.40 | Hs.268530 | Q13098 | G protein pathway |
| | | | | | | | | suppressor 1 |
| 95 | AAGTCATTCAG | 6.99 | 1.00 | 6.99 | 1.40 | Hs.274416 | P56556 | NADH |
| | AGGCTGGACGA | | | | | | | dehydrogenase |
| | | | | | | | | (ubiquinone) 1 |
| | | | | | | | | alpha subcomplex, |
| | | | | | | | | 6, 14 kDa |
| 96 | GTGTGAGTGTG | 6.99 | 1.00 | 6.99 | 1.40 | Hs.7838 | Q9UHC7 | makorin, ring finger |
| | ATGAGCTGGAA | | | | | | | protein, 1 |
| 97 | CGCATTAAAGC | 6.99 | 1.00 | 6.99 | 1.40 | Hs.432368 | Q8N9S5 | Homo sapiens |
| | | | | | | | | cDNA FLJ30256 |
| | | | | | | | | fis, clone |
| | | | | | | | | BRACE2002458. |
| 98 | CTCGGCCAGAG | 6.99 | 1.00 | 6.99 | 1.40 | Hs.311611 | none | EST |
| 99 | CAAGCAGGACA | 7.98 | 1.00 | 7.98 | 1.66 | Hs.424551 | Q9Y3Q3 | integral type I |
| | | | | | | | | protein |
| 100 | TGATGTCTGGT | 7.98 | 1.00 | 7.98 | 1.66 | Hs.83883 | Q969W9 | transmembrane, |
| | | | | | | | | prostate androgen |
| | | | | | | | | induced RNA |
| 101 | TTCTTATTTTA | 7.98 | 1.00 | 7.98 | 1.66 | Hs.406423 | Q13435 | splicing factor 3b, |
| | GTGGCTGAGCA | | | | | | | subunit 2, 145 kDa |
| 102 | CAGGAGAACTG | 7.98 | 1.00 | 7.98 | 1.66 | Hs.150614 | Q8NAL3 | hypothetical |
| | AGTGAGGATAG | | | | | | | protein FLJ35155 |
| 103 | CAGCTTGCAAA | 8.98 | 1.00 | 8.98 | 1.92 | Hs.105465 | Q15356 | small nuclear |
| | | | | | | | | ribonucleoprotein |
| | | | | | | | | polypeptide F |
| 104 | GTTTATGGATA | 8.98 | 1.00 | 8.98 | 1.92 | Hs.365706 | P08493 | matrix Gla protein |
| | | 1.00 | 8.02 | −8.02 | 1.68 | Hs.284162 | Q8N6S8 | chromosome 15 |
| | | | | | | | | open reading |
| | | | | | | | | frame 15 |
| | | 1.00 | 8.02 | −8.02 | 1.68 | Hs.71746 | Q8NDH3 | aminopeptidase- |
| | | | | | | | | like 1 |
| | | 1.00 | 7.01 | −7.01 | 1.41 | Hs.183037 | P10644 | protein kinase, |
| | | | | | | | | cAMP-dependent, |
| | | | | | | | | regulatory, type I, |
| | | | | | | | | alpha (tissue |
| | | | | | | | | specific |
| | | | | | | | | extinguisher 1) |
| | | 1.00 | 5.01 | −5.01 | 1.51 | Hs.79530 | Q9NPL8 | chromosome 3 |
| | | | | | | | | open reading |
| | | | | | | | | frame 1 |
| | | 1.00 | 7.01 | −7.01 | 1.41 | Hs.323463 | Q8N4E8 | hypothetical |
| | | | | | | | | protein MGC8902 |

TABLE 6

| | | | | | | | |
|---|---|---|---|---|---|---|---|
| | Ana- | Kata- | | Signifi- | | Swiss- | |
| Tags | gen | gen | Quotient | cance | UniGene | prot | Tag_ID |

| | | | | | | | | |
|---|---|---|---|---|---|---|---|---|
| 105 | GTTTGCAAGTG | 2.00 | 9.02 | −4.51 | 1.42 | Hs.151787 | Q15029 | U5 snRNP- |
| | | | | | | | | specific0 |
| | | | | | | | | protein, 116 |
| | | | | | | | | kD |
| 106 | TTACTAAATGG | 2.99 | 11.02 | −3.69 | 1.46 | Hs.155560 | P27824 | calnexin |
| | TAACAGTTGTG | | | | | | | |
| | CGGGATGCAGA | | | | | | | |
| | CCTCACTTTTT | | | | | | | |
| | CCTCACTTTCT | | | | | | | |
| | ACATATACTGT | | | | | | | |
| | AAGCAAACTAA | | | | | | | |
| 107 | TACAAAACCAT | 3.99 | 12.03 | −3.02 | 1.32 | Hs.79110 | Q8NB06 | Nucleolin |
| | GTTTTTGCTTC | | | | | | | |
| | GAAGACGGTGA | | | | | | | |
| | ATAAAACATTC | | | | | | | |
| 108 | AGGCTTTATGG | 6.99 | 20.04 | −2.87 | 1.92 | Hs.24385 | none | Human |
| | | | | | | | | hbc647 |
| | | | | | | | | mRNA |
| | | | | | | | | sequence. |
| 109 | TTCAGTGAAGG | 6.99 | 18.04 | −2.58 | 1.55 | Hs.2795 | P00338 | lactate |
| | TCTTGTGTATA | | | | | | | dehydro- |
| | TCTTGTGCATA | | | | | | | genase A |
| 110 | CCTGTGCCTGG | 6.99 | 17.04 | −2.44 | 1.37 | Hs.95972 | P40967 | silver |
| | CCTGGTCAAGA | | | | | | | homolog |
| | | | | | | | | (mouse) |
| 111 | CGTTCCTGCGG | 7.98 | 19.04 | −2.39 | 1.46 | Hs.75424 | P41134 | inhibitor of |
| | | | | | | | | DNA binding |
| | | | | | | | | 1, dominant |
| | | | | | | | | negative |
| | | | | | | | | helix-loop- |
| | | | | | | | | helix protein |
| 112 | TGAGGGAATAA | 14.97 | 32.07 | −2.14 | 1.89 | Hs.83848 | P00938 | triosephos- |
| | | | | | | | | phate |
| | | | | | | | | isomerase 1 |
| 113 | TTGAATGAACA | 9.98 | 21.04 | −2.11 | 1.31 | Hs.372673 | O14979 | heterogen- |
| | TTAAACCTCAA | | | | | | | eous nuclear |
| | GATACAAAAAC | | | | | | | ribonucleo- |
| | CAACTTTAGGG | | | | | | | protein D-like |
| | AAATGATACAA | | | | | | | |
| | AAAGTGGACCT | | | | | | | |
| 114 | GTGCCCTGTTG | 11.98 | 24.05 | −2.01 | 1.34 | Hs.278411 | Q9Y2A7 | NCK- |
| | | | | | | | | associated |
| | | | | | | | | protein 1 |
| 115 | CACGCAATGCT | 13.97 | 5.01 | 2.79 | 1.37 | Hs.375592 | Q08117 | amino- |
| | | | | | | | | terminal |
| | | | | | | | | enhancer of |
| | | | | | | | | split |
| 116 | TATGCCCGAAT | 19.96 | 7.01 | 2.85 | 1.89 | Hs.41690 | Q14574 | desmocollin |
| | CAGGAGTGTGC | | | | | | | 3 |
| 117 | TGACCCCACAG | 11.98 | 4.01 | 2.99 | 1.30 | Hs.356578 | none | mitochondrial |
| | | | | | | | | ribosomal |
| | | | | | | | | protein L54 |
| 118 | TTTGGGGCTGG | 11.98 | 4.01 | 2.99 | 1.30 | Hs.7476 | Q99437 | ATPase, H+ |
| | | | | | | | | transporting, |
| | | | | | | | | lysosomal |
| | | | | | | | | 21 kDa, V0 |
| | | | | | | | | subunit c″ |
| 119 | GCGGGAGGGCT | 14.97 | 5.01 | 2.99 | 1.56 | Hs.399736 | P36404 | ADP- |
| | | | | | | | | ribosylation |
| | | | | | | | | factor-like 2 |
| 120 | TGTGGGTGCTG | 14.97 | 5.01 | 2.99 | 1.56 | Hs.194657 | P12830 | cadherin 1, |
| | | | | | | | | type 1 E- |
| | | | | | | | | cadherin |
| | | | | | | | | (epithelial) |
| 121 | CTGTGACACAG | 12.97 | 4.01 | 3.23 | 1.50 | Hs.432970 | P78371 | chaperonin |
| | | | | | | | | containing |
| | | | | | | | | TCP1, |
| | | | | | | | | subunit 2 |
| | | | | | | | | (beta) |
| 122 | GGCTTTGGAGT | 10.98 | 3.01 | 3.65 | 1.45 | Hs.90918 | Q9Y2Q7 | chromosome |
| | | | | | | | | 11 open |
| | | | | | | | | reading |
| | | | | | | | | frame 10 |
| 123 | AGAATCGCTTG | 8.98 | 2.00 | 4.49 | 1.41 | manual | none | Alu-repeat |
| 124 | CCCTGGGTTCT | 8.98 | 2.00 | 4.49 | 1.41 | Hs.430150 | P02792 | ferritin, light |
| | | | | | | | | polypeptide |
| 125 | GTGAAACCTCG | 8.98 | 2.00 | 4.49 | 1.41 | Hs.274417 | Q9Y676 | mitochondrial |
| | | | | | | | | ribosomal |
| | | | | | | | | protein S18B |
| 126 | TTACGAGGAAG | 8.98 | 2.00 | 4.49 | 1.41 | Hs.300471 | P55735 | SEC13-like 1 |
| | | | | | | | | (S. cere- |
| | | | | | | | | visiae) |
| 127 | CAGCGCCTGGC | 4.99 | 1.00 | 4.99 | 1.50 | Hs.110571 | O75293 | growth arrest |
| | AACTCCCAGTT | | | | | | | and DNA- |
| | | | | | | | | damage- |
| | | | | | | | | inducible, |
| | | | | | | | | beta |
| 128 | AGGTGCAGAGG | 4.99 | 1.00 | 4.99 | 1.50 | Hs.13501 | O00541 | pescadillo |
| | | | | | | | | homolog 1, |
| | | | | | | | | containing |
| | | | | | | | | BRCT |
| | | | | | | | | domain |
| | | | | | | | | (zebrafish) |
| 129 | ATGTACTAAAG | 4.99 | 1.00 | 4.99 | 1.50 | Hs.250897 | Q92734 | TRK-fused |
| | | | | | | | | gene |
| 130 | GACGCAGAAGT | 4.99 | 1.00 | 4.99 | 1.50 | Hs.296426 | O95782 | adaptor- |
| | | | | | | | | related |
| | | | | | | | | protein |
| | | | | | | | | complex 2, |
| | | | | | | | | alpha 1 |
| | | | | | | | | subunit |
| 131 | GAGCAGCTGGA | 4.99 | 1.00 | 4.99 | 1.50 | Hs.166887 | Q99829 | copine I |
| 132 | GGGATCGCCCC | 4.99 | 1.00 | 4.99 | 1.50 | Hs.284261 | Q9U106 | NSFL1 (p97) |
| | | | | | | | | cofactor |
| | | | | | | | | (p47) |
| 133 | GTTTCTTCCCT | 4.99 | 1.00 | 4.99 | 1.50 | Hs.290874 | Q8N672 | selenoprotein |
| | | | | | | | | H |
| 134 | GCTAAGTATTT | 4.99 | 1.00 | 4.99 | 1.50 | Hs.380963 | Q9UIV1 | CCR4-NOT |
| | GCCCATTTTAT | | | | | | | transciption |
| | CTTGTATATAG | | | | | | | complex, |
| | ATATTACAGTG | | | | | | | subunit 7 |
| 135 | CAAAGGCTGTG | 4.99 | 1.00 | 4.99 | 1.50 | Hs.75412 | P55145 | arginine-rich, |
| | AGGGGATTCCC | | | | | | | mutated in |
| | | | | | | | | early stage |
| | | | | | | | | tumors |
| 136 | TAAATGATCAG | 2.00 | 9.02 | −4.51 | 1.42 | Hs.190452 | O15071 | KIAA0365 |
| | GTGTAACCCCG | | | | | | | gene product |
| | GTGCGTGCTGC | | | | | | | |
| | GCCTGGGCTCC | | | | | | | |
| | CCAGGCCCTGG | | | | | | | |
| 137 | TTGTCGATGGG | 8.98 | 2.00 | 4.49 | 1.41 | Hs.55505 | Q9BVJ7 | hypothetical |
| | | | | | | | | protein |
| | | | | | | | | FLJ20442 |
| 138 | GTGGCGCACAC | 4.99 | 1.00 | 4.99 | 1.50 | Hs.375756 | none | Homo |
| | | | | | | | | sapiens, |
| | | | | | | | | clone |
| | | | | | | | | IMAGE: 4153 |
| | | | | | | | | 384, mRNA |
| 139 | TCAGCCGCTAC | 4.99 | 1.00 | 4.99 | 1.50 | Hs.39132 | Q96LW7 | hypothetical |
| | | | | | | | | protein |
| | | | | | | | | MGC11115 |
| 140 | ACCCGCCGGGC | 25.95 | 11.02 | 2.35 | 1.84 | manual | none | rRNA major |
| | | | | | | | | tag |
| 141 | TACTGCTCGGA | 10.98 | 3.01 | 3.65 | 1.45 | manual | none | Mitochondrial |
| | | | | | | | | antisense |
| | | | | | | | | tag, pos:- |
| | | | | | | | | 13715 |
| | | 3.99 | 12.03 | −3.02 | 1.32 | Hs.278589 | P78347 | general |
| | | | | | | | | transcription |
| | | | | | | | | factor II, i |
| | | 5.99 | 17.04 | −2.84 | 1.66 | Hs.406404 | Q14103 | heterogene- |
| | | | | | | | | ous nuclear |
| | | | | | | | | ribonucleo- |
| | | | | | | | | protein D |
| | | | | | | | | (AU-rich |
| | | | | | | | | element RNA |
| | | | | | | | | binding |
| | | | | | | | | protein 1, |
| | | | | | | | | 37 kDa) |
| | | 6.99 | 19.04 | −2.72 | 1.73 | Hs.356531 | P07900 | heat shock |
| | | | | | | | | 90 kDa |
| | | | | | | | | protein 1, |
| | | | | | | | | alpha |
| | | 10.98 | 23.05 | −2.10 | 1.40 | Hs.334842 | P05209 | tubulin, |
| | | | | | | | | alpha, |
| | | | | | | | | ubiquitous |
| | | 8.98 | 20.04 | −2.23 | 1.38 | Hs.301885 | none | Homo |
| | | | | | | | | sapiens |
| | | | | | | | | cDNA |
| | | | | | | | | FLJ11346 fis, |
| | | | | | | | | clone |
| | | | | | | | | PLACE1010 |
| | | | | | | | | 900. |
| | | 4.99 | 1.00 | 4.99 | 1.50 | Hs.375756 | none | Homo |
| | | | | | | | | sapiens, |
| | | | | | | | | clone |
| | | | | | | | | IMAGE: 4153 |
| | | | | | | | | 384, mRNA |
| | | 8.98 | 20.04 | −2.23 | 1.38 | Hs.153 | P18124 | ribosomal |
| | | | | | | | | protein L7 |

TABLE 7

| | | | | | | | |
|---|---|---|---|---|---|---|---|
| | Ana- | Kata- | | | GO- | | |
| Tags | gen | gen | Quot. | Signf. | Number | Description | Swissprot |

| | | | | | | | | |
|---|---|---|---|---|---|---|---|---|
| | | 5 | 20 | 4 | 2.62 | GO0003678 | DNA helicase | 11 matches |
| | | | | | | | activity | |
| 142 | ACTATAGAGAC | 0 | 2 | 4 | 0.6 | GO0003678 | DEAD/H (Asp-Glu- | [Swissprot:tr\|Q924 |
| | | | | | | | Ala-Asp/His) box | 98;tr\|Q92770;tr\|Q9 |
| | | | | | | | polypeptide 11 | 2998;tr\|Q92999;tr\| |
| | | | | | | | (CHL1-like | Q93000;tr\|Q96FC9;] |
| | | | | | | | helicase homolog, | |
| | | | | | | | S. cerevisiae) | |
| 143 | CCCTGGTGGGC | 0 | 2 | 4 | 0.6 | GO0003678 | RecQ protein-like 4 | [Swissprot:sp\|O94 |
| | | | | | | | | 761;tr\|Q96DW2;tr\| |
| | | | | | | | | Q96F55;] |
| 144 | CCGCACCTCCA | 1 | 0 | −2 | 0.3 | GO0003678 | RecQ protein-like 4 | [Swissprot:sp\|O94 |
| | | | | | | | | 761;tr\|Q96DW2;tr\| |
| | | | | | | | | Q96F55;] |
| 145 | CAGGCGTGCAC | 3 | 6 | 2 | 0.47 | GO0003678 | RecQ protein-like 5 | [Swissprot:sp\|O94 |
| | | | | | | | | 762;tr\|Q8WYH5;tr\| |
| | | | | | | | | Q9BSD6;tr\|Q9BW8 |
| | | | | | | | | 0;tr\|Q9H0B1;] |
| 146 | TCAGTATTCTA | 0 | 1 | 2 | 0.3 | GO0003678 | RecQ protein-like 5 | [Swissprot:sp\|O94 |
| | | | | | | | | 762;tr\|Q8WYH5;tr\| |
| | | | | | | | | Q9BSD6;tr\|Q9BW8 |
| | | | | | | | | 0;tr\|Q9H0B1;] |
| 147 | TCGAGGACAGA | 0 | 1 | 2 | 0.3 | GO0003678 | RecQ protein-like 5 | [Swissprot:sp\|O94 |
| | | | | | | | | 762;tr\|Q8WYH5;tr\| |
| | | | | | | | | Q9BSD6;tr\|Q9BW8 |
| | | | | | | | | 0;tr\|Q9H0B1;] |
| 148 | AAGTGAGATGG | 0 | 3 | 6 | 0.91 | GO0003678 | RuvB-like 1 (E. | [Swissprot:sp\|Q9Y |
| | | | | | | | coli) | 265;] |
| 149 | GAATTGAAATA | 0 | 1 | 2 | 0.3 | GO0003678 | SWI/SNF related, | [Swissprot:tr\|Q96A |
| | | | | | | | matrix associated, | Y1;tr\|Q9NXQ5;tr\|Q |
| | | | | | | | actin dependent | 9NZC9;tr\|Q9UFH3; |
| | | | | | | | regulator of | tr\|Q9UI93;] |
| | | | | | | | chromatin, | |
| | | | | | | | subfamily a-like 1 | |
| 150 | GCAGAACCATT | 0 | 1 | 2 | 0.3 | GO0003678 | alpha | [Swissprot:sp\|P461 |
| | | | | | | | thalassemia/mental | 00;] |
| | | | | | | | retardation | |
| | | | | | | | syndrome X-linked | |
| | | | | | | | (RAD54 homolog, | |
| | | | | | | | S. cerevisiae) | |
| 151 | TACACCCGCTC | 1 | 2 | 2 | 0.21 | GO0003678 | excision repair | [Swissprot:sp\|P194 |
| | | | | | | | cross- | 47;] |
| | | | | | | | complementing | |
| | | | | | | | rodent repair | |
| | | | | | | | deficiency, | |
| | | | | | | | complementation | |
| | | | | | | | group 3 | |
| | | | | | | | (xeroderma | |
| | | | | | | | pigmentosum | |
| | | | | | | | group B | |
| | | | | | | | complementing) | |
| 152 | TGGCCAGATGC | 0 | 1 | 2 | 0.3 | GO0003678 | immunoglobulin | [Swissprot:sp\|P389 |
| | | | | | | | mu binding protein | 35;] |
| | | | | | | | 2 | |
| | | 12 | 2 | −6 | 2.12 | GO0003831 | beta-N-acetyl- | 4 matches |
| | | | | | | | glucosaminyl | |
| | | | | | | | glycopeptide | |
| | | | | | | | beta-1,4-galacto- | |
| | | | | | | | syltransferase | |
| | | | | | | | activity | |
| 153 | ATCCGCCACTC | 1 | 0 | −2 | 0.3 | GO0003831 | UDP- | [Swissprot:sp\|P152 |
| | | | | | | | Gal:betaGlcNAc | 91;] |
| | | | | | | | beta 1,4-galacto- | |
| | | | | | | | syltransferase, | |
| | | | | | | | polypeptide 1 | |
| 154 | TCCCAGAGACC | 2 | 0 | −4 | 0.6 | GO0003831 | UDP- | [Swissprot:sp\|P152 |
| | | | | | | | Gal:betaGlcNAc | 91;] |
| | | | | | | | beta 1,4- | |
| | | | | | | | galactosyltransfer- | |
| | | | | | | | ase, polypeptide 1 | |
| 155 | GGAGGCAGGTG | 8 | 2 | −4 | 1.18 | GO0003831 | UDP- | [Swissprot:sp\|O60 |
| | | | | | | | Gal:betaGlcNAc | 909;tr\|Q9BUP6;] |
| | | | | | | | beta 1,4- | |
| | | | | | | | galactosyltransfer- | |
| | | | | | | | ase, polypeptide 2 | |
| 156 | GAGAGAAGAGT | 1 | 0 | −2 | 0.3 | GO0003831 | UDP- | [Swissprot:sp\|O60 |
| | | | | | | | Gal:betaGlcNAc | 512;tr\|Q9BPZ4;tr\|Q |
| | | | | | | | beta 1,4- | 9H8T2;] |
| | | | | | | | galactosyltransfer- | |
| | | | | | | | ase, polypeptide 3 | |
| | | 76 | 47 | −1.62 | 2.02 | GO0003924 | GTPase activity | 42 matches |
| 157 | AGGAACACAAA | 3 | 1 | −3 | 0.42 | GO0003924 | (Manual) EIF2S3 | [Swissprot:sp\|P410 |
| | | | | | | | Eukaryotic | 91;] |
| | | | | | | | translation initiation | |
| | | | | | | | factor 2, subunit 3 | |
| | | | | | | | gamma, 52 kDa | |
| 158 | GGCCTACATCC | 0 | 1 | 2 | 0.3 | GO0003924 | ADP-ribosylation | [Swissprot:sp\|P328 |
| | | | | | | | factor 1 | 89;] |
| 159 | TGCTTGTCCCT | 8 | 4 | −2 | 0.57 | GO0003924 | ADP-ribosylation | [Swissprot:sp\|P328 |
| | | | | | | | factor 1 | 89;] |
| 160 | TGGCAAACGTG | 4 | 0 | −8 | 1.2 | GO0003924 | ADP-ribosylation | [Swissprot:sp\|P328 |
| | | | | | | | factor 1 | 89;] |
| 161 | AGGACTTTGCC | 2 | 1 | −2 | 0.2 | GO0003924 | ADP-ribosylation | [Swissprot:sp\|P165 |
| | | | | | | | factor 3 | 87;] |
| 162 | CCCAGCAAGAG | 1 | 0 | −2 | 0.3 | GO0003924 | ADP-ribosylation | [Swissprot:sp\|P165 |
| | | | | | | | factor 3 | 87;] |
| 163 | CTGTTACAGGT | 0 | 1 | 2 | 0.3 | GO0003924 | ADP-ribosylation | [Swissprot:sp\|P364 |
| | | | | | | | factor domain | 06;] |
| | | | | | | | protein 1, 64 kDa | |
| 164 | TTAATAAAATA | 1 | 0 | −2 | 0.3 | GO0003924 | G1 to S phase | [Swissprot:sp\|P151 |
| | | | | | | | transition 1 | 70;tr\|Q96GF2;] |
| 165 | TTACAAAGGCA | 0 | 1 | 2 | 0.3 | GO0003924 | G1 to S phase | [Swissprot:sp\|P151 |
| | | | | | | | transition 1 | 70;tr\|Q96GF2;] |
| 166 | TTTGAGACCTG | 1 | 0 | −2 | 0.3 | GO0003924 | G1 to S phase | [Swissprot:sp\|P151 |
| | | | | | | | transition 1 | 70;tr\|Q96GF2;] |
| 167 | GTAATGTCCAT | 0 | 1 | 2 | 0.3 | GO0003924 | KIAA0820 protein | [Swissprot:sp\|Q9U |
| | | | | | | | | Q16;] |
| 168 | GCCAACGGCGT | 1 | 0 | −2 | 0.3 | GO0003924 | MLL septin-like | [Swissprot:tr\|Q96Q |
| | | | | | | | fusion | F3;tr\|Q96QF4;tr\|Q9 |
| | | | | | | | | 6QF5;tr\|Q9HA04;tr\| |
| | | | | | | | | Q9HC74;tr\|Q9UG4 |
| | | | | | | | | 0;tr\|Q9UHD8;tr\|Q9 |
| | | | | | | | | Y5W4;] |
| 169 | TGGCCTGCCCA | 7 | 3 | −2.33 | 0.64 | GO0003924 | MLL septin-like | [Swissprot:tr\|Q96Q |
| | | | | | | | fusion | F3;tr\|Q96QF4;tr\|Q9 |
| | | | | | | | | 6QF5;tr\|Q9HA04;tr\| |
| | | | | | | | | Q9HC74;tr\|Q9UG4 |
| | | | | | | | | 0;tr\|Q9UHD8;tr\|Q9 |
| | | | | | | | | Y5W4;] |
| 170 | GACACGAACAA | 1 | 1 | 1 | 0 | GO0003924 | RAS, | [Swissprot:tr\|Q9HC |
| | | | | | | | dexamethasone- | 43;tr\|Q9Y272;] |
| | | | | | | | induced 1 | |
| 171 | CTCGGTGATGT | 7 | 3 | −2.33 | 0.64 | GO0003924 | Ras homolog | [Swissprot:sp\|Q15 |
| | | | | | | | enriched in brain 2 | 382;] |
| 172 | ATATCTTTGCT | 1 | 0 | −2 | 0.3 | GO0003924 | Ras-like without | [Swissprot:tr\|O152 |
| | | | | | | | CAAX 2 | 95;tr\|Q8TD69;tr\|Q8 |
| | | | | | | | | WVF6;tr\|Q92964;tr\| |
| | | | | | | | | Q99578;] |
| 173 | CTGAAGCTAAG | 0 | 1 | 2 | 0.3 | GO0003924 | SAM domain and | [Swissprot:sp\|Q9Y |
| | | | | | | | HD domain 1 | 3Z3;tr\|Q8N491;] |
| 174 | GCGAAACCCAG | 1 | 0 | −2 | 0.3 | GO0003924 | SAM domain and | [Swisprot:sp\|Q9Y3 |
| | | | | | | | HD domain 1 | Z3;tr\|Q8N491;] |
| 175 | GTTTGCAAGTG | 2 | 9 | 4.5 | 1.42 | GO0003924 | U5 snRNP-specific | [Swisprot:sp\|Q150 |
| | | | | | | | protein, 116 kD | 29;tr\|Q8IXJ3;] |
| 176 | GGGGTGCTGTG | 2 | 1 | −2 | 0.2 | GO0003924 | dynamin 1 | [Swisprot:sp\|Q051 |
| | | | | | | | | 93;] |
| 177 | TGGAGACTGGC | 0 | 2 | 4 | 0.6 | GO0003924 | dynamin 1-like | [Swissprot:tr\|O004 |
| | | | | | | | | 29;tr\|O14541;tr\|O6 |
| | | | | | | | | 0709;tr\|Q8TBT7;tr\| |
| | | | | | | | | Q9Y5J2;] |
| 178 | CCTCCCTGATG | 2 | 4 | 2 | 0.35 | GO0003924 | dynamin 2 | [Swissprot:sp\|P505 |
| | | | | | | | | 70;tr\|Q8N1K8;] |
| 179 | ATGTATAATTT | 1 | 0 | −2 | 0.3 | GO0003924 | eukaryotic | [Swissprot:sp\|P410 |
| | | | | | | | translation initiation | 91;] |
| | | | | | | | factor 2, subunit 3 | |
| | | | | | | | gamma, 52 kDa | |
| 180 | TTGGCTAGGCC | 0 | 1 | 2 | 0.3 | GO0003924 | eukaryotic | [Swissprot:sp\|P410 |
| | | | | | | | translation initiation | 91;] |
| | | | | | | | factor 2, subunit 3 | |
| | | | | | | | gamma, 52 kDa | |
| 181 | CTTGACACACA | 1 | 0 | −2 | 0.3 | GO0003924 | eukaryotic | [Swissprot:sp\|P550 |
| | | | | | | | translation initiation | 10;] |
| | | | | | | | factor 5 | |
| 182 | TTCAGGGCTTC | 1 | 2 | 2 | 0.21 | GO0003924 | eukaryotic | [Swissprot:sp\|P550 |
| | | | | | | | translation initiation | 10;] |
| | | | | | | | factor 5 | |
| 183 | GGCAGGAGTAG | 2 | 1 | −2 | 0.2 | GO0003924 | guanylate binding | [Swissprot:sp\|P324 |
| | | | | | | | protein 1, | 55;] |
| | | | | | | | interferon- | |
| | | | | | | | inducible, 67 kDa | |
| 184 | AATGAGCAACT | 0 | 1 | 2 | 0.3 | GO0003924 | guanylate binding | [Swissprot:sp\|P324 |
| | | | | | | | protein 2, | 56;tr\|Q8TCE5;] |
| | | | | | | | interferon-inducible | |
| 185 | GCTTAATGTGT | 1 | 0 | −2 | 0.3 | GO0003924 | mitochondrial GTP | [Swissprot:tr\|Q8TC |
| | | | | | | | binding protein | Y6;tr\|Q8WUW9;tr\| |
| | | | | | | | | Q969G4;tr\|Q969Y2; |
| | | | | | | | | tr\|Q96H44;] |
| 186 | GCAGCTATGTG | 2 | 0 | −4 | 0.6 | GO0003924 | mitofusin 1 | [Swissprot:tr\|O153 |
| | | | | | | | | 23;tr\|O60639;tr\|Q8I |
| | | | | | | | | WA4;tr\|Q9BZB5;tr\| |
| | | | | | | | | Q9NWQ2;] |
| 187 | AGTGCCGTGTG | 1 | 1 | 1 | 0 | GO0003924 | myxovirus | [Swissprot:sp\|P205 |
| | | | | | | | (influenza virus) | 91;tr\|Q8NAA8;tr\|Q |
| | | | | | | | resistance 1, | 96CI3;] |
| | | | | | | | interferon-inducible | |
| | | | | | | | protein p78 | |
| | | | | | | | (mouse) | |
| 188 | CGGAGTCCATT | 7 | 1 | −7 | 1.4 | GO0003924 | neural precursor | [Swissprot:sp\|Q15 |
| | | | | | | | cell expressed, | 019;tr\|Q8IUK9;tr\|Q |
| | | | | | | | developmentally | 96CB0;] |
| | | | | | | | down-regulated 5 | |
| 189 | CAAGCCTTACT | 1 | 0 | −2 | 0.3 | GO0003924 | nucleolar GTPase | [Swissprot:sp\|Q13 |
| | | | | | | | | 823;] |
| 190 | TGGCCCGACGA | 3 | 0 | −6 | 0.9 | GO0003924 | nudix (nucleoside | [Swissprot:sp\|P366 |
| | | | | | | | diphosphate linked | 39;tr\|Q8IV95;] |
| | | | | | | | moiety X)-type | |
| | | | | | | | motif 1 | |
| 191 | ATCCCTTCCCG | 1 | 0 | −2 | 0.3 | GO0003924 | peanut-like 1 | [Swissprot:sp\|Q99 |
| | | | | | | | (Drosophila) | 719;tr\|O95648;tr\|Q |
| | | | | | | | | 96MY5;] |
| 192 | GGGCACAATGC | 1 | 0 | −2 | 0.3 | GO0003924 | peanut-like 1 | [Swissprot:sp\|Q99 |
| | | | | | | | (Drosophila) | 719;tr\|O95648;tr\|Q |
| | | | | | | | | 96MY5;] |
| 193 | GCTAAGGAGAT | 6 | 3 | −2 | 0.46 | GO0003924 | ras-related C3 | [Swissprot:sp\|P151 |
| | | | | | | | botulinum toxin | 54;] |
| | | | | | | | substrate 1 (rho | |
| | | | | | | | family, small GTP | |
| | | | | | | | binding protein | |
| | | | | | | | Rac1) | |
| 194 | TATGACTTAAT | 1 | 2 | 2 | 0.21 | GO0003924 | ras-related C3 | [Swissprot:sp\|P151 |
| | | | | | | | botulinum toxin | 54;] |
| | | | | | | | substrate 1 (rho | |
| | | | | | | | family, small GTP | |
| | | | | | | | binding protein | |
| | | | | | | | Rac1) | |
| 195 | GTTTAATAGAA | 0 | 1 | 2 | 0.3 | GO0003924 | spastic paraplegia | [Swissprot:tr\|O958 |
| | | | | | | | 3A (autosomal | 90;tr\|Q8WXF7;tr\|Q |
| | | | | | | | dominant) | 96FK0;] |
| 196 | TGATATTCCAA | 1 | 0 | −2 | 0.3 | GO0003924 | spastic paraplegia | [Swissprot:tr\|O958 |
| | | | | | | | 3A (autosomal | 90;tr\|Q8WXF7;tr\|Q |
| | | | | | | | dominant) | 96FK0;] |
| 197 | AACTGTACTAC | 1 | 0 | −2 | 0.3 | GO0003924 | v-Ki-ras2 Kirsten | [Swissprot:sp\|P011 |
| | | | | | | | rat sarcoma 2 viral | 18;tr\|Q14014;tr\|Q1 |
| | | | | | | | oncogene homolog | 4015;tr\|Q15285;tr\| |
| | | | | | | | | Q8N2Z2;tr\|Q96D1 |
| | | | | | | | | 0;tr\|Q96FS0;] |
| 198 | GTCACTCTCCC | 1 | 0 | −2 | 0.3 | GO0003924 | v-Ki-ras2 Kirsten | [Swissprot:sp\|P011 |
| | | | | | | | rat sarcoma 2 viral | 18;tr\|Q14014;tr\|Q1 |
| | | | | | | | oncogene homolog | 4015;tr\|Q15285;tr\| |
| | | | | | | | | Q8N2Z2;tr\|Q96D1 |
| | | | | | | | | 0;tr\|Q96FS0;] |
| | | 12 | 2 | −6 | 2.12 | GO0003945 | N-acetyllactos- | 4 matches |
| | | | | | | | amine synthase | |
| | | | | | | | activity | |
| 199 | ATCCGCCACTC | 1 | 0 | −2 | 0.3 | GO0003945 | UDP- | [Swissprot:sp\|P152 |
| | | | | | | | Gal:betaGlcNAc | 91;] |
| | | | | | | | beta 1,4- | |
| | | | | | | | galactosyltransfer- | |
| | | | | | | | ase, polypeptide 1 | |
| 200 | TCCCAGAGACC | 2 | 0 | −4 | 0.6 | GO0003945 | UDP- | [Swissprot:sp\|P152 |
| | | | | | | | Gal:betaGlcNAc | 91;] |
| | | | | | | | beta 1,4- | |
| | | | | | | | galactosyltransfer- | |
| | | | | | | | ase, polypeptide 1 | |
| 201 | GGAGGCAGGTG | 8 | 2 | −4 | 1.18 | GO0003945 | UDP- | [Swissprot:sp\|O60 |
| | | | | | | | Gal:betaGlcNAc | 909;tr\|Q9BUP6;] |
| | | | | | | | beta 1,4- | |
| | | | | | | | galactosyltransfer- | |
| | | | | | | | ase, polypeptide 2 | |
| 202 | GAGAGAAGAGT | 1 | 0 | −2 | 0.3 | GO0003945 | UDP- | [Swissprot:sp\|O60 |
| | | | | | | | Gal:betaGlcNAc | 512;tr\|Q9BPZ4;tr\|Q |
| | | | | | | | beta 1,4- | 9H8T2;] |
| | | | | | | | galactosyltransfer- | |
| | | | | | | | ase, polypeptide 3 | |
| | | 0 | 12 | 24 | 3.62 | GO0004274 | dipeptidyl- | 6 matches |
| | | | | | | | peptidase IV | |
| | | | | | | | activity | |
| 203 | CCATTTAAAGC | 0 | 1 | 2 | 0.3 | GO0004274 | dipeptidylpeptidase | [Swissprot:sp\|P274 |
| | | | | | | | 4 (CD26, | 87;] |
| | | | | | | | adenosine de- | |
| | | | | | | | aminase com- | |
| | | | | | | | plexing protein 2) | |
| 204 | GCTGGGAACCC | 0 | 1 | 2 | 0.3 | GO0004274 | dipeptidylpeptidase | [Swissprot:sp\|P274 |
| | | | | | | | 4 (CD26, | 87;] |
| | | | | | | | adenosine de- | |
| | | | | | | | aminase com- | |
| | | | | | | | plexing protein 2) | |
| 205 | CTCAAAATCAA | 0 | 1 | 2 | 0.3 | GO0004274 | dipeptidylpeptidase | [Swissprot:tr\|Q8IW |
| | | | | | | | 8 | G7;tr\|Q8NEM5;tr\|Q |
| | | | | | | | | 96JX1;tr\|Q9HBM2; |
| | | | | | | | | tr\|Q9HBM3;tr\|Q9H |
| | | | | | | | | BM4;tr\|Q9HBM5;tr\| |
| | | | | | | | | Q9NXF4;] |
| 206 | GGGAAACCCCG | 0 | 7 | 14 | 2.11 | GO0004274 | dipeptidylpeptidase | [Swissprot:tr\|Q8N2 |
| | | | | | | | 9 | J7;tr\|Q8N3F5;tr\|Q8 |
| | | | | | | | | WXD8;tr\|Q96NT8;t |
| | | | | | | | | r\|Q9BVR3;] |
| 207 | GGGGAAACCCC | 0 | 1 | 2 | 0.3 | GO0004274 | dipeptidylpeptidase | [Swissprot:tr\|Q8N2 |
| | | | | | | | 9 | J7;tr\|Q8N3F5;tr\|Q8 |
| | | | | | | | | WXD8;tr\|Q96NT8;t |
| | | | | | | | | r\|Q9BVR3;] |
| 208 | TGTCTGCCTGA | 0 | 1 | 2 | 0.3 | GO0004274 | dipeptidylpeptidase | [Swissprot:tr\|Q8N2 |
| | | | | | | | 9 | J7;tr\|Q8N3F5;tr\|Q8 |
| | | | | | | | | WXD8;tr\|Q96NT8;t |
| | | | | | | | | r\|Q9BVR3;] |
| | | 10 | 1 | −10 | 2.19 | GO0004540 | ribonuclease | 4 matches |
| | | | | | | | activity | |
| 209 | CGCCTGTAGTC | 4 | 0 | −8 | 1.2 | GO0004540 | hypothetical | [Swissprot:tr\|Q8N1 |
| | | | | | | | protein MGC4562 | N8;tr\|Q8TF46;tr\|Q8 |
| | | | | | | | | WTU9;tr\|Q96CM7;] |
| 210 | GACCTTAATGG | 2 | 0 | −4 | 0.6 | GO0004540 | mitotic control | [Swissprot:sp\|Q9Y |
| | | | | | | | protein dis3 | 2L1;] |
| | | | | | | | homolog | |
| 211 | GGACCTGCGCC | 2 | 1 | −2 | 0.2 | GO0004540 | ribonuclease 6 | [Swissprot:sp\|O00 |
| | | | | | | | precursor | 584;tr\|Q8TCU1;tr\| |
| | | | | | | | | Q8T0U2;tr\|Q9NV6 |
| | | | | | | | | 1;tr\|Q9NX85;] |
| 212 | ATACAGCCACT | 2 | 0 | −4 | 0.6 | GO0004540 | ribonuclease H2, | [Swissprot:sp\|O75 |
| | | | | | | | large subunit | 792;] |
| | | 10 | 1 | −10 | 2.19 | GO0005587 | collagen type IV | 3 matches |
| 213 | GACCGCAGGAG | 51 | | −5 | 0.9 | GO0005587 | collagen, type IV, | [Swissprot:sp\|P024 |
| | | | | | | | alpha 1 | 62;tr\|Q8NF88;tr\|Q9 |
| | | | | | | | | NYC5;] |
| 214 | AAGAACCTGTG | 1 | 0 | −2 | 0.3 | GO0005587 | collagen, type IV, | [Swissprot:sp\|P085 |
| | | | | | | | alpha 2 | 72;tr\|Q14052;] |
| 215 | GTGTCAGTTTT | 4 | 0 | −8 | 1.2 | GO0005587 | collagen, type IV, | [Swissprot:sp\|Q14 |
| | | | | | | | alpha 6 | 031;tr\|Q9BS57;] |
| | | 97 | 63 | −1.54 | 2.11 | GO0005859 | muscle myosin | 5 matches |
| 216 | TTCTCACCACC | 4 | 2 | −2 | 0.34 | GO0005859 | myosin light chain | [Swissprot:sp\|P146 |
| | | | | | | | 1 slow a | 49;] |
| 217 | GGGCGGAGCTC | 1 | 0 | −2 | 0.3 | GO0005859 | myosin, light | [Swissprot:sp\|P164 |
| | | | | | | | polypeptide 6, | 75;sp[P24572;] |
| | | | | | | | alkali, smooth | |
| | | | | | | | muscle and non- | |
| | | | | | | | muscle | |
| 218 | GTGCTGAATGG | 72 | 48 | −1.5 | 1.52 | GO0005859 | myosin, light | [Swissprot:sp\|P164 |
| | | | | | | | polypeptide 6, | 75;sp[P24572;] |
| | | | | | | | alkali, smooth | |
| | | | | | | | muscle and non- | |
| | | | | | | | muscle | |
| 219 | GGAGTGTGCTC | 10 | 3 | −3.33 | 1.23 | GO0005859 | myosin, light | [Swissprot:sp\|P248 |
| | | | | | | | polypeptide 9, | 44;tr\|Q9BUF9;] |
| | | | | | | | regulatory | |
| 220 | CCCTTAGCTTT | 10 | 10 | 1 | 0 | GO0005859 | myosin, light | [Swissprot:sp\|P191 |
| | | | | | | | polypeptide, | 05;] |
| | | | | | | | regulatory, non- | |
| | | | | | | | sarcomeric (20 kD) | |
| | | 19 | 41 | 2.16 | 2.37 | GO0006094 | gluconeogenesis | 6 matches |
| 221 | ACTATTTCCAC | 1 | 1 | 1 | 0 | GO0006094 | fructose-1,6- | [Swissprot:sp\|P094 |
| | | | | | | | bisphosphatase 1 | 67;tr\|Q96E46;] |
| 222 | ATCCGCCTGCT | 1 | 0 | −2 | 0.3 | GO0006094 | glucose phosphate | [Swissprot:sp\|P067 |
| | | | | | | | isomerase | 44;tr\|Q9BRD3;] |
| 223 | TAGAAAAATAA | 1 | 1 | 1 | 0 | GO0006094 | glucose phosphate | [Swissprot:sp\|P067 |
| | | | | | | | isomerase | 44;tr\|Q9BRD3;] |
| 224 | TTCATCTCTTG | 0 | 2 | 4 | 0.6 | GO0006094 | pyruvate | [Swissprot:sp\|P114 |
| | | | | | | | carboxylase | 98;] |
| 225 | TCCTCGGGCAG | 1 | 5 | 5 | 0.91 | GO0006094 | solute carrier | [Swissprot:sp\|Q9U |
| | | | | | | | family 25 | BX3;] |
| | | | | | | | (mitochondrial | |
| | | | | | | | carrier; | |
| | | | | | | | dicarboxylate | |
| | | | | | | | transporter), | |
| | | | | | | | member 10 | |
| 226 | TGAGGGAATAA | 15 | 32 | 2.13 | 1.89 | GO0006094 | triosephosphate | [Swissprot:sp\|P009 |
| | | | | | | | isomerase 1 | 38;tr\|Q8WWD0;tr\| |
| | | | | | | | | Q96AG5;] |
| | | 44 | 82 | 1.86 | 3.2 | GO0006469 | negative | 2 matches |
| | | | | | | | regulation of | |
| | | | | | | | protein kinase | |
| | | | | | | | activity | |
| 227 | GAGCTCCACAG | 0 | 2 | 4 | 0.6 | GO0006469 | protein kinase | [Swissprot:sp\|Q9Y |
| | | | | | | | (cAMP-dependent, | 2B9;] |
| | | | | | | | catalytic) inhibitor | |
| | | | | | | | gamma | |
| 228 | TTTCCTCTCAA | 44 | 80 | 1.82 | 2.96 | GO0006469 | stratifin | [Swissprot:sp\|P319 |
| | | | | | | | | 47;tr\|Q96DH0;] |
| | | 12 | 30 | 2.5 | 2.29 | GO0006583 | melanin | 8 matches |
| | | | | | | | biosynthesis from | |
| | | | | | | | tyrosine | |
| 229 | CAACATTCCTG | 0 | 7 | 14 | 2.11 | GO0006583 | D-dopachrome | [Swissprot:sp\|P300 |
| | | | | | | | tautomerase | 46;] |
| 230 | GTGCAGCTGGC | 2 | 0 | −4 | 0.6 | GO0006583 | melanoma antigen | [Swissprot:sp\|Q9U |
| | | | | | | | AIM1 | MX9;] |
| 231 | CCTGGTCAAGA | 7 | 17 | 2.43 | 1.37 | GO0006583 | silver homolog | [Swissprot:sp\|P409 |
| | | | | | | | (mouse | 67;] |
| 232 | GAGAAAGAGGA | 0 | 1 | 2 | 0.3 | GO0006583 | tyrosinase | [Swissprot:sp\|P146 |
| | | | | | | | (oculocutaneous | 79;tr\|Q9UMA2;] |
| | | | | | | | albinism IA) | |
| 233 | TTGGCTGGGCT | 1 | 0 | −2 | 0.3 | GO0006583 | tyrosinase | [Swissprot:sp\|P146 |
| | | | | | | | (oculocutaneous | 79;tr\|Q9UMA2;] |
| | | | | | | | albinism IA) | |
| 234 | AAATATATTTT | 1 | 0 | −2 | 0.3 | GO0006583 | tyrosinase-related | [Swissprot:sp\|P176 |
| | | | | | | | protein 1 | 43;] |
| 235 | CACTATAAAAA | 0 | 2 | 4 | 0.6 | GO0006583 | tyrosinase-related | [Swissprot:sp\|P176 |
| | | | | | | | protein 1 | 43;] |
| 236 | TTTTATACTGC | 1 | 3 | 3 | 0.43 | GO0006583 | tyrosinase-related | [Swissprot:sp\|P176 |
| | | | | | | | protein 1 | 43;] |
| | | 84 | 49 | −1.71 | 2.59 | GO0006887 | exocytosis | 22 matches |
| 237 | CTTTGATCAGG | 2 | 5 | 2.5 | 0.54 | GO0006887 | ADP-ribosylation | [Swissprot:sp\|Q9Y |
| | | | | | | | factor guanine | 6D5;] |
| | | | | | | | nucleotide- | |
| | | | | | | | exchange factor 2 | |
| | | | | | | | (brefeldin A- | |
| | | | | | | | inhibited) | |
| 238 | ACCACAGGGGC | 1 | 0 | −2 | 0.3 | GO0006887 | RAB3D, member | [Swissprot:sp\|O95 |
| | | | | | | | RAS oncogene | 716;] |
| | | | | | | | family | |
| 239 | ACCACAGGGGT | 2 | 0 | −4 | 0.6 | GO0006887 | RAB3D, member | [Swissprot:sp\|O95 |
| | | | | | | | RAS oncogene | 716;] |
| | | | | | | | family | |
| 240 | TTTGAGTTCTG | 2 | 0 | −4 | 0.6 | GO0006887 | SEC10-like 1 (S. | [Swissprot:sp\|O00 |
| | | | | | | | cerevisiae) | 471;tr\|Q8IW24;] |
| 241 | TCTGATATGGT | 0 | 1 | 2 | 0.3 | GO0006887 | SEC15 (S. | [Swissprot:sp\|Q8T |
| | | | | | | | cerevisiae)-like | AG9;tr\|Q9NTA6;tr\| |
| | | | | | | | | Q9NUN4;] |
| 242 | CGGCCCATCTG | 1 | 1 | 1 | 0 | GO0006887 | Sec15B protein | [Swissprot:sp\|Q9Y |
| | | | | | | | | 2D4;tr\|Q9H8D6;] |
| 243 | TTTATTCCTCT | 0 | 1 | 2 | 0.3 | GO0006887 | Sec15B protein | [Swissprot:sp\|Q9Y |
| | | | | | | | | 2D4;tr\|Q9H8D6;] |
| 244 | TGATGATCATT | 1 | 1 | 1 | 0 | GO0006887 | Sec3-like | [Swissprot:sp\|Q9N |
| | | | | | | | | V70;] |
| 245 | GTTTGCGGAGG | 4 | 3 | −1.33 | 0.14 | GO0006887 | brefeldin A- | [Swissprot:sp\|Q9Y |
| | | | | | | | inhibited guanine | 6D6;] |
| | | | | | | | nucleotide- | |
| | | | | | | | exchange protein 1 | |
| 246 | GGCTTTGATTT | 2 | 3 | 1.5 | 0.17 | GO0006887 | coatomer protein | [Swissprot:sp\|P356 |
| | | | | | | | complex, subunit | 06;] |
| | | | | | | | beta 2 (beta prime) | |
| 247 | AATGTTTGTGA | 1 | 0 | −2 | 0.3 | GO0006887 | homolog of yeast | [Swissprot:sp\|Q96 |
| | | | | | | | Sec5 | KP1;] |
| 248 | ATCGATCGCCT | 3 | 2 | −1.5 | 0.16 | GO0006887 | likely ortholog of | [Swissprot:sp\|Q9U |
| | | | | | | | mouse exocyst | PT5;tr\|Q8WV91;tr\| |
| | | | | | | | component protein | Q96BU6;tr\|Q9H9X |
| | | | | | | | 70 kDa homolog | 3;tr\|Q9HA32;] |
| | | | | | | | (S. cerevisiae) | |
| | | | | | | | Exo70: exocyst | |
| | | | | | | | component protein | |
| | | | | | | | 70 kDa homolog | |
| | | | | | | | (S. cerevisiae) | |
| 249 | GGGCCTGGCCT | 2 | 1 | −2 | 0.2 | GO0006887 | likely ortholog of | [Swissprot:sp\|Q9U |
| | | | | | | | mouse exocyst | PT5;tr\|Q8WV91;tr\| |
| | | | | | | | component protein | Q96BU6;tr\|Q9H9X |
| | | | | | | | 70 kDa homolog | 3;tr\|Q9HA32;] |
| | | | | | | | (S. cerevisiae) | |
| | | | | | | | Exo70: exocyst | |
| | | | | | | | component protein | |
| | | | | | | | 70 kDa homolog | |
| | | | | | | | (S. cerevisiae) | |
| 250 | GCGAAGCCCTG | 0 | 1 | 2 | 0.3 | GO0006887 | secretory protein | [Swissprot:sp\|Q96 |
| | | | | | | | SEC8 | A65;tr\|Q8TAR2;] |
| 251 | GAGACCCTGGA | 2 | 2 | 1 | 0 | GO0006887 | similar to S. | [Swissprot:sp\|O60 |
| | | | | | | | cerevisiae Sec6p | 645;] |
| | | | | | | | and R. norvegicus | |
| | | | | | | | rsec6 | |
| 252 | CAGCAGGGGAT | 0 | 1 | 2 | 0.3 | GO0006887 | syntaxin 1A (brain) | [Swissprot:sp\|Q16 |
| | | | | | | | | 623;] |
| 253 | CTCTTAATGTA | 1 | 0 | −2 | 0.3 | GO0006887 | tyrosine 3- | [Swissprot:sp\|P273 |
| | | | | | | | monooxygenase/ | 48;tr\|Q9UP48;] |
| | | | | | | | tryptophan 5- | |
| | | | | | | | monooxygenase | |
| | | | | | | | activation protein, | |
| | | | | | | | theta polypeptide | |
| 254 | GGCCATCTCTT | 30 | 17 | −1.76 | 1.21 | GO0006887 | tyrosine 3-mono- | [Swissprot:sp\|P273 |
| | | | | | | | oxygenase/trypto- | 48;tr\|Q9UP48;] |
| | | | | | | | phan 5-mono- | |
| | | | | | | | oxygenase | |
| | | | | | | | activation protein, | |
| | | | | | | | theta polypeptide | |
| 255 | TGAAAGGGTGT | 1 | 0 | −2 | 0.3 | GO0006887 | tyrosine 3-mono- | [Swissprot:sp\|P273 |
| | | | | | | | oxygenase/trypto- | 48,tr\|Q9UP48,] |
| | | | | | | | phan 5-mono- | |
| | | | | | | | oxygenase | |
| | | | | | | | activation protein, | |
| | | | | | | | theta polypeptide | |
| 256 | TGAGAGGGTGT | 25 | 10 | −2.5 | 1.93 | GO0006887 | tyrosine 3-mono- | [Swissprot:sp\|P273 |
| | | | | | | | oxygenase/trypto- | 48;tr\|Q9UP48;] |
| | | | | | | | phan 5-mono- | |
| | | | | | | | oxygenase | |
| | | | | | | | activation protein, | |
| | | | | | | | theta polypeptide | |
| 257 | AAGAACCAGCG | 1 | 0 | −2 | 0.3 | GO0006887 | vesicie-associated | [Swissprot:sp\|Q15 |
| | | | | | | | membrane protein | 836;tr\|Q9BRV4;] |
| | | | | | | | 3 (cellubrevin) | |
| 258 | TAACCCACTGG | 3 | 0 | −6 | 0.9 | GO0006887 | vesicle-associated | [Swissprot:sp\|Q15 |
| | | | | | | | membrane protein | 836;tr\|Q9BRV4;] |
| | | | | | | | 3 (cellubrevin) | |
| | | 115 | 75 | −1.53 | 2.39 | GO0006979 | response to | 25 matches |
| | | | | | | | oxidative stress | |
| 259 | CCGGGTGATGG | 23 | 19 | −1.21 | 0.26 | GO0006979 | ATX1 antioxidant | [Swissprot:sp\|O00 |
| | | | | | | | protein 1 homolog | 244;] |
| | | | | | | | (yeast) | |
| 260 | CCCGGGAGCGA | 7 | 3 | −2.33 | 0.64 | GO0006979 | PDZ and LIM | [Swissprot:sp\|O00 |
| | | | | | | | domain 1 (elfin) | 151;] |
| 261 | GATGCCGGCAC | 17 | 4 | −4.25 | 2.35 | GO0006979 | angiopoietin-like | [Swissprot:tr\|O438 |
| | | | | | | | factor | 27;] |
| 262 | GCTTAATGTTT | 1 | 1 | 1 | 0 | GO0006979 | catalase | [Swissprot:sp\|P040 |
| | | | | | | | | 40;tr\|Q8TAK2;tr\|Q9 |
| | | | | | | | | BWT9;] |
| 263 | CTTGACATACC | 7 | 8 | 1.14 | 0.1 | GO0006979 | dual specificity | [Swissprot:sp\|P285 |
| | | | | | | | phosphatase 1 | 62;] |
| 264 | GGTGTGAGCCA | 2 | 0 | −4 | 0.6 | GO0006979 | forkhead box M1 | [Swissprot:sp\|Q08 |
| | | | | | | | | 050;] |
| 265 | AACCCTGCCCC | 1 | 0 | −2 | 0.3 | GO0006979 | glutathione | [Swissprot:sp\|P486 |
| | | | | | | | synthetase | 37;] |
| 266 | GTGGGCCTTTG | 4 | 1 | −4 | 0.66 | GO0006979 | methionine sulf- | [Swissprot:sp\|Q9U |
| | | | | | | | oxide reductase A | J68;] |
| 267 | TGGCCCGACGA | 3 | 0 | −6 | 0.9 | GO0006979 | nudix (nucleoside | [Swissprot:sp\|P366 |
| | | | | | | | diphosphate linked | 39;tr\|Q8IV95;] |
| | | | | | | | moiety X)-type | |
| | | | | | | | motif 1 | |
| 268 | TGACAGTGACT | 1 | 0 | −2 | 0.3 | GO0006979 | oxidation | [Swissprot:tr\|Q8N5 |
| | | | | | | | resistance 1 | 73;tr\|Q8N8V0;tr\|Q9 |
| | | | | | | | | H266;tr\|Q9NWC7;] |
| 269 | ACTGCCCCACT | 0 | 1 | 2 | 0.3 | GO0006979 | oxidative-stress | [Swissprot:tr\|O957 |
| | | | | | | | responsive 1 | 47;tr\|Q9UPQ1;] |
| 270 | TTTTCTTCATT | 0 | 2 | 4 | 0.6 | GO0006979 | oxidative-stress | [Swissprot:tr\|O957 |
| | | | | | | | responsive 1 | 47;tr\|Q9UPQ1;] |
| 271 | CCTCCACCTAG | 21 | 14 | −1.5 | 0.61 | GO0006979 | peroxiredoxin 2 | [Swissprot:sp\|P321 |
| | | | | | | | | 19;] |
| 272 | GTGGTACAGGA | 6 | 2 | −3 | 0.74 | GO0006979 | peroxiredoxin 5 | [Swissprot:sp\|P300 |
| | | | | | | | | 44;] |
| 273 | GTGGTGTGTAC | 1 | 1 | 1 | 0 | GO0006979 | scavenger receptor | [Swissprot:tr\|Q9U |
| | | | | | | | class A, member 3 | M15;tr\|Q9UM16;] |
| 274 | TAACTCTCCTG | 0 | 1 | 2 | 0.3 | GO0006979 | scavenger receptor | [Swissprot:tr\|Q9U |
| | | | | | | | class A, member 3 | M15;tr\|Q9UM16;] |
| 275 | AATAAAGCCTT | 6 | 2 | −3 | 0.74 | GO0006979 | selenoprotein P, | [Swissprot:sp\|P499 |
| | | | | | | | plasma, 1 | 08;] |
| 276 | GAGAAATCTAC | 0 | 1 | 2 | 0.3 | GO0006979 | selenoprotein P, | [Swissprot:sp\|P499 |
| | | | | | | | plasma, 1 | 08;] |
| 277 | TCTTTGTTGTT | 6 | 1 | −6 | 1.15 | GO0006979 | selenoprotein P, | [Swissprot:sp\|P499 |
| | | | | | | | plasma, 1 | 08;] |
| 278 | TGTGATAGTAA | 1 | 2 | 2 | 0.21 | GO0006979 | selenoprotein P, | [Swissprot:sp\|P499 |
| | | | | | | | plasma, 1 | 08;] |
| 279 | ATGGCCATAGA | 3 | 8 | 2.67 | 0.84 | GO0006979 | serine/threonine | [Swissprot:sp\|O00 |
| | | | | | | | kinase 25 (STE20 | 506;tr\|Q96BA2;] |
| | | | | | | | homolog, yeast) | |
| 280 | AAAAAGCAGAT | 3 | 2 | −1.5 | 0.16 | GO0006979 | superoxide dis- | [Swissprot:sp\|P004 |
| | | | | | | | mutase 1, soluble | 41;] |
| | | | | | | | (amyotrophic | |
| | | | | | | | lateral sclerosis 1 | |
| | | | | | | | (adult)) | |
| 281 | ACATTTCCTGT | 1 | 0 | −2 | 0.3 | GO0006979 | superoxide dis- | [Swissprot:sp\|P004 |
| | | | | | | | mutase 1, soluble | 41;] |
| | | | | | | | (amyotrophic | |
| | | | | | | | lateral sclerosis 1 | |
| | | | | | | | (adult)) | |
| 282 | CAGGCCTTCAG | 0 | 1 | 2 | 0.3 | GO0006979 | superoxide dis- | [Swissprot:sp\|P004 |
| | | | | | | | mutase 1, soluble | 41;] |
| | | | | | | | (amyotrophic | |
| | | | | | | | lateral sclerosis 1 | |
| | | | | | | | (adult)) | |
| 283 | GCTTGCAAAAA | 1 | 1 | 1 | 0 | GO0006979 | superoxide dis- | [Swissprot:sp\|P041 |
| | | | | | | | mutase 2, | 79;tr\|Q96AM7;tr\|Q |
| | | | | | | | mitochondrial | 96EE6;tr\|Q9UG59;] |
| | | 25 | 47 | 1.88 | 2.04 | GO0009306 | protein secretion | 23 matches |
| 284 | ATTAACAAAGC | 3 | 8 | 2.67 | 0.84 | GO0009306 | GNAS complex | [Swissprot:sp\|P048 |
| | | | | | | | locus | 95;tr\|O60726;tr\|O7 |
| | | | | | | | | 5632;tr\|O75633;tr\| |
| | | | | | | | | O75684;tr\|O95467; |
| | | | | | | | | tr\|Q14455;tr\|Q8TB |
| | | | | | | | | C0;tr\|Q96H70;] |
| 285 | AAGCAAACTAA | 0 | 1 | 2 | 0.3 | GO0009306 | calnexin | [Swissprot:sp\|P278 |
| | | | | | | | | 24;] |
| 286 | CCTCACTTTCT | 0 | 1 | 2 | 0.3 | GO0009306 | calnexin | [Swissprot:sp\|P278 |
| | | | | | | | | 24;] |
| 287 | CCTCACTTTTT | 0 | 1 | 2 | 0.3 | GO0009306 | calnexin | [Swissprot:sp\|P278 |
| | | | | | | | | 24;] |
| 288 | CGGGATGCAGA | 0 | 1 | 2 | 0.3 | GO0009306 | calnexin | [Swissprot:sp\|P278 |
| | | | | | | | | 24;] |
| 289 | TAACAGTTGTG | 0 | 4 | 8 | 1.21 | GO0009306 | calnexin | [Swissprot:sp\|P278 |
| | | | | | | | | 24;] |
| 290 | TTACTAAATGG | 2 | 3 | 1.5 | 0.17 | GO0009306 | calnexin | [Swissprot:sp\|P278 |
| | | | | | | | | 24;] |
| 291 | GTGGAATAAAG | 5 | 7 | 1.4 | 0.24 | GO0009306 | latent transforming | [Swissprot:tr\|Q147 |
| | | | | | | | growth factor beta | 67;] |
| | | | | | | | binding protein 2 | |
| 292 | GCGAAACCCTG | 5 | 5 | 1 | 0 | GO0009306 | polymeric | [Swissprot:sp\|P018 |
| | | | | | | | immunoglobulin | 33;tr\|Q8IZY7;] |
| | | | | | | | receptor | |
| 293 | AAGTGAAACAC | 1 | 1 | 1 | 0 | GO0009306 | protein disulfide | [Swissprot:sp\|P136 |
| | | | | | | | isomerase related | 67;] |
| | | | | | | | protein (calcium- | |
| | | | | | | | binding protein, | |
| | | | | | | | intestinal-related) | |
| 294 | ATCCAGGGTCC | 2 | 1 | −2 | 0.2 | GO0009306 | protein disulfide | [Swissprot:sp\|P136 |
| | | | | | | | isomerase related | 67;] |
| | | | | | | | protein (calcium- | |
| | | | | | | | binding protein, | |
| | | | | | | | intestinal-related) | |
| 295 | GACACTTGGGG | 1 | 0 | −2 | 0.3 | GO0009306 | protein transport | [Swissprot:sp\|P383 |
| | | | | | | | protein SEC61 | 78;sp\|Q9Y2R3;tr\|Q |
| | | | | | | | alpha subunit | 8N0Z4;tr\|Q8N3U3;tr\| |
| | | | | | | | isoform 1 | Q8NC71;tr\|Q9BU |
| | | | | | | | | 16;] |
| 296 | GTTCTCCCACT | 2 | 3 | 1.5 | 0.17 | GO0009306 | protein transport | [Swissprot:sp\|P383 |
| | | | | | | | protein SEC61 | 78;sp\|Q9Y2R3;tr\|Q |
| | | | | | | | alpha subunit | 8N0Z4;tr\|Q8N3U3;tr\| |
| | | | | | | | isoform 1 | Q8NC71;tr\|Q9BU |
| | | | | | | | | 16;] |
| 297 | TTTATGTCTGG | 0 | 1 | 2 | 0.3 | GO0009306 | protein transport | [Swissprot:sp\|P383 |
| | | | | | | | protein SEC61 | 78;sp\|Q9Y2R3;tr\|Q |
| | | | | | | | alpha subunit | 8N0Z4;tr\|Q8N3U3;tr\| |
| | | | | | | | isoform 1 | Q8NC71;tr\|Q9BU |
| | | | | | | | | 16;] |
| 298 | CAGAAAAAAGC | 0 | 1 | 2 | 0.3 | GO0009306 | syntaxin binding | [Swissprot:sp\|Q64 |
| | | | | | | | protein 1 | 320;tr\|Q96TG8;] |
| 299 | CTTCAGGACCT | 1 | 1 | 1 | 0 | GO0009306 | syntaxin binding | [Swissprot:sp\|Q64 |
| | | | | | | | protein 1 | 320;tr\|Q96TG8;] |
| 300 | TCAGAGATGAG | 0 | 1 | 2 | 0.3 | GO0009306 | syntaxin binding | [Swissprot:sp\|Q15 |
| | | | | | | | protein 2 | 833;tr\|O00184;tr\|Q |
| | | | | | | | | 9BU65;] |
| 301 | AACATTCTAAG | 1 | 1 | 1 | 0 | GO0009306 | syntaxin binding | [Swissprot:sp\|O00 |
| | | | | | | | protein 3 | 186;tr\|Q9UPD7;] |
| 302 | GGAATACAGAA | 0 | 1 | 2 | 0.3 | GO0009306 | vacuolar protein | [Swissprot:sp\|Q96 |
| | | | | | | | sorting 33A (yeast) | AX1;tr\|Q9H6C4;] |
| 303 | TCTGGACTTTT | 1 | 0 | −2 | 0.3 | GO0009306 | vacuolar protein | [Swissprot:sp\|Q96 |
| | | | | | | | sorting 33A (yeast) | AX1;tr\|Q9H6C4;] |
| 304 | CTGCTAAGATG | 0 | 3 | 6 | 0.91 | GO0009306 | vacuolar protein | [Swissprot:sp\|Q9H |
| | | | | | | | sorting 33B (yeast) | 267;] |
| 305 | TATGACCACAA | 1 | 1 | 1 | 0 | GO0009306 | vacuolar protein | [Swissprot:sp\|Q9N |
| | | | | | | | sorting 45A (yeast) | RW7;] |
| 306 | AATACAGGATC | 0 | 1 | 2 | 0.3 | GO0009306 | vesicle transport- | [Swissprot:tr\|O607 |
| | | | | | | | related protein | 54;tr\|O94990;tr\|Q8 |
| | | | | | | | | WVM8;tr\|Q9BZI3;tr\| |
| | | | | | | | | Q9UNL3;tr\|Q9Y6A |
| | | | | | | | | 8;] |
| | | 16 | 4 | −4 | 2.13 | GO0015036 | disulfide | 9 matches |
| | | | | | | | oxidoreductase | |
| | | | | | | | activity | |
| 307 | GCTGGAGCTAG | 2 | 1 | −2 | 0.2 | GO0015036 | dihydrolipoamide | [Swissprot:sp\|P096 |
| | | | | | | | dehydrogenase | 22;tr\|Q8WTS4;] |
| | | | | | | | (E3 component of | |
| | | | | | | | pyruvate | |
| | | | | | | | dehydrogenase | |
| | | | | | | | complex, 2-oxo- | |
| | | | | | | | glutarate complex, | |
| | | | | | | | branched chain | |
| | | | | | | | keto acid | |
| | | | | | | | dehydrogenase | |
| | | | | | | | complex) | |
| 308 | GCATCTTCAAT | 1 | 0 | −2 | 0.3 | GO0015036 | dihydropyrimidine | [Swissprot:sp\|Q12 |
| | | | | | | | dehydrogenase | 882;tr\|Q96HL6;tr\|Q |
| | | | | | | | | 96TH1;] |
| 309 | CTGCTGCACTC | 5 | 1 | −5 | 0.9 | GO0015036 | glutathione | [Swissprot:sp\|P003 |
| | | | | | | | reductase | 90;] |
| 310 | AGACGCACTCT | 1 | 2 | 2 | 0.21 | GO0015036 | hypothetical | [Swissprot:tr\|Q8IW |
| | | | | | | | protein FLJ23322 | F2;tr\|Q8N378;tr\|Q9 |
| | | | | | | | | 6BD1;tr\|Q9H5L5;tr\| |
| | | | | | | | | Q9H6M8;] |
| 311 | TTAGACATTAC | 1 | 0 | −2 | 0.3 | GO0015036 | hypothetical | [Swissprot:tr\|Q8N1 |
| | | | | | | | protein FLJ30473 | V3;tr\|Q8N5E0;tr\|Q |
| | | | | | | | | 96NN9;] |
| 312 | CCGTTTAGCAG | 1 | 0 | −2 | 0.3 | GO0015036 | succinate | [Swissprot:sp\|P310 |
| | | | | | | | dehydrogenase | 40;tr\|Q8IW48;] |
| | | | | | | | complex, subunit | |
| | | | | | | | A, flavoprotein (Fp) | |
| 313 | TCATAACTGTC | 2 | 0 | −4 | 0.6 | GO0015036 | succinate | [Swissprot:sp\|P310 |
| | | | | | | | dehydrogenase | 40;tr\|Q8IW48;] |
| | | | | | | | complex, subunit | |
| | | | | | | | A, flavoprotein (Fp) | |
| 314 | GGTTCCCTGAG | 1 | 0 | −2 | 0.3 | GO0015036 | thioredoxin | [Swissprot:sp\|Q16 |
| | | | | | | | reductase 1 | 881;tr\|Q99475;tr\|Q |
| | | | | | | | | 9UES8;] |
| 315 | TCCGAGCCCCC | 2 | 0 | −4 | 0.6 | GO0015036 | thioredoxin | [Swissprot:tr\|Q9NN |
| | | | | | | | reductase 2 | W7;] |
| | | 24 | 53 | 2.21 | 3.07 | GO0016272 | prefoldin complex | 6 matches |
| 316 | AATTAATTGTA | 1 | 1 | 1 | 0 | GO0016272 | chromosome 19 | [Swissprot:tr\|Q8TC |
| | | | | | | | open reading | 23;tr\|Q96C15;tr\|Q9 |
| | | | | | | | frame 2 | UNU3;] |
| 317 | AGGCTTTAGGG | 0 | 1 | 2 | 0.3 | GO0016272 | chromosome 19 | [Swissprot:tr\|Q8TC |
| | | | | | | | open reading | 23;tr\|Q96C15;tr\|Q9 |
| | | | | | | | frame 2 | UNU3;] |
| 318 | GGAGAAGATGA | 2 | 6 | 3 | 0.75 | GO0016272 | prefoldin 2 | [Swissprot:sp\|Q9U |
| | | | | | | | | HV9;tr\|O95334;] |
| 319 | GAAATGATGAG | 18 | 25 | 1.39 | 0.55 | GO0016272 | prefoldin 5 | [Swissprot:sp\|Q99 |
| | | | | | | | | 471;tr\|Q9C083;tr\|Q |
| | | | | | | | | 9C084;] |
| 320 | TTGCTAGAGGG | 3 | 17 | 5.67 | 2.84 | GO0016272 | ubiquitously- | [Swissprot:sp\|Q9U |
| | | | | | | | expressed | BK9;tr\|Q9Y6E5;] |
| | | | | | | | transcript | |
| 321 | AAATTAAAACA | 0 | 3 | 6 | 0.91 | GO0016272 | von Hippel-Lindau | [Swissprot:sp\|Q15 |
| | | | | | | | binding protein 1 | 765;] |
| | | 27 | 10 | −2.7 | 2.28 | GO0016758 | transferase, | 9 matches |
| | | | | | | | transferring | |
| | | | | | | | hexosyl groups | |
| | | | | | | | activity | |
| 322 | GCCTGTTTGGG | 4 | 0 | −8 | 1.2 | GO0016758 | UDP glycosyl- | [Swissprot:sp\|P192 |
| | | | | | | | transferase 1 | 24;tr\|Q8WUQ4;] |
| | | | | | | | family, polypeptide | |
| | | | | | | | A6 | |
| 323 | CTAAAATGCTT | 1 | 0 | −2 | 0.3 | GO0016758 | glycogenin | [Swissprot:sp\|P469 |
| | | | | | | | | 76;tr\|Q8N5Y3;] |
| 324 | GAAAAAGATGT | 0 | 1 | 2 | 0.3 | GO0016758 | glycosyltransferase | [Swissprot:tr\|Q8N2 |
| | | | | | | | AD-017 | J6;tr\|Q9P0I5;] |
| 325 | GGAAATATTCC | 1 | 0 | −2 | 0.3 | GO0016758 | gycosyltransferase | [Swissprot:tr\|Q96K |
| | | | | | | | | A2;tr\|Q9H1C3;] |
| 326 | AGTGAGGATAG | 6 | 1 | −6 | 1.15 | GO0016758 | hypothetical | [Swissprot:tr\|Q8NA |
| | | | | | | | protein FLJ35155 | L3;tr\|Q8NBI6;tr\|Q8 |
| | | | | | | | | WV03;tr\|Q96ME0;] |
| 327 | CAGGAGAACTG | 2 | 0 | −4 | 0.6 | GO0016758 | hypothetical | [Swissprot:tr\|Q8NA |
| | | | | | | | protein FLJ35155 | L3;tr\|Q8NBI6;tr\|Q8 |
| | | | | | | | | WV03;tr\|Q96ME0;] |
| 328 | GGGCTGCTGCC | 10 | 5 | −2 | 0.67 | GO0016758 | hypothetical | [Swissprot:tr\|Q8N3 |
| | | | | | | | protein FLJ35207 | Y3;tr\|Q8N8Y6;tr\|Q |
| | | | | | | | | 8NAK3;tr\|Q8WY62;] |
| 329 | GAGACTGTAGG | 1 | 0 | −2 | 0.3 | GO0016758 | hypothetical | [Swissprot:tr\|Q8NB |
| | | | | | | | protein | P2;] |
| | | | | | | | LOC167127 | |
| 330 | TGAACCCGCCA | 2 | 3 | 1.5 | 0.17 | GO0016758 | mannosyl (alpha- | [Swissprot:tr\|Q96G |
| | | | | | | | 1,3-)-glycoprotein | H4;tr\|Q9NSK6;tr\|Q |
| | | | | | | | beta-1,4-N- | 9UQ53;] |
| | | | | | | | acetylglucosaminyl | |
| | | | | | | | transferase, | |
| | | | | | | | isoenzyme B | |
| | | 9 | 0 | −18 | 2.7 | GO0019717 | synaptosome | 4 matches |
| 331 | AAAACTGGGGA | 1 | 0 | −2 | 0.3 | GO0019717 | vesicle-associated | [Swissprot:sp\|P190 |
| | | | | | | | membrane protein | 65;] |
| | | | | | | | 2 (synaptobrevin 2) | |
| 332 | CCCCCAATTCT | 4 | 0 | −8 | 1.2 | GO0019717 | vesicle-associated | [Swissprot:sp\|P190 |
| | | | | | | | membrane protein | 65;] |
| | | | | | | | 2 (synaptobrevin 2) | |
| 333 | AAGAACCAGCG | 1 | 0 | −2 | 0.3 | GO0019717 | vesicle-associated | [Swissprot:sp\|Q15 |
| | | | | | | | membrane protein | 836;tr\|Q9BRV4;] |
| | | | | | | | 3 (cellubrevin) | |
| 334 | TAACCCACTGG | 3 | 0 | −6 | 0.9 | GO0019717 | vesicle-associated | [Swissprot:sp\|Q15 |
| | | | | | | | membrane protein | 836;tr\|Q9BRV4;] |
| | | | | | | | 3 (cellubrevin) | |
| | | 16 | 37 | 2.31 | 2.44 | GO0019992 | diacylglycerol | 14 matches |
| | | | | | | | binding activity | |
| 335 | CAGCTGAGGGC | 0 | 1 | 2 | 0.3 | GO0019992 | RAS guanyl | [Swissprot:tr\|Q9UL |
| | | | | | | | releasing protein 2 | 65;] |
| | | | | | | | (calcium and DAG- | |
| | | | | | | | regulated) | |
| 336 | CGCACACACAT | 1 | 2 | 2 | 0.21 | GO0019992 | diacylglycerol | [Swissprot:sp\|P237 |
| | | | | | | | kinase, alpha | 43;tr\|O75484;tr\|O9 |
| | | | | | | | 8O kDa | 5217;tr\|Q8IZ56;tr\|Q |
| | | | | | | | | 8N5Q2;] |
| 337 | AGGGCAAGGCC | 0 | 2 | 4 | 0.6 | GO0019992 | diacylglycerol | [Swissprot:sp\|Q13 |
| | | | | | | | kinase, zeta | 574;tr\|Q8IVW9;] |
| | | | | | | | 104 kDa | |
| 338 | TTTACAGCTGG | 5 | 7 | 1.4 | 0.24 | GO0019992 | diacylglycerol | [Swissprot:sp\|Q13 |
| | | | | | | | kinase, zeta | 574;tr\|Q8IVWN9;] |
| | | | | | | | 104 kDa | |
| 339 | CTTTAAAATAT | 0 | 1 | 2 | 0.3 | GO0019992 | protein kinase C, | [Swissprot:sp\|P057 |
| | | | | | | | beta 1 | 71;] |
| 340 | GGGGACTGGTG | 0 | 2 | 4 | 0.6 | GO0019992 | protein kinase C, | [Swissprot:sp\|Q05 |
| | | | | | | | delta | 655;] |
| 341 | GTACTTCCTCT | 0 | 1 | 2 | 0.3 | GO0019992 | protein kinase C, | [Swissprot:sp\|Q05 |
| | | | | | | | delta | 655;] |
| 342 | TCAGTGACCAG | 1 | 4 | 4 | 0.66 | GO0019992 | protein kinase C, | [Swissprot:sp\|P247 |
| | | | | | | | eta | 23;tr\|Q8NE03;tr\|Q9 |
| | | | | | | | | BVQ0;] |
| 343 | TGAAAACCTGA | 1 | 0 | −2 | 0.3 | GO0019992 | protein kinase C, | [Swissprot:sp\|O94 |
| | | | | | | | nu | 806;tr\|Q15451;tr\|Q |
| | | | | | | | | 8NEL8;] |
| 344 | CGGTTTCCAAG | 1 | 3 | 3 | 0.43 | GO0019992 | protein kinase C, | [Swissprot:sp\|Q05 |
| | | | | | | | zeta | 513;] |
| 345 | GCCTTGATCTC | 3 | 3 | 1 | 0 | GO0019992 | protein kinase D2 | [Swissprot:sp\|Q9B |
| | | | | | | | | ZL6;tr\|Q8N2H2;tr\| |
| | | | | | | | | Q8NCK8;] |
| 346 | TGGATTTTGGG | 2 | 3 | 1.5 | 0.17 | GO0019992 | v-raf murine | [Swissprot:sp\|P103 |
| | | | | | | | sarcoma 3611 viral | 98;tr\|O96II5;] |
| | | | | | | | oncogene homolog | |
| | | | | | | | 1 | |
| 347 | TGTATACAAGG | 0 | 5 | 10 | 1.51 | GO0019992 | v-raf-1 murine | [Swissprot:sp\|P040 |
| | | | | | | | leukemia viral | 49;] |
| | | | | | | | oncogene homolog | |
| | | | | | | | 1 | |
| 348 | GGCCTGGGGGT | 2 | 3 | 1.5 | 0.17 | GO0019992 | vav 3 oncogene | [Swissprot:sp\|Q9U |
| | | | | | | | | KW4;tr\|O60498;] |
| | | 0 | 9 | 18 | 2.72 | GO0030089 | phycobilisome | 3 matches |
| 349 | GTTGCTGTCCC | 0 | 1 | 2 | 0.3 | GO0030089 | hypothetical | [Swissprot:tr\|Q9BU |
| | | | | | | | protein MGC4293 | 89;] |
| 350 | TATGAGCACGA | 0 | 3 | 6 | 0.91 | GO0030089 | hypothetical | [Swissprot:tr\|Q9BU |
| | | | | | | | protein MGC4293 | 89;] |
| 351 | ACATCATACTG | 0 | 5 | 10 | 1.51 | GO0030089 | importin 4 | [Swissprot:tr\|Q8NC |
| | | | | | | | | G8 |

TABLE 8

| | | | | | |
|---|---|---|---|---|---|
| Tags | Ana | Kata | Ratio | Significance | Pattern/description |

| | | | | | | | |
|---|---|---|---|---|---|---|---|
| | | 10 | 1 | −10 | 2.19 | ME: GLA1 | 2 matches |
| 352 | TTCTCTCCACA | 1 | 0 | −2 | 0.3 | ME: GLA1 bone gamma- | Swissprot: |
| | | | | | | carboxyglutamate (gla) protein | sp\|P02818] |
| | | | | | | (osteocalcin) | |
| 353 | GTTTATGGATA | 9 | 1 | −9 | 1.92 | ME: GLA1 matrix Gla protein | Swissprot: |
| | | | | | | | sp\|P08493] |
| | | 7 | 22 | 3.14 | 2.3 | ME: PARKIN_FINGER3 | 14 matches |
| 354 | CCTGGCAGTCA | 0 | 1 | 2 | 0.3 | ME: PARKIN_FINGER3 | Swissprot: |
| | | | | | | KIAA0708 protein | tr\|O75188] |
| 355 | ATCTGTCACTT | 0 | 2 | 4 | 0.6 | ME: PARKIN_FINGER3 | Swissprot: |
| | | | | | | TRIAD3 protein | sp\|Q9NWF9] |
| 356 | AAGCCTTGCTG | 1 | 5 | 5 | 0.91 | ME: PARKIN_FINGER3 | Swissprot: |
| | | | | | | ariadne homolog 2 | sp\|O95376] |
| | | | | | | (Drosophila) | |
| 357 | ATGTCAACCAA | 0 | 1 | 2 | 0.3 | ME: PARKIN_FINGER3 | Swissprot: |
| | | | | | | ariadne homolog 2 | sp\|O95376] |
| | | | | | | (Drosophila) | |
| 358 | TCTGTGGCTCA | 0 | 1 | 2 | 0.3 | ME: PARKIN_FINGER3 | Swissprot: |
| | | | | | | (Drosophila) | |
| 359 | TTGAACTGGCC | 2 | 0 | −4 | 0.6 | ME: PARKIN_FINGER3 | Swissprot: |
| | | | | | | ariadne homolog 2 | sp\|O95376] |
| | | | | | | (Drosophila) | |
| 360 | ATTAGGAACTG | 0 | 1 | 2 | 0.3 | ME: PARKIN_FINGER3 | Swissprot: |
| | | | | | | ariadne homolog, ubiquitin- | sp\|Q9Y4X5] |
| | | | | | | conjugating enzyme E2 binding | |
| | | | | | | protein, 1 (Drosophila) | |
| 361 | GACAAAGCAAG | 0 | 1 | 2 | 0.3 | ME: PARKIN_FINGER3 | Swissprot: |
| | | | | | | ariadne homolog, ubiquitin- | sp\|Q9Y4X5] |
| | | | | | | conjugating enzyme E2 binding | |
| | | | | | | protein, 1 (Drosophila) | |
| 362 | CTGACCCAGCC | 2 | 2 | 1 | 0 | ME: PARKIN_FINGER3 | Swissprot: |
| | | | | | | chromosome 20 open reading | sp\|Q9BYM8] |
| | | | | | | frame 18 | |
| 363 | GTGCAAAATGG | 0 | 1 | 2 | 0.3 | ME: PARKIN_FINGER3 | Swissprot: |
| | | | | | | frame 18 | |
| 364 | CTCAGGAGAGA | 0 | 2 | 4 | 0.6 | ME: PARKIN_FINGER3 | Swissprot: |
| | | | | | | hypothetical protein | tr\|Q9NTD7] |
| | | | | | | DKFZP434A0225 | |
| 365 | GCCTGCTCCCT | 1 | 4 | 4 | 0.66 | ME: PARKIN_FINGER3 | Swissprot: |
| | | | | | | hypothetical protein FLJ10111 | tr\|Q96EP0] |
| 366 | TATACGTTATG | 0 | 1 | 2 | 0.3 | ME: PARKIN_FINGER3 ring | Swissprot: |
| | | | | | | finger protein 144 | sp\|P50876] |
| 367 | GGCTGCAGTCT | 1 | 0 | −2 | 0.3 | ME: PARKIN_FINGER3 ring | Swissprot: |
| | | | | | | finger protein 19 | sp\|Q9NV58] |
| | | 7 | 22 | 3.14 | 2.3 | ME: PARKIN_TRIAD | 14 matches |
| 368 | CCTGGCAGTCA | 0 | 1 | 2 | 0.3 | ME: PARKIN_TRIAD KIAA0708 | Swissprot: |
| | | | | | | protein | tr\|O75188] |
| 369 | ATCTGTCACTT | 0 | 2 | 4 | 0.6 | ME: PARKIN_TRIAD TRIAD3 | Swissprot: |
| | | | | | | protein | sp\|Q9NWF9] |
| 370 | AAGCCTTGCTG | 1 | 5 | 5 | 0.91 | ME: PARKIN_TRIAD ariadne | Swissprot: |
| | | | | | | homolog 2 (Drosophila) | sp\|O95376] |
| 371 | ATGTCAACCAA | 0 | 1 | 2 | 0.3 | ME: PARKIN_TRIAD ariadne | Swissprot: |
| | | | | | | homolog 2 (Drosophila) | sp\|O95376] |
| 372 | TCTGTGGCTCA | 0 | 1 | 2 | 0.3 | ME: PARKIN_TRIAD ariadne | Swissprot: |
| | | | | | | homolog 2 (Drosophila) | sp\|O95376] |
| 373 | TTGAACTGGCC | 2 | 0 | −4 | 0.6 | ME: PARKIN_TRIAD ariadne | Swissprot: |
| | | | | | | homolog 2 (Drosophila) | sp\|O95376] |
| 374 | ATTAGGAACTG | 0 | 1 | 2 | 0.3 | ME: PARKIN_TRIAD ariadne | Swissprot: |
| | | | | | | homolog, ubiquitin-conjugating | sp\|Q9Y4X5] |
| | | | | | | enzyme E2 binding protein, 1 | |
| | | | | | | (Drosophila) | |
| 375 | GACAAAGCAAG | 0 | 1 | 2 | 0.3 | ME: PARKIN_TRIAD ariadne | Swissprot: |
| | | | | | | homolog, ubiquitin-conjugating | sp\|Q9Y4X5] |
| | | | | | | enzyme E2 binding protein, 1 | |
| | | | | | | (Drosophila) | |
| 376 | CTGACCCAGCC | 2 | 2 | 1 | 0 | ME: PARKIN_TRIAD | Swissprot: |
| | | | | | | chromosome 20 open reading | sp\|Q9BYM8] |
| | | | | | | frame 18 | |
| 377 | GTGCAAAATGG | 0 | 1 | 2 | 0.3 | ME: PARKIN_TRIAD | Swissprot: |
| | | | | | | chromosome 20 open reading | sp\|Q9BYM8] |
| | | | | | | frame 18 | |
| 378 | CTCAGGAGAGA | 0 | 2 | 4 | 0.6 | ME: PARKIN_TRIAD | Swissprot: |
| | | | | | | hypothetical protein | tr\|Q9NTD7] |
| | | | | | | DKFZP434A0225 | |
| 379 | GCCTGCTCCCT | 1 | 4 | 4 | 0.66 | ME: PARKIN_TRIAD | Swissprot: |
| | | | | | | hypothetical protein FLJ10111 | tr\|Q96EP0] |
| 380 | TATACGTTATG | 0 | 1 | 2 | 0.3 | ME: PARKIN_TRIAD ring finger | Swissprot: |
| | | | | | | protein 144 | sp\|P50876] |
| 381 | GGCTGCAGTCT | 1 | 0 | −2 | 0.3 | ME: PARKIN_TRIAD ring finger | Swissprot: |
| | | | | | | protein 19 | sp\|Q9NV58] |
| | | 10 | 1 | −10 | 2.19 | PF: C4 | 3 matches |
| 382 | GACCGCAGGAG | 5 | 1 | −5 | 0.9 | PF: C4 collagen, type IV, | Swissprot: |
| | | | | | | alpha 1 | sp\|P02462] |
| 383 | AAGAACCTGTG | 1 | 0 | −2 | 0.3 | PF: C4 collagen, type IV, | Swissprot: |
| | | | | | | alpha 2 | sp\|P08572] |
| 384 | GTGTCAGTTTT | 4 | 0 | −8 | 1.2 | PF: C4 collagen, type IV, | Swissprot: |
| | | | | | | alpha 6 | sp\|Q14031] |
| | | 38 | 16 | −2.38 | 2.55 | PF: CADHERIN_C_TERM | 8 matches |
| 385 | GTTGTCATCAC | 1 | 0 | −2 | 0.3 | PF: CADHERIN_C_TERM | Swissprot: |
| | | | | | | (Manual) Desmoglein, intemal | sp\|Q02413] |
| | | | | | | tag | |
| 386 | TGTGGGTGCTG | 15 | 5 | −3 | 1.56 | PF: CADHERIN_C_TERM | Swissprot: |
| | | | | | | cadherin 1, type 1, E-cadherin | sp\|P12830] |
| | | | | | | (epithelial) | |
| 387 | CCTAGACCTGG | 0 | 1 | 2 | 0.3 | PF: CADHERIN_C_TERM | Swissprot: |
| | | | | | | cadherin 11 type 2, OB- | sp\|P55287] |
| | | | | | | cadherin (osteoblast) | |
| 388 | AGCACCCACCC | 0 | 1 | 2 | 0.3 | PF: CADHERIN_C_TERM | Swissprot: |
| | | | | | | cadherin 4, type 1, R-cadherin | sp\|P55283] |
| | | | | | | (retinal) | |
| 389 | GCCTCAGCCTC | 0 | 1 | 2 | 0.3 | PF: CADHERIN_C_TERM | Swissprot: |
| | | | | | | cadherin-like 24 | tr\|Q9H6Y4] |
| 390 | CAGGAGTGTGC | 17 | 5 | −3.4 | 1.96 | PF: CADHERIN_C_TERM | Swissprot: |
| | | | | | | desmocollin 3 | sp\|Q14574] |
| 391 | TATGCCCGAAT | 3 | 2 | −1.5 | 0.16 | PF: CADHERIN_C_TERM | Swissprot: |
| | | | | | | desmocollin 3 | sp\|Q14574] |
| 392 | TAACTGGCCTT | 2 | 1 | −2 | 0.2 | PF: CADHERIN_C_TERM | Swissprot: |
| | | | | | | desmoglein 1 | sp\|Q02413] |
| | | 0 | 12 | 24 | 3.62 | PF: DPPIV_N_TERM | 6 matches |
| 393 | CCATTTAAAGC | 0 | 1 | 2 | 0.3 | PF: DPPIV_N_TERM | Swissprot: |
| | | | | | | dipeptidylpeptidase 4 (CD26, | sp\|P27487] |
| | | | | | | adenosine deaminase | |
| | | | | | | complexing protein 2) | |
| 394 | GCTGGGAACCC | 0 | 1 | 2 | 0.3 | PF: DPPIV_N_TERM | Swissprot: |
| | | | | | | dipeptidylpeptidase 4 (CD26, | sp\|P27487] |
| | | | | | | adenosine deaminase | |
| | | | | | | complexing protein 2) | |
| 395 | CTCAAAATCAA | 0 | 1 | 2 | 0.3 | PF: DPPIV_N_TERM | Swissprot: |
| | | | | | | dipeptidylpeptidase 8 | tr\|Q8IWG7] |
| 396 | GGGAAACCCCG | 0 | 7 | 14 | 2.11 | PF: DPPIV_N_TERM | Swissprot: |
| | | | | | | dipeptidylpeptidase 9 | tr\|Q8N2J7] |
| 397 | GGGGAAACCCC | 0 | 1 | 2 | 0.3 | PF: DPPIV_N_TERM | Swissprot: |
| | | | | | | dipeptidylpeptidase 9 | tr\|Q8N2J7] |
| 398 | TGTCTGCCTGA | 0 | 1 | 2 | 0.3 | PF: DPPIV_N_TERM | Swissprot: |
| | | | | | | dipeptidylpeptidase 9 | tr\|Q8N2J7] |
| | | 5 | 18 | 3.6 | 2.19 | PF: GRAM | 9 matches |
| 399 | GGGCTGCTCTT | 2 | 2 | 1 | 0 | PF: GRAM KIAA0676 | Swissprot: |
| | | | | | | protein | tr\|O75163] |
| 400 | CGACAGCGTTC | 0 | 1 | 2 | 0.3 | PF: GRAM KIAA0767 | Swissprot: |
| | | | | | | protein | tr\|Q9Y4B9] |
| 401 | TCCTATCCCAG | 1 | 0 | −2 | 0.3 | PF: GRAM KIAA0767 | Swissprot: |
| | | | | | | protein | tr\|Q9Y4B9] |
| 402 | GAAGTACAGTA | 0 | 1 | 2 | 0.3 | PF: GRAM KIAA1201 | Swissprot: |
| | | | | | | protein | tr\|Q9ULL9] |
| 403 | GACAGATGGAC | 0 | 2 | 4 | 0.6 | PF: GRAM KIAA1533 | Swissprot: |
| | | | | | | protein | tr\|Q8NC77] |
| 404 | AAGTGAGGAGA | 1 | 6 | 6 | 1.16 | PF: GRAM WW domain | Swissprot: |
| | | | | | | binding protein 2 | sp\|Q969T9] |
| 405 | TGCCGTGCCTG | 0 | 5 | 10 | 1.51 | PF: GRAM myotubularin | Swissprot: |
| | | | | | | related protein 1 | sp\|Q13613] |
| 406 | TAAAAGATGTA | 1 | 0 | −2 | 0.3 | PF: GRAM myotubularin | Swissprot: |
| | | | | | | related protein 2 | sp\|Q13614] |
| 407 | TTACACTGTAA | 0 | 1 | 2 | 0.3 | PF: GRAM neutral | Swissprot: |
| | | | | | | sphingomyelinase (N-SMase) | sp\|Q92636] |
| | | | | | | activation associated factor | |
| | | 30 | 13 | −2.31 | 2 | PF: GTP_CDC | 11 matches |
| 408 | ATTGTACAACA | 1 | 0 | −2 | 0.3 | PF: GTP_CDC CDC10 cell | Swissprot: |
| | | | | | | division cycle 10 homolog (S. | sp\|Q16181] |
| | | | | | | cerevisiae) | |
| 409 | GCCTCTTGAAG | 10 | 6 | −1.67 | 0.47 | PF: GTP_CDC CDC10 cell | Swissprot: |
| | | | | | | division cycle 10 homolog (S. | sp\|Q16181] |
| | | | | | | cerevisiae) | |
| 410 | GCCAACGGCGT | 1 | 0 | −2 | 0.3 | PF: GTP_CDC MLL septin- | Swissprot: |
| | | | | | | like fusion | tr\|Q96QF3] |
| 411 | TGGCCTGCCCA | 7 | 3 | −2.33 | 0.64 | PF: GTP_CDC MLL septin- | Swissprot: |
| | | | | | | like fusion | tr\|Q96QF3] |
| 412 | CTTGGTAATTT | 1 | 0 | −2 | 0.3 | PF: GTP_CDC hypothetical | Swissprot: |
| | | | | | | protein FLJ10849 | tr\|Q96KC0] |
| 413 | TTGCCTGCAGT | 0 | 1 | 2 | 0.3 | PF: GTP_CDC hypothetical | Swissprot: |
| | | | | | | protein FLJ10849 | tr\|Q96KC0] |
| 414 | AGTGTATCACA | 1 | 0 | −2 | 0.3 | PF: GTP_CDC hypothetical | Swissprot: |
| | | | | | | protein FLJ11619 | tr\|Q9H9P7] |
| 415 | CGGAGTCCATT | 7 | 1 | −7 | 1.4 | PF: GTP_CDC neural | Swissprot: |
| | | | | | | precursor cell expressed, | sp\|Q15019] |
| | | | | | | developmentally down- | |
| | | | | | | regulated 5 | |
| 416 | ATCCCTTCCCG | 1 | 0 | −2 | 0.3 | PF: GTP_CDC peanut-like 1 | Swissprot: |
| | | | | | | (Drosophila) | sp\|Q99719] |
| 417 | GGGCACAATGC | 1 | 0 | −2 | 0.3 | PF: GTP_CDC peanut-like 1 | Swissprot: |
| | | | | | | (Drosophila) | sp\|Q99719] |
| 418 | TGGCTGTTAAT | 0 | 2 | 4 | 0.6 | PF: GTP_CDC septin 6 | Swissprot: |
| | | | | | | | sp\|Q14141] |
| | | 3 | 20 | 6.67 | 3.57 | PF: PEPTIDASE_S9 | 9 matches |
| 419 | AGCTGATCAGC | 1 | 3 | 3 | 0.43 | PF: PEPTIDASE_S9 N- | Swissprot: |
| | | | | | | acylaminoacyl-peptide | sp\|P13798] |
| | | | | | | hydrolase | |
| 420 | CCATTTAAAGC | 0 | 1 | 2 | 0.3 | PF: PEPTIDASE_S9 | Swissprot: |
| | | | | | | dipeptidylpeptidase 4 (CD26, | sp\|P27487] |
| | | | | | | adenosine deaminase | |
| | | | | | | complexing protein 2) | |
| 421 | GCTGGGAACCC | 0 | 1 | 2 | 0.3 | PF: PEPTIDASE_S9 | Swissprot: |
| | | | | | | dipeptidylpeptidase 4 (CD26, | sp\|P27487] |
| | | | | | | adenosine deaminase | |
| | | | | | | complexing protein 2) | |
| 422 | CTCAAAATCAA | 0 | 1 | 2 | 0.3 | PF: PEPTIDASE_S9 | Swissprot: |
| | | | | | | dipeptidylpeptidase 8 | tr\|Q8IWG7] |
| 423 | GGGAAACCCCG | 0 | 7 | 14 | 2.11 | PF: PEPTIDASE_S9 | Swissprot: |
| | | | | | | dipeptidylpeptidase 9 | tr\|Q8N2J7] |
| 424 | GGGGAAACCCC | 0 | 1 | 2 | 0.3 | PF: PEPTIDASE_S9 | Swissprot: |
| | | | | | | dipeptidylpeptidase 9 | tr\|Q8N2J7] |
| 425 | TGTCTGCCTGA | 0 | 1 | 2 | 0.3 | PF: PEPTIDASE_S9 | Swissprot: |
| | | | | | | dipeptidylpeptidase 9 | tr\|Q8N2J7] |
| 426 | GAGAAGACTTC | 1 | 3 | 3 | 0.43 | PF: PEPTIDASE_S9 prolyl | Swissprot: |
| | | | | | | endopeptidase | sp\|P48147] |
| 427 | ATTTTTGGTGG | 1 | 2 | 2 | 0.21 | PF: PEPTIDASE_S9 putative L- | Swissprot: |
| | | | | | | type neutral amino acid | tr\|O43163] |
| | | | | | | transporter | |
| | | 200 | 260 | 1.3 | 2.35 | PF: RIBOSOMAL_S4E | 6 matches |
| 428 | ACTCTTAATGT | 0 | 2 | 4 | 0.6 | PF: RIBOSOMAL_S4E | Swissprot: |
| | | | | | | ribosomal protein S4, X-linked | sp\|P12750] |
| 429 | ATGCCCGCACC | 2 | 1 | −2 | 0.2 | PF: RIBOSOMAL_S4E | Swissprot: |
| | | | | | | ribosomal protein S4, X-linked | sp\|P12750] |
| 430 | GACAGGTAAAG | 1 | 0 | −2 | 0.3 | PF: RIBOSOMAL_S4E | Swissprot: |
| | | | | | | ribosomal protein S4, X-linked | sp\|P12750] |
| 431 | GATTTTTTTTC | 0 | 1 | 2 | 0.3 | PF: RIBOSOMAL_S4E | Swissprot: |
| | | | | | | ribosomal protein S4, X-linked | sp\|P12750] |
| 432 | TCAGATCTTTG | 196 | 255 | 1.3 | 2.32 | PF: RIBOSOMAL_S4E | Swissprot: |
| | | | | | | ribosomal protein S4, X-linked | sp\|P12750] |
| 433 | TCAGATTTTTG | 1 | 1 | 1 | 0 | PF: RIBOSOMAL_S4E | Swissprot: |
| | | | | | | ribosomal protein S4, X-linked | sp\|P12750] |

TABLE 9

| | | | | | | | |
|---|---|---|---|---|---|---|---|
| | Ana- | Kata- | | Signifi- | | | |
| Tags | gen | gen | Quot. | cance | Word | Description | Swiss-prot |

| | | | | | | | | |
|---|---|---|---|---|---|---|---|---|
| | | 1 | 16 | 16 | 3.85 | aciduria | 3 matches | |
| 434 | GAGAGCTACAT | 1 | 5 | 5 | 0.91 | aciduria | electron-transfer- | Swissprot: sp\| |
| | | | | | | | flavoprotein, alpha | P13804 |
| | | | | | | | polypeptide (glutaric | |
| | | | | | | | aciduria II) | |
| 435 | GCGATGGCCGT | 0 | 10 | 20 | 3.02 | aciduria | methylmalonic aciduria | Swissprot: tr\| |
| | | | | | | | (cobalamin deficiency) | Q96EY8 |
| | | | | | | | type B | |
| 436 | GTCTGCCCTCT | 0 | 1 | 2 | 0.3 | aciduria | mevalonate kinase | Swissprot: sp\| |
| | | | | | | | (mevalonic aciduria) | Q03426 |
| | | 19 | 5 | −3.8 | 2.38 | angiopoletin | 3 matches | |
| 437 | GTGCTGGTGCT | 1 | 1 | 1 | 0 | angiopoietin | angiopoietin-like 4 | Swissprot: sp\| |
| | | | | | | | | Q9BY76 |
| 438 | GATGCCGGCAC | 17 | 4 | −4.25 | 2.35 | angiopoietin | angiopoietin-like factor | Swissprot: tr\| |
| | | | | | | | | O43827 |
| 439 | CTCATTCGGCC | 1 | 0 | −2 | 0.3 | angiopoietin | angiopoietin-related | Swissprot: tr\| |
| | | | | | | | protein 5 | Q8N199 |
| | | 2 | 12 | 6.03 | 2.14 | autophagy | 4 matches | |
| 440 | GAGATTGAGGG | 0 | 2 | 4.02 | 0.6 | autophagy | APG10 autophagy 10- | Swissprot: tr\| |
| | | | | | | | like (S. cerevisiae) | Q9H0Y0 |
| 441 | AAAGTGGAAAC | 0 | 1 | 2.01 | 0.3 | autophagy | APG5 autophagy 5-like | Swissprot: sp\| |
| | | | | | | | (S. cerevisiae) | Q9H1Y0 |
| 442 | CTGAGGTGATG | 0 | 2 | 4.02 | 0.6 | autophagy | autophagy | Swissprot: tr\| |
| | | | | | | | Apg3p/Aut1p-like | Q9H6L9 |
| 443 | TCGGGTGTGGG | 2 | 7.01 | 3.51 | 0.97 | autophagy | cysteine protease | Swissprot: tr\| |
| | | | | | | | involved in autophagy | Q969K0 |
| | | | | | | | APG4-D | |
| | | 6 | 21 | 3.5 | 2.44 | camp | 11 matches | |
| 444 | CAATGTCTTCA | 0 | 1 | 2 | 0.3 | camp | Homo sapiens cDNA | |
| | | | | | | | FLJ33024 fis, clone | |
| | | | | | | | THYMU1000532, | |
| | | | | | | | moderately similar to | |
| | | | | | | | HIGH-AFFINITY CAMP- | |
| | | | | | | | SPECI . . . | |
| 445 | CCTCAGGCTCC | 0 | 2 | 4 | 0.6 | camp | cAMP responsive | Swissprot: tr\| |
| | | | | | | | element binding protein | O14671 |
| | | | | | | | 3 (luman) | |
| 446 | GACACCAGGGT | 2 | 5 | 2.5 | 0.54 | camp | cAMP responsive | Swissprot: sp\| |
| | | | | | | | element binding protein- | P22105 |
| | | | | | | | like 1 | |
| 447 | TTAATAAATGT | 1 | 1 | 1 | 0 | camp | cAMP responsive | Swissprot: tr\| |
| | | | | | | | element binding protein- | O60519 |
| | | | | | | | like 2 | |
| 448 | TTGGTTGCACT | 0 | 1 | 2 | 0.3 | camp | cAMP responsive | Swissprot: sp\| |
| | | | | | | | element modulator | Q03060 |
| 449 | CCCCGGGCCTC | 1 | 0 | −2 | 0.3 | camp | phosphodiesterase 4A, | |
| | | | | | | | cAMP-specific | |
| | | | | | | | (phosphodiesterase E2 | |
| | | | | | | | dunce homolog, | |
| | | | | | | | Drosophila) | |
| 450 | GAGCTCCACAG | 0 | 2 | 4 | 0.6 | camp | protein kinase (cAMP- | Swissprot: |
| | | | | | | | dependent, catalytic) | sp\|Q9Y2B9 |
| | | | | | | | inhibitor gamma | |
| 451 | TCCCCCCATTC | 0 | 1 | 2 | 0.3 | camp | protein kinase, cAMP- | Swissprot: |
| | | | | | | | dependent, catalytic, | sp\|P17612 |
| | | | | | | | alpha | |
| 452 | TTCAGTGGGTT | 1 | 1 | 1 | 0 | camp | protein kinase, cAMP- | Swissprot: |
| | | | | | | | dependent, catalytic, | sp\|P17612 |
| | | | | | | | alpha | |
| 453 | ACCAATTTAAA | 0 | 1 | 2 | 0.3 | camp | protein kinase, cAMP- | |
| | | | | | | | dependent, regulatory, | |
| | | | | | | | type I, alpha (tissue | |
| | | | | | | | specific extinguisher 1) | |
| 454 | TGTGCTAATAT | 1 | 6 | 6 | 1.16 | camp | protein kinase, cAMP- | |
| | | | | | | | dependent, regulatory, | |
| | | | | | | | type I, alpha (tissue | |
| | | | | | | | specific extinguisher 1) | |
| | | 27 | 7 | −3.86 | 3.27 | desmocollin | 4 matches | |
| 455 | GCATAGTTCTA | 2 | 0 | −4 | 0.6 | desmocollin | (Manual) DSC2 | Swissprot: sp\| |
| | | | | | | | Desmocollin-2A/2B | Q02487 |
| | | | | | | | (reverse tag) | |
| 456 | AGAGTCATACA | 5 | 0 | −10 | 1.5 | desmocollin | (Manual) DSC2 | Swissprot: sp\| |
| | | | | | | | Desmocollin-2A/2B | Q02487 |
| 457 | CAGGAGTGTGC | 17 | 5 | −3.4 | 1.96 | desmocollin | desmocollin 3 | Swissprot: sp\| |
| | | | | | | | | Q14574 |
| 458 | TATGCCCGAAT | 3 | 2 | −1.5 | 0.16 | desmocollin | desmocollin 3 | Swissprot: sp\| |
| | | | | | | | | Q14574 |
| | | 7 | 0 | −14 | 2.1 | dsc2 | 2 matches | |
| 459 | GCATAGTTCTA | 2 | 0 | −4 | 0.6 | dsc2 | (Manual) DSC2 | Swissprot: sp\| |
| | | | | | | | Desmocollin-2A/2B | Q02487 |
| | | | | | | | (reverse tag) | |
| 460 | AGAGTCATACA | 5 | 0 | −10 | 1.5 | dsc2 | (Manual) DSC2 | Swissprot: sp\| |
| | | | | | | | Desmocollin-2A/2B | Q02487 |
| | | 46 | 20 | −2.3 | 2.86 | gelsolin | 3 matches | |
| 461 | CTCCCCTGCCC | 8 | 5 | −1.6 | 0.37 | gelsolin | capping protein (actin | Swissprot: sp\| |
| | | | | | | | filament), gelsolin-like | P40121 |
| 462 | TTCCCCTGCCC | 1 | 0 | −2 | 0.3 | gelsolin | capping protein (actin | Swissprot: sp\| |
| | | | | | | | filament), gelsolin-like | P40121 |
| 463 | TCACCGGTCAG | 37 | 15 | −2.47 | 2.64 | gelsolin | gelsolin (amyloidosis, | Swissprot: sp\| |
| | | | | | | | Finnish type) | P06396 |
| | | 10 | 1 | −10 | 2.19 | gla | 2 matches | |
| 464 | TTCTCTCCACA | 1 | 0 | −2 | 0.3 | gla | bone gamma- | Swissprot: |
| | | | | | | | carboxyglutamate (gla) | sp\|P02818 |
| | | | | | | | protein (osteocalcin) | |
| 465 | GTTTATGGATA | 9 | 1 | −9 | 1.92 | gla | matrix Gla protein | Swissprot: sp\| |
| | | | | | | | | P08493 |
| | | 111 | 64 | −1.73 | 3.39 | lysosomal | 38 matches | |
| 466 | CAGTAAAAAAA | 1 | 0 | −2 | 0.3 | lysosomal | ATPase, H+ | Swissprot: sp\| |
| | | | | | | | transporting, lysosomal | O75348 |
| | | | | | | | 13 kDa, V1 subunit G | |
| | | | | | | | isoform 1 | |
| 467 | CATTTTTCCCC | 0 | 1 | 2 | 0.3 | lysosomal | ATPase, H+ | Swissprot: sp\| |
| | | | | | | | transporting, lysosomal | O75348 |
| | | | | | | | 13 kDa, V1 subunit G | |
| | | | | | | | isoform 1 | |
| 468 | TAACAAGTTCT | 1 | 0 | −2 | 0.3 | lysosomal | ATPase, H+ | Swissprot: sp\| |
| | | | | | | | transporting, lysosomal | O75348 |
| | | | | | | | 13 kDa, V1 subunit G | |
| | | | | | | | isoform 1 | |
| 469 | TATATCAGTGT | 1 | 1 | 1 | 0 | lysosomal | ATPase, H+ | Swissprot: sp\| |
| | | | | | | | transporting, lysosomal | O75348 |
| | | | | | | | 13 kDa, V1 subunit G | |
| | | | | | | | isoform 1 | |
| 470 | TATTACTTGGT | 1 | 0 | −2 | 0.3 | lysosomal | ATPase, H+ | Swissprot: sp\| |
| | | | | | | | transporting, lysosomal | O75348 |
| | | | | | | | 13 kDa, V1 subunit G | |
| | | | | | | | isoform 1 | |
| 471 | TTCACTGCCGA | 1 | 1 | 1 | 0 | lysosomal | ATPase, H+ | Swissprot: sp\| |
| | | | | | | | transporting, lysosomal | Q16864 |
| | | | | | | | 14 kDa, V1 subunit F | |
| 472 | CGCAGTGTCCT | 10 | 4 | −2.5 | 0.92 | lysosomal | ATPase, H+ | Swissprot: sp\| |
| | | | | | | | transporting, lysosomal | P27449 |
| | | | | | | | 16 kDa, V0 subunit c | |
| 473 | TTTGGGGCTGG | 12 | 4 | −3 | 1.3 | lysosomal | ATPase, H+ | Swissprot: sp\| |
| | | | | | | | transporting, lysosomal | Q99437 |
| | | | | | | | 21 kDa, V0 subunit c″ | |
| 474 | AATATGCTTTA | 3 | 3 | 1 | 0 | lysosomal | ATPase, H+ | Swissprot: sp\| |
| | | | | | | | transporting, lysosomal | P36543 |
| | | | | | | | 31 kDa, V1 subunit E | |
| | | | | | | | isoform 1 | |
| 475 | GGAGCCATTCT | 3 | 1 | −3 | 0.42 | lysosomal | ATPase, H+ | Swissprot: sp\| |
| | | | | | | | transporting, lysosomal | Q9Y5K8 |
| | | | | | | | 34 kDa, V1 subunit D | |
| 476 | GGAAGGACAGA | 7 | 3 | −2.33 | 0.64 | lysosomal | ATPase, H+ | Swissprot: sp\| |
| | | | | | | | transporting, lysosomal | P12953 |
| | | | | | | | 38 kDa, V0 subunit d | |
| | | | | | | | isoform 1 | |
| 477 | AAATACAGCAG | 3 | 4 | 1.33 | 0.14 | lysosomal | ATPase, H+ | Swissprot: tr\| |
| | | | | | | | transporting, lysosomal | Q8NEY4 |
| | | | | | | | 42 kDa, V1 subunit C | |
| | | | | | | | isoform 2 | |
| 478 | GCCGCCATCAA | 3 | 1 | −3 | 0.42 | lysosomal | ATPase, H+ | Swissprot: tr\| |
| | | | | | | | transporting, lysosomal | Q8NEY4 |
| | | | | | | | 42 kDa,V1 subunit C | |
| | | | | | | | isoform 2 | |
| 479 | TTTGCCTGTTA | 0 | 1 | 2 | 0.3 | lysosomal | ATPase, H+ | Swissprot: sp\| |
| | | | | | | | transporting, lysosomal | Q9UI12 |
| | | | | | | | 50/57 kDa, V1 subunit H | |
| 480 | TTTTTACAGTG | 1 | 0 | −2 | 0.3 | lysosomal | ATPase, H+ | Swissprot: sp\| |
| | | | | | | | transporting, lysosomal | P38606 |
| | | | | | | | 70 kDa, V1 subunit A, | |
| | | | | | | | isoform 1 | |
| 481 | CTCTACAGTGC | 1 | 1 | 1 | 0 | lysosomal | ATPase, H+ | Swissprot: sp\| |
| | | | | | | | transporting, lysosomal | O15342 |
| | | | | | | | 9 kDa, V0 subunit e | |
| 482 | TGGCTGTGAGG | 3 | 3 | 1 | 0 | lysosomal | ATPase, H+ | Swissprot: sp\| |
| | | | | | | | transporting, lysosomal | Q93050 |
| | | | | | | | V0 subunit a isoform 1 | |
| 483 | GGGTGCTTGGT | 4 | 4 | 1 | 0 | lysosomal | ATPase, H+ | Swissprot: sp\| |
| | | | | | | | transporting, lysosomal | Q15904 |
| | | | | | | | interacting protein 1 | |
| 484 | AATGTGATTTC | 0 | 1 | 2 | 0.3 | lysosomal | Homo sapiens cDNA | Homo |
| | | | | | | | FLJ33528 fis, clone | sapiens |
| | | | | | | | BRAMY2007110, highly | cDNA |
| | | | | | | | similar to LYSOSOMAL | FLJ33528 |
| | | | | | | | PRO-X | fis, clone |
| | | | | | | | CARBOXYPEPTI . . . | BRAMY2007 |
| | | | | | | | | 110, highly |
| | | | | | | | | similar to |
| | | | | | | | | LYSOSOMA |
| | | | | | | | | L PRO-X |
| | | | | | | | | CARBOXYP |
| | | | | | | | | EPTI . . . |
| 485 | GCGGTTGTGGC | 3 | 2 | −1.5 | 0.16 | lysosomal | Lysosomal-associated | Swissprot: sp\| |
| | | | | | | | multispanning | Q13571 |
| | | | | | | | membrane protein-5 | |
| 486 | CACCAGGCCAT | 1 | 0 | −2 | 0.3 | lysosomal | T-cell, immune regulator | |
| | | | | | | | 1, ATPase, H+ | |
| | | | | | | | transporting, lysosomal | |
| | | | | | | | V0 protein a isoform 3 | |
| 487 | GTGATGCGCAT | 1 | 1 | 1 | 0 | lysosomal | T-cell, immune regulator | |
| | | | | | | | 1, ATPase, H+ | |
| | | | | | | | transporting, lysosomal | |
| | | | | | | | V0 protein a isoform 3 | |
| 488 | CAGGTTGTGAG | 2 | 0 | −4 | 0.6 | lysosomal | acid phosphatase 2, | Swissprot: sp\| |
| | | | | | | | lysosomal | P11117 |
| 489 | GAAATACAGTT | 15 | 11 | −1.36 | 0.35 | lysosomal | cathepsin D (lysosomal | Swissprot: sp\| |
| | | | | | | | aspartyl protease) | P07339 |
| 490 | AGCTGAGCTAA | 4 | 2 | −2 | 0.34 | lysosomal | deoxyribonuclease II, | Swissprot: sp\| |
| | | | | | | | lysosomal | O00115 |
| 491 | AGAAGTGTCCT | 3 | 0 | −6 | 0.9 | lysosomal | lipase A, lysosomal acid, | Swissprot: sp\| |
| | | | | | | | cholesterol esterase | P38571 |
| | | | | | | | (Wolman disease) | |
| 492 | GGGCTCTGAGC | 1 | 1 | 1 | 0 | lysosomal | lysophospholipase 3 | Swissprot: tr\| |
| | | | | | | | (lysosomal | Q8NCC3 |
| | | | | | | | phospholipase A2) | |
| 493 | TCACTTGCTGT | 0 | 1 | 2 | 0.3 | lysosomal | lysosomal apyrase-like 1 | Swissprot: sp\| |
| | | | | | | | | Q9Y227 |
| 494 | ATAATTTTTAA | 1 | 0 | −2 | 0.3 | lysosomal | lysosomal-associated | Swissprot: sp\| |
| | | | | | | | membrane protein 1 | P11279 |
| 495 | CTCACACATTA | 7 | 3 | −2.33 | 0.64 | lysosomal | lysosomal-associated | Swissprot: sp\| |
| | | | | | | | membrane protein 1 | P11279 |
| 496 | CAAATAACAAG | 2 | 0 | −4 | 0.6 | lysosomal | lysosomal-associated | Swissprot: sp\| |
| | | | | | | | membrane protein 2 | P13473 |
| 497 | CAACTGCCTAT | 2 | 0 | −4 | 0.6 | lysosomal | lysosomal-associated | Swissprot: sp\| |
| | | | | | | | membrane protein 2 | P13473 |
| 498 | GCCATTATAAG | 2 | 0 | −4 | 0.6 | lysosomal | lysosomal-associated | Swissprot: sp\| |
| | | | | | | | membrane protein 2 | P13473 |
| 499 | TTTTTTCTTCA | 0 | 1 | 2 | 0.3 | lysosomal | lysosomal-associated | Swissprot: sp\| |
| | | | | | | | membrane protein 2 | P13473 |
| 500 | CAACCATCATC | 4 | 0 | −8 | 1.2 | lysosomal | lysosomal-associated | Swissprot: sp\| |
| | | | | | | | protein transmembrane | Q15012 |
| | | | | | | | 4 alpha | |
| 501 | TTTCTAGTTTG | 5 | 6 | 1.2 | 0.11 | lysosomal | lysosomal-associated | Swissprot: sp\| |
| | | | | | | | protein transmembrane | Q15012 |
| | | | | | | | 4 alpha | |
| 502 | ACTGACTATCA | 1 | 1 | 1 | 0 | lysosomal | sialidase 1 (lysosomal | Swissprot: sp\| |
| | | | | | | | sialidase) | Q99519 |
| 503 | GAGTAGAGGCC | 2 | 2 | 1 | 0 | lysosomal | sphingomyelin | Swissprot: sp\| |
| | | | | | | | phosphodiesterase 1, | P17405 |
| | | | | | | | acid lysosomal (acid | |
| | | | | | | | sphingomyelinase) | |
| | | 91 | 59 | −1.54 | 2.01 | monooxy- | 16 matches | |
| | | | | | | genase | | |
| 504 | ACGACAAAGCT | 0 | 1 | 2 | 0.3 | monooxy- | peptidylglycine alpha- | Swissprot: sp\| |
| | | | | | | genase | amidating | P19021 |
| | | | | | | | monooxygenase | |
| 505 | CAGTTACTTAG | 3 | 3 | 1 | 0 | monooxy- | tyrosine 3- | |
| | | | | | | genase | monooxygenase/tryptop | |
| | | | | | | | han 5-monooxygenase | |
| | | | | | | | activation protein, beta | |
| | | | | | | | polypeptide | |
| 506 | CTTTTCAGCAA | 3 | 2 | −1.5 | 0.16 | monooxy- | tyrosine 3- | Swissprot: |
| | | | | | | genase | monooxygenase/tryptop | SWALL: |
| | | | | | | | han 5-monooxygenase | AAP35825 |
| | | | | | | | activation protein, | |
| | | | | | | | epsilon polypeptide | |
| 507 | GAATTAACATT | 3 | 4 | 1.33 | 0.14 | monooxy- | tyrosine 3- | Swissprot: |
| | | | | | | genase | monooxygenase/tryptop | SWALL: |
| | | | | | | | han 5-monooxygenase | AAP35825 |
| | | | | | | | activation protein, | |
| | | | | | | | epsilon polypeptide | |
| 508 | GCGCTGTCAGG | 3 | 1 | −3 | 0.42 | monooxy- | tyrosine 3- | |
| | | | | | | genase | monooxygenase/tryptop | |
| | | | | | | | han 5-monooxygenase | |
| | | | | | | | activation protein, eta | |
| | | | | | | | polypeptide | |
| 509 | TCAATCAAGAT | 1 | 2 | 2 | 0.21 | monooxy- | tyrosine 3- | |
| | | | | | | genase | monooxygenase/tryptop | |
| | | | | | | | han 5-monooxygenase | |
| | | | | | | | activation protein, eta | |
| | | | | | | | polypeptide | |
| 510 | AATGTGAGTCA | 5 | 7 | 1.4 | 0.24 | monooxy- | tyrosine 3- | |
| | | | | | | genase | monooxygenase/tryptop | |
| | | | | | | | han 5-monooxygenase | |
| | | | | | | | activation protein, | |
| | | | | | | | gamma polypeptide | |
| 511 | TCACTATAGCA | 1 | 0 | −2 | 0.3 | monooxy- | tyrosine 3- | |
| | | | | | | genase | monooxygenase/tryptop | |
| | | | | | | | han 5-monooxygenase | |
| | | | | | | | activation protein, | |
| | | | | | | | gamma polypeptide | |
| 512 | CTCTTAATGTA | 1 | 0 | −2 | 0.3 | monooxy- | tyrosine 3- | |
| | | | | | | genase | monooxygenase/tryptop | |
| | | | | | | | han 5-monooxygenase | |
| | | | | | | | activation protein, theta | |
| | | | | | | | polypeptide | |
| 513 | GGCCATCTCTT | 30 | 17 | −1.76 | 1.21 | monooxy- | tyrosine 3- | |
| | | | | | | genase | monooxygenase/tryptop | |
| | | | | | | | han 5-monooxygenase | |
| | | | | | | | activation protein, theta | |
| | | | | | | | polypeptide | |
| 514 | TGAAAGGGTGT | 1 | 0 | −2 | 0.3 | monooxy- | tyrosine 3- | |
| | | | | | | genase | monooxygenase/tryptop | |
| | | | | | | | han 5-monooxygenase | |
| | | | | | | | activation protein, theta | |
| | | | | | | | polypeptide | |
| 515 | TGAGAGGGTGT | 25 | 10 | −2.5 | 1.93 | monooxy- | tyrosine 3- | |
| | | | | | | genase | monooxygenase/tryptop | |
| | | | | | | | han 5-monooxygenase | |
| | | | | | | | activation protein, theta | |
| | | | | | | | polypeptide | |
| 516 | ATCTTTCTGGC | 10 | 5 | −2 | 0.67 | monooxy- | tyrosine 3- | Swissprot: |
| | | | | | | genase | monooxygenase/tryptop | SWALL: |
| | | | | | | | han 5-monooxygenase | AAH50891 |
| | | | | | | | activation protein, zeta | |
| | | | | | | | polypeptide | |
| 517 | GCCACCAAGTA | 2 | 0 | −4 | 0.6 | monooxy- | tyrosine 3- | Swissprot: |
| | | | | | | genase | monooxygenase/tryptop | SWALL: |
| | | | | | | | han 5-monooxygenase | AAH50891 |
| | | | | | | | activation protein, zeta | |
| | | | | | | | polypeptide | |
| 518 | TAAGTGGAATA | 2 | 6 | 3 | 0.75 | monooxy- | tyrosine 3- | Swissprot: |
| | | | | | | genase | monooxygenase/tryptop | SWALL: |
| | | | | | | | han 5-monooxygenase | AAH50891 |
| | | | | | | | activation protein, zeta | |
| | | | | | | | polypeptide | |
| 519 | TTAGGCAAGTA | 1 | 1 | 1 | 0 | monooxy- | tyrosine 3- | Swissprot: |
| | | | | | | genase | monooxygenase/tryptop | SWALL: |
| | | | | | | | han 5-monooxygenase | AAH50891 |
| | | | | | | | activation protein, zeta | |
| | | | | | | | polypeptide | |
| | | 755 | 153 | −4.93 | 95.23 | rrna | 22 matches | |
| 520 | AATGGATGAAC | 2 | 0 | −4 | 0.6 | rrna | rRNA intermediate tag | Swissprot: |
| | | | | | | | | none |
| 521 | ATTAAGAGGGA | 5 | 2 | −2.5 | 0.53 | rrna | rRNA intermediate tag | Swissprot: |
| | | | | | | | | none |
| 522 | CCAGAGGCTGT | 17 | 4 | −4.25 | 2.35 | rrna | rRNA intermediate tag | Swissprot: |
| | | | | | | | | none |
| 523 | CCGACGGGCGC | 15 | 1 | −15 | 3.55 | rrna | rRNA intermediate tag | Swissprot: |
| | | | | | | | | none |
| 524 | CGCGTCACTAA | 8 | 0 | −16 | 2.4 | rrna | rRNA intermediate tag | Swissprot: |
| | | | | | | | | none |
| 525 | CTAACTAGTTA | 2 | 0 | −4 | 0.6 | rrna | rRNA intermediate tag | Swissprot: |
| | | | | | | | | none |
| 526 | GCAACAACACA | 19 | 4 | −4.75 | 2.8 | rrna | rRNA intermediate tag | Swissprot: |
| | | | | | | | | none |
| 527 | GCCGTTCTTAG | 46 | 8 | −5.75 | 7.06 | rrna | rRNA intermediate tag | Swissprot: |
| | | | | | | | | none |
| 528 | CCTGTCATCCC | 2 | 2 | 1 | 0 | rrna | rRNA intermediate tag, | Swissprot: |
| | | | | | | | Alu | none |
| 529 | GAACCCTTCTC | 2 | 0 | −4 | 0.6 | rrna | rRNA intermediate tag, | Swissprot: |
| | | | | | | | Alu | none |
| 530 | ACCCGCCGGGC | 26 | 11 | −2.36 | 1.84 | rrna | rRNA major tag | Swissprot: |
| | | | | | | | | none |
| 531 | AGAGGTGTAGA | 19 | 2 | −9.5 | 3.9 | rrna | rRNA major tag | Swissprot: |
| | | | | | | | | none |
| 532 | GAAGTCGGAAT | 11 | 4 | −2.75 | 1.11 | rrna | rRNA major tag | Swissprot: |
| | | | | | | | | none |
| 533 | GGTCAGTCGGT | 14 | 3 | −4.67 | 2.11 | rrna | rRNA major tag | Swissprot: |
| | | | | | | | | none |
| 534 | GTAATCCTGCT | 24 | 8 | −3 | 2.33 | rrna | rRNA major tag | Swissprot: |
| | | | | | | | | none |
| 535 | GTGACCACGGG | 493 | 68 | −7.25 | 79.68 | rrna | rRNA major tag | Swissprot: |
| | | | | | | | | none |
| 536 | TGGCGTACGGA | 4 | 3 | −1.33 | 0.14 | rrna | rRNA major tag | Swissprot: |
| | | | | | | | | none |
| 537 | TTGGAACAATG | 3 | 1 | −3 | 0.42 | rrna | rRNA major tag | Swissprot: |
| | | | | | | | | none |
| 538 | AGCCACCGCGC | 1 | 2 | 2 | 0.21 | rrna | rRNA major tag, Alu | Swissprot: |
| | | | | | | | | none |
| 539 | CCTATAATCCC | 5 | 5 | 1 | 0 | rrna | rRNA major tag, Alu | Swissprot: |
| | | | | | | | | none |
| 540 | TTGGTCAGGCT | 33 | 24 | −1.38 | 0.61 | rrna | rRNA major tag, Alu | Swissprot: |
| | | | | | | | | none |
| 541 | GTAGGCACGGC | 4 | 1 | −4 | 0.66 | rrna | rRNA minor tag | Swissprot: |
| | | | | | | | | none |
| | | 46 | 20 | −2.3 | 2.86 | seleno- | 14 matches | |
| | | | | | | protein | | |
| 542 | TAAGCCCTTTT | 1 | 0 | −2 | 0.3 | seleno- | 15 kDa selenoprotein | Swiss-prot: |
| | | | | | | protein | | sp\|O60613 |
| 543 | TGCTGTGTGCT | 3 | 0 | −6 | 0.9 | seleno- | 15 kDa selenoprotein | Swiss- |
| | | | | | | protein | | prot: sp\|O606 |
| | | | | | | | | 13 |
| 544 | GGCAGAGGGCT | 5 | 2 | −2.5 | 0.53 | seleno- | elongation factor for | Swissprot: sp\| |
| | | | | | | protein | selenoprotein translation | P57772 |
| 545 | GTTTCTTCCCT | 5 | 0 | −10 | 1.5 | seleno- | selenoprotein H | Swissprot: tr\| |
| | | | | | | protein | | Q8IZQ5 |
| 546 | CAGTTCCATAA | 4 | 1 | −4 | 0.66 | seleno- | selenoprotein K | Swissprot: sp\| |
| | | | | | | protein | | Q9Y6D0 |
| 547 | CCCTGTAATAA | 4 | 4 | 1 | 0 | seleno- | selenoprotein N, 1 | Swissprot: sp\| |
| | | | | | | protein | | Q9NZV5 |
| 548 | AATAAAGCCTT | 6 | 2 | −3 | 0.74 | seleno- | selenoprotein P, | Swissprot: sp\| |
| | | | | | | protein | plasma, 1 | P49908 |
| 549 | GAGAAATCTAC | 0 | 1 | 2 | 0.3 | seleno- | selenoprotein P, | Swissprot: sp\| |
| | | | | | | protein | plasma, 1 | P49908 |
| 550 | TCTTTGTTGTT | 6 | 1 | −6 | 1.15 | seleno- | selenoprotein P, | Swissprot: sp\| |
| | | | | | | protein | plasma, 1 | P49908 |
| 551 | TGTGATAGTAA | 1 | 2 | 2 | 0.21 | seleno- | selenoprotein P, | Swissprot: sp\| |
| | | | | | | protein | plasma, 1 | P49908 |
| 552 | CCTTGACCAAT | 2 | 3 | 1.5 | 0.17 | seleno- | selenoprotein T | Swissprot: sp\| |
| | | | | | | protein | | Q9NZJ3 |
| 553 | GTGTGGTATTC | 2 | 0 | −4 | 0.6 | seleno- | selenoprotein T | Swissprot: sp\| |
| | | | | | | protein | | Q9NZJ3 |
| 554 | TCTTCCCCAGT | 4 | 2 | −2 | 0.34 | seleno- | selenoprotein W, 1 | Swissprot: sp\| |
| | | | | | | protein | | O15532 |
| 555 | CTCGGAGGCCT | 3 | 2 | −1.5 | 0.16 | seleno- | selenoprotein X, 1 | Swissprot: sp\| |
| | | | | | | protein | | Q9NZV6 |
| | | 91 | 58 | −1.57 | 2.13 | tryptophan | 15 matches | |
| 556 | CAGTTACTTAG | 3 | 3 | 1 | 0 | tryptophan | tyrosine 3- | |
| | | | | | | | monooxygenase/ | |
| | | | | | | | tryptophan 5- | |
| | | | | | | | monooxygenase | |
| | | | | | | | activation protein, beta | |
| | | | | | | | polypeptide | |
| 557 | CTTTTCAGCAA | 3 | 2 | −1.5 | 0.16 | tryptophan | tyrosine 3- | Swisaprot: |
| | | | | | | | monooxygenase/ | SWALL: |
| | | | | | | | tryptophan 5- | AAP35825 |
| | | | | | | | monooxygenase | |
| | | | | | | | activation protein, | |
| | | | | | | | epsilon polypeptide | |
| 558 | GAATTAACATT | 3 | 4 | 1.33 | 0.14 | tryptophan | tyrosine 3-monooxy- | Swissprot: |
| | | | | | | | genase/tryptophan 5- | SWALL: |
| | | | | | | | monooxygenase | AAP35825 |
| | | | | | | | activation protein, | |
| | | | | | | | epsilon polypeptide | |
| 559 | GCGCTGTCAGG | 3 | 1 | −3 | 0.42 | tryptophan | tyrosine 3-monooxy- | |
| | | | | | | | genase/tryptophan 5- | |
| | | | | | | | monooxygenase | |
| | | | | | | | activation protein, eta | |
| | | | | | | | polypeptide | |
| 560 | TCAATCAAGAT | 1 | 2 | 2 | 0.21 | tryptophan | tyrosine 3-monooxy- | |
| | | | | | | | genase/tryptophan 5- | |
| | | | | | | | monooxygenase | |
| | | | | | | | activation protein, eta | |
| | | | | | | | polypeptide | |
| 561 | AATGTGAGTCA | 5 | 7 | 1.4 | 0.24 | tryptophan | tyrosine 3-monooxy- | |
| | | | | | | | genase/tryptophan 5- | |
| | | | | | | | monooxygenase | |
| | | | | | | | activation protein, | |
| | | | | | | | gamma polypeptide | |
| 562 | TCACTATAGCA | 1 | 0 | −2 | 0.3 | tryptophan | tyrosine 3-monooxy- | |
| | | | | | | | genase/tryptophan 5- | |
| | | | | | | | monooxygenase | |
| | | | | | | | activation protein, | |
| | | | | | | | gamma polypeptide | |
| 563 | CTCTTAATGTA | 1 | 0 | −2 | 0.3 | tryptophan | tyrosine 3-monooxy- | |
| | | | | | | | genase/tryptophan 5- | |
| | | | | | | | monooxygenase | |
| | | | | | | | activation protein, theta | |
| | | | | | | | polypeptide | |
| 564 | GGCCATCTCTT | 30 | 17 | −1.76 | 1.21 | tryptophan | tyrosine 3-monooxy- | |
| | | | | | | | genase/tryptophan 5- | |
| | | | | | | | monooxygenase | |
| | | | | | | | activation protein, theta | |
| | | | | | | | polypeptide | |
| 565 | TGAAAGGGTGT | 1 | 0 | −2 | 0.3 | tryptophan | tyrosine 3-monooxy- | |
| | | | | | | | genase/tryptophan 5- | |
| | | | | | | | monooxygenase | |
| | | | | | | | activation protein, theta | |
| | | | | | | | polypeptide | |
| 566 | TGAGAGGGTGT | 25 | 10 | −2.5 | 1.93 | tryptophan | tyrosine 3-monooxy- | |
| | | | | | | | genase/tryptophan 5- | |
| | | | | | | | monooxygenase | |
| | | | | | | | activation protein, theta | |
| | | | | | | | polypeptide | |
| 567 | ATCTTTCTGGC | 10 | 5 | −2 | 0.67 | tryptophan | tyrosine 3-monooxy- | Swissprot: |
| | | | | | | | genase/tryptophan 5- | SWALL: |
| | | | | | | | monooxygenase | AAH50891 |
| | | | | | | | activation protein, zeta | |
| | | | | | | | polypeptide | |
| 568 | GCCACCAAGTA | 2 | 0 | −4 | 0.6 | tryptophan | tyrosine 3-monooxy- | Swissprot: |
| | | | | | | | genase/tryptophan 5- | SWALL: |
| | | | | | | | monooxygenase | AAH50891 |
| | | | | | | | activation protein, zeta | |
| | | | | | | | polypeptide | |
| 569 | TAAGTGGAATA | 2 | 6 | 3 | 0.75 | tryptophan | tyrosine 3-monooxy- | Swissprot: |
| | | | | | | | genase/tryptophan 5- | SWALL: |
| | | | | | | | monooxygenase | AAH50891 |
| | | | | | | | activation protein, zeta | |
| | | | | | | | polypeptide | |
| 570 | TTAGGCAAGTA | 1 | 1 | 1 | 0 | tryptophan | tyrosine 3-monooxy- | Swissprot: |
| | | | | | | | genase/tryptophan 5- | SWALL: |
| | | | | | | | monooxygenase | AAH50891 |
| | | | | | | | activation protein, zeta | |
| | | | | | | | polypeptide | |

1. An in vitro method for determining hair cycle phase in humans, comprising:
a) providing a plurality of genetically encoded markers isolated from hair covered human skin or from human hair follicles which are differentially expressed at an anagenic phase of the hair cycle when compared to expression in cells in the catagenic phase of the hair cycle;
b) obtaining a sample of hair covered skin or human hair follicles;
c) analyzing the sample of b) for the presence and optionally the quantity of at least one genetically encoded molecule which is differentially expressed in anagenic and catagenic hair follicles and
d) designating the sample as comprising healthy cells in the anagenic phase of the cycle if it contains markers which are expressed at higher levels in anagenic hair follicles or cells in regression in the catagenic phase if it contains molecules which are expressed at higher levels in catagenic hair follicles.

2. The method of claim 1 wherein said genetically encoded markers comprise at least one mRNA molecule, at least one protein or polypeptide or fragments thereof.

3. The method of claim 1, wherein said sample is assessed for the presence and optionally the quantity of a molecule selected from the group consisting of SEQ ID NO: 434 to SEQ ID NO: 570 or the corresponding gene product and the sample is designated as comprising healthy cells in the anagenic phase of the hair cycle if it contains molecules which are expressed more strongly in anagenic hair follicles than in catagenic hair follicles or cells in regression in the catagenic phase of the hair cycle if the sample contains molecules which are expressed more strongly in catagenic hair follicles than in anagenic hair follicles.

4. The method of claim 1 wherein said sample is assessed for the presence and optionally the quantity of a molecule selected from the group consisting of SEQ ID NO: 352 to SEQ ID NO: 433 or the corresponding gene product and the sample is designated as comprising healthy cells in the anagenic phase of the hair cycle if it contains molecules which are expressed more strongly in anagenic hair follicles than in catagenic hair follicles or cells in regression in the catagenic phase of the hair cycle if the sample contains molecules which are expressed more strongly in catagenic hair follicles than in anagenic hair follicles.

5. The method of claim 1, wherein said sample is assessed for the presence and optionally the quantity of a molecule selected from the group consisting of SEQ ID NO: 142 to SEQ ID NO: 351 or the corresponding gene product and the sample is designated as comprising healthy cells in the anagenic phase of the hair cycle if it contains molecules which are expressed more strongly in anagenic hair follicles than in catagenic hair follicles or cells in regression in the catagenic phase of the hair cycle if the sample contains molecules which are expressed more strongly in catagenic hair follicles than in anagenic hair follicles.

6. The method of claim 1, wherein said sample is assessed for the presence and optionally the quantity of a molecule selected from the group consisting of SEQ ID NO: 105 to SEQ ID NO: 141 or the corresponding gene product and the sample is designated as comprising healthy cells in the anagenic phase of the hair cycle if it contains molecules which are expressed at least twice as strongly in anagenic hair follicles when compared to expression in catagenic hair follicles or cells in regression in the catagenic phase of the hair cycle if it contains molecules which are expressed at least twice as strongly in catagenic hair follicles than in anagenic hair follicles.

7. The method of claim 1, wherein said sample is assessed for the presence and optionally the quantity of a molecule selected from the group consisting of SEQ ID NO: 43 to SEQ ID NO: 104 or the corresponding gene product and the sample is designated as comprising healthy cells in the anagenic phase of the hair cycle if it contains molecules which are expressed at least five times more strongly in anagenic hair follicles when compared to expression in catagenic hair follicles or cells in regression in the catagenic phase of the hair cycle if the sample contains molecules which are expressed at least five times more strongly in catagenic hair follicles than in anagenic hair follicles.

8. The method of claim 1, wherein said sample is assessed for the presence and optionally the quantity of a molecule selected from the group consisting of SEQ ID NO: 29 to SEQ ID NO: 42 or the corresponding gene product and the sample is designated as comprising healthy cells in the anagenic phase of the hair cycle if it contains molecules which are expressed at least 1.3 times more strongly in anagenic hair follicles when compared to expression in catagenic hair follicles or cells in regression in the catagenic phase of the hair cycle if the sample contains molecules which are expressed at least 1.3 times more strongly in catagenic hair follicles than in anagenic hair follicles.

9. The method of claim 1, wherein said sample is assessed for the presence and optionally the quantity of a molecule selected from the group consisting of SEQ ID NO: 13 to SEQ ID NO: 28 or the corresponding gene product and the sample is designated as healthy cells in the anagenic phase of the hair cycle if it contains molecules which are expressed at twice as strongly in anagenic hair follicles when compared to expression in catagenic hair follicles or cells in regression in the catagenic phase of the hair cycle if it contains molecules which are expressed at least twice as strongly in catagenic hair follicles than in anagenic hair follicles.

10. The method of claim 1, wherein said sample is assessed for the presence and optionally the quantity of a molecule selected from the group consisting of SEQ ID NO: 1 to SEQ ID NO: 12 or the corresponding gene product and the sample is designated as comprising healthy cells in the anagenic phase of the hair cycle if it contains molecules which are expressed at five times more strongly in anagenic hair follicles when compared to expression in catagenic hair follicles or cells in regression in the catagenic phase of the hair cycle if the sample contains molecules which are expressed at least five times more strongly in catagenic hair follicles than in anagenic hair follicles.

11. A method as claimed in claim 1, comprising
a) quantifying expression levels of at least two molecules in the sample which are differentially expressed in cells from the anagenic phase of the hair cycle when compared to expression levels in the catagenic phase of the hair cycle;
b) determining the expression ratios of said at least two molecules thereby forming an expression quotient; and
c) comparing the expression ratios obtained with those in column 5 of Tables 2 to 6 and designating the sample as comprising healthy cells in the anagenic phase of the hair cycle if the expression ratios observed in the follicles correspond to the ratios observed in anagenic hair follicles or cells in regression in the catagenic phase of the hair cycle if the expression ratios correspond to those observed in catagenic hair follicles.

12. A test kit for determining hair cycle phase in humans, said test kit comprising reagents suitable for performing the method of claim 1.

13. A test kit for determining hair cycle phase in humans, said test kit comprising reagents suitable for performing the method of claim 11.

14. A biochip for determining the hair cycle phase in human beings in vitro comprising a solid, i.e. rigid or flexible, carrier and a plurality of probes immobilized thereon which are capable of specifically binding to at least one molecule selected from the group consisting of SEQ ID NO: 1 to SEQ ID NO: 570 or the corresponding gene product.

15. A marker for determining hair cycle phase in human beings in vitro, selected from the group consisting of at least one molecule having a Swissprot Accession Number provided in column 8 of Table 8, a Swissprot Accession Number provided in column 9 of Table 7, a Swissprot Accession Number provided in column 9 of Table 9, a UniGene Accession Number provided in column 7 of Tables 2 to 6, and a Swissprot Accession Number in column 8 of Tables 2 to 6.

16. An in vitro method for identifying a pharmaceutically active agent which modulates the hair cycle, comprising
a) providing a sample hair covered human skin or human follicles comprising cells;
b) determining the phase of the hair cycle of said sample as claimed in claim 1;
c) contacting said cells with said agent at least once; and
d) repeating step b) to determine whether said agent alters the phase of the hair cycle.

17. The method of claim 16, wherein said cells are diseased and exhibit an impairment of hair growth.

18. The method of claim 16, wherein said agent stimulates cells to enter the anagen phase of hair growth.

19. The method of claim 16, performed on a biochip.

20. A test kit for identifying a pharmaceutically active agent which modulates the hair cycle, comprising means for carrying out the test method claimed in claim 16.

21. A marker for use in the method of claim 16, selected from the group consisting of at least one molecule or fragment thereof having a Swissprot Accession Number provided in column 8 of Table 8, a Swissprot Accession Number provided in column 9 of Table 7, A Swissprot Accession Number provided in column 9 of Table 9, a UniGene Accession Number in column 7 of Tables 2 to 6, and a Swissprot Accession Number in column 8 of Tables 2 to 6 or the corresponding gene product.

22. A pharmaceutical preparation comprising the agent identified in claim 16 having efficacy against diseases or impairment of hair and its growth in a pharmaceutically acceptable carrier.


SEQUENCE LISTING (570 sequences)

SEQ ID NO: 1 - DNA, 11 bp, Homo sapiens
gcgatggccgt

SEQ ID NO: 2 - DNA, 11 bp, Homo sapiens
aactcttgaag

SEQ ID NO: 3 - DNA, 31 bp, Homo sapiens
tgtctgcctgaggggaaccccgggaaccccg

SEQ ID NO: 4 - DNA, 11 bp, Homo sapiens
caacattcctg

SEQ ID NO: 5 - DNA, 11 bp, Homo sapiens
tcaatattctt

SEQ ID NO: 6 - DNA, 21 bp, Homo sapiens
gtgagttgggctggcagattg

SEQ ID NO: 7 - DNA, 22 bp, Homo sapiens
ttctaactccttaccagtgtac

SEQ ID NO: 8 - DNA, 22 bp, Homo sapiens
tgaatgagcactctctacagaa

SEQ ID NO: 9 - DNA, 11 bp, Homo sapiens
ttgctagaggg

SEQ ID NO: 10 - DNA, 22 bp, Homo sapiens
gcatagttctaagagtcataca

SEQ ID NO: 11 - DNA, 33 bp, Homo sapiens
ctccctctgcccccccaattctaaaactgggga

SEQ ID NO: 12 - DNA, 11 bp, Homo sapiens
accggcgcccg

SEQ ID NO: 13 - DNA, 22 bp, Homo sapiens
atcagtggcttaaggaatcggg

SEQ ID NO: 14 - DNA, 11 bp, Homo sapiens
actttttcaaa

SEQ ID NO: 15 - DNA, 55 bp, Homo sapiens
gggtaggggggctgtacttgtgcagcacggatgagattccagccaaaaacattcc

SEQ ID NO: 16 - DNA, 11 bp, Homo sapiens
taccctaaaac

SEQ ID NO: 17 - DNA, 11 bp, Homo sapiens
atttgagaagc

SEQ ID NO: 18 - DNA, 22 bp, Homo sapiens
tggaagcagatcggggtggccg

SEQ ID NO: 19 - DNA, 11 bp, Homo sapiens
aaagcacaagt

SEQ ID NO: 20 - DNA, 33 bp, Homo sapiens
gccggggtgttctagcctcacgctagccctcac

SEQ ID NO: 21 - DNA, 44 bp, Homo sapiens
tagggcaatcttaacagctacgctcattcagctccactaatgga

SEQ ID NO: 22 - DNA, 11 bp, Homo sapiens
tcaccggtcag

SEQ ID NO: 23 - DNA, 11 bp, Homo sapiens
gtaatcctgct

SEQ ID NO: 24 - DNA, 11 bp, Homo sapiens
gttccctggcc

SEQ ID NO: 25 - DNA, 11 bp, Homo sapiens
gatgccggcac

SEQ ID NO: 26 - DNA, 11 bp, Homo sapiens
ccagaggctgt

SEQ ID NO: 27 - DNA, 11 bp, Homo sapiens
ggtcagtcggt

SEQ ID NO: 28 - DNA, 11 bp, Homo sapiens
gcaacaacaca

SEQ ID NO: 29 - DNA, 11 bp, Homo sapiens
cctcaggatac

SEQ ID NO: 30 - DNA, 11 bp, Homo sapiens
tttcctctcaa

SEQ ID NO: 31 - DNA, 55 bp, Homo sapiens
tcaagccatcaggatatgtggtgatttcgttttctcacctctagcagttcattat

SEQ ID NO: 32 - DNA, 33 bp, Homo sapiens
tagaccccttgtaccatcaatagcctccaagga

SEQ ID NO: 33 - DNA, 11 bp, Homo sapiens
ttcatacacct

SEQ ID NO: 34 - DNA, 11 bp, Homo sapiens
tgatttcactt

SEQ ID NO: 35 - DNA, 11 bp, Homo sapiens
cactactcacc

SEQ ID NO: 36 - DNA, 11 bp, Homo sapiens
taaggagctga

SEQ ID NO: 37 - DNA, 66 bp, Homo sapiens
ttggcagcccagggtcctctccgggggagtttcgagggagtttcgagggaatttcatgaattaaaa

SEQ ID NO: 38 - DNA, 44 bp, Homo sapiens
tcagatttttgtcagatctttggtttgttgcccatgcccgcacc

SEQ ID NO: 39 - DNA, 44 bp, Homo sapiens
gtccgagtgcagggacgagtgacacatatatacatccctagtac

SEQ ID NO: 40 - DNA, 11 bp, Homo sapiens
gctggagttgc

SEQ ID NO: 41 - DNA, 11 bp, Homo sapiens
tcgaagccccc

SEQ ID NO: 42 - DNA, 55 bp, Homo sapiens
tgagagggtgttgaaagggtgtggccatctcttgaaaagtactactcttaatgta

SEQ ID NO: 43 - DNA, 22 bp, Homo sapiens
tgggcccgtgtataaaaagcag

SEQ ID NO: 44 - DNA, 11 bp, Homo sapiens
actcagaagag

SEQ ID NO: 45 - DNA, 11 bp, Homo sapiens
agggaggggcc

SEQ ID NO: 46 - DNA, 11 bp, Homo sapiens
cttttcttctg

SEQ ID NO: 47 - DNA, 22 bp, Homo sapiens
cctgtaaagccactcgtattag

SEQ ID NO: 48 - DNA, 11 bp, Homo sapiens
aaggcgtttcc

SEQ ID NO: 49 - DNA, 11 bp, Homo sapiens
cctgtgtgtgt

SEQ ID NO: 50 - DNA, 22 bp, Homo sapiens
cccaggagcagcagcaggagca

SEQ ID NO: 51 - DNA, 11 bp, Homo sapiens
acctgcccctc

SEQ ID NO: 52 - DNA, 11 bp, Homo sapiens
gtggggggagg

SEQ ID NO: 53 - DNA, 22 bp, Homo sapiens
tctgtgacttcagttttatttg

SEQ ID NO: 54 - DNA, 22 bp, Homo sapiens
gcctggtgaccagaagaatggg

SEQ ID NO: 55 - DNA, 33 bp, Homo sapiens
tgcaagaagtactttagctacccttacgtgatt

SEQ ID NO: 56 - DNA, 22 bp, Homo sapiens
gttatatgcccggttttagttc

SEQ ID NO: 57 - DNA, 11 bp, Homo sapiens
ttacaacattg

SEQ ID NO: 58 - DNA, 33 bp, Homo sapiens
ttttaaacttgtctccatcactgcttgaactct

SEQ ID NO: 59 - DNA, 22 bp, Homo sapiens
gctgtatgtacgcaaggttggt

SEQ ID NO: 60 - DNA, 22 bp, Homo sapiens
tggacaggcagctttccccttt

SEQ ID NO: 61 - DNA, 11 bp, Homo sapiens
acatcatactg

SEQ ID NO: 62 - DNA, 11 bp, Homo sapiens
atgcaagagag

SEQ ID NO: 63 - DNA, 11 bp, Homo sapiens
ccaagaaagaa

SEQ ID NO: 64 - DNA, 11 bp, Homo sapiens
gatttgtgttc

SEQ ID NO: 65 - DNA, 11 bp, Homo sapiens
gcgagaatcca

SEQ ID NO: 66 - DNA, 11 bp, Homo sapiens
ggccagcaagt

SEQ ID NO: 67 - DNA, 11 bp, Homo sapiens
ggtgacagaga

SEQ ID NO: 68 - DNA, 11 bp, Homo sapiens
tgtaaagattt

SEQ ID NO: 69 - DNA, 11 bp, Homo sapiens
tgtatacaagg

SEQ ID NO: 70 - DNA, 11 bp, Homo sapiens
ttgccttttta

SEQ ID NO: 71 - DNA, 11 bp, Homo sapiens
ttgtgggatct

SEQ ID NO: 72 - DNA, 22 bp, Homo sapiens
agccctggagtaccgccgggct

SEQ ID NO: 73 - DNA, 22 bp, Homo sapiens
ttgccggttaaactggaaggag

SEQ ID NO: 74 - DNA, 22 bp, Homo sapiens
cagagttgtataaatgcgaaca

SEQ ID NO: 75 - DNA, 22 bp, Homo sapiens
gctctgccctcgctctgccccc

SEQ ID NO: 76 - DNA, 33 bp, Homo sapiens
tctttgtctaaggatatatccaatagtgcttcg

SEQ ID NO: 77 - DNA, 11 bp, Homo sapiens
agcctacaggt

SEQ ID NO: 78 - DNA, 11 bp, Homo sapiens
atccacccgcc

SEQ ID NO: 79 - DNA, 11 bp, Homo sapiens
ccagaactctt

SEQ ID NO: 80 - DNA, 11 bp, Homo sapiens
ccctgaagagc

SEQ ID NO: 81 - DNA, 11 bp, Homo sapiens
cgcccgtcgtg

SEQ ID NO: 82 - DNA, 11 bp, Homo sapiens
ctgggatcatc

SEQ ID NO: 83 - DNA, 11 bp, Homo sapiens
gccacagccag

SEQ ID NO: 84 - DNA, 11 bp, Homo sapiens
tgccgtgcctg

SEQ ID NO: 85 - DNA, 11 bp, Homo sapiens
tgtcgggaaat

SEQ ID NO: 86 - DNA, 11 bp, Homo sapiens
ccacaacctgg

SEQ ID NO: 87 - DNA, 33 bp, Homo sapiens
gccgccgagcccccccaatgttcccccaatgct

SEQ ID NO: 88 - DNA, 22 bp, Homo sapiens
gcttacctttccacttgaaaag

SEQ ID NO: 89 - DNA, 44 bp, Homo sapiens
tgttagcctgttataggccgaagtctagaatctctgccatagat

SEQ ID NO: 90 - DNA, 11 bp, Homo sapiens
cctgtacccca

SEQ ID NO: 91 - DNA, 11 bp, Homo sapiens
cggagtccatt

SEQ ID NO: 92 - DNA, 11 bp, Homo sapiens
gagcagcgccc

SEQ ID NO: 93 - DNA, 11 bp, Homo sapiens
gtagcagggct

SEQ ID NO: 94 - DNA, 11 bp, Homo sapiens
tgaggggtgaa

SEQ ID NO: 95 - DNA, 22 bp, Homo sapiens
aagtcattcagaggctggacga

SEQ ID NO: 96 - DNA, 22 bp, Homo sapiens
gtgtgagtgtgatgagctggaa

SEQ ID NO: 97 - DNA, 11 bp, Homo sapiens
cgcattaaagc

SEQ ID NO: 98 - DNA, 11 bp, Homo sapiens
ctcggccagag

SEQ ID NO: 99 - DNA, 11 bp, Homo sapiens
caagcaggaca

SEQ ID NO: 100 - DNA, 11 bp, Homo sapiens
tgatgtctggt

SEQ ID NO: 101 - DNA, 22 bp, Homo sapiens
ttcttattttagtggctgagca

SEQ ID NO: 102 - DNA, 22 bp, Homo sapiens
caggagaactgagtgaggatag

SEQ ID NO: 103 - DNA, 11 bp, Homo sapiens
cagcttgcaaa

SEQ ID NO: 104 - DNA, 11 bp, Homo sapiens
gtttatggata

SEQ ID NO: 105 - DNA, 11 bp, Homo sapiens
gtttgcaagtg

SEQ ID NO: 106 - DNA, 77 bp, Homo sapiens
ttactaaatggtaacagttgtgcgggatgcagacctcactttttcctcactttctacatatactgtaagcaaactaa

SEQ ID NO: 107 - DNA, 44 bp, Homo sapiens
tacaaaaccatgtttttgcttcgaagacggtgaataaaacattc

SEQ ID NO: 108 - DNA, 11 bp, Homo sapiens
aggctttatgg

SEQ ID NO: 109 - DNA, 33 bp, Homo sapiens
ttcagtgaaggtcttgtgtatatcttgtgcata

SEQ ID NO: 110 - DNA, 22 bp, Homo sapiens
cctgtgcctggcctggtcaaga

SEQ ID NO: 111 - DNA, 11 bp, Homo sapiens
cgttcctgcgg

SEQ ID NO: 112 - DNA, 11 bp, Homo sapiens
tgagggaataa

SEQ ID NO: 113 - DNA, 66 bp, Homo sapiens
ttgaatgaacattaaacctcaagatacaaaaaccaactttagggaaatgatacaaaaagtggacct

SEQ ID NO: 114 - DNA, 11 bp, Homo sapiens
gtgccctgttg

SEQ ID NO: 115 - DNA, 11 bp, Homo sapiens
cacgcaatgct

SEQ ID NO: 116 - DNA, 22 bp, Homo sapiens
tatgcccgaatcaggagtgtgc

SEQ ID NO: 117 - DNA, 11 bp, Homo sapiens
tgaccccacag

SEQ ID NO: 118 - DNA, 11 bp, Homo sapiens
tttggggctgg

SEQ ID NO: 119 - DNA, 11 bp, Homo sapiens
gcgggagggct

SEQ ID NO: 120 - DNA, 11 bp, Homo sapiens
tgtgggtgctg

SEQ ID NO: 121 - DNA, 11 bp, Homo sapiens
ctgtgacacag

SEQ ID NO: 122 - DNA, 11 bp, Homo sapiens
ggctttggagt

SEQ ID NO: 123 - DNA, 11 bp, Homo sapiens
agaatcgcttg

SEQ ID NO: 124 - DNA, 11 bp, Homo sapiens
ccctgggttct

SEQ ID NO: 125 - DNA, 11 bp, Homo sapiens
gtgaaacctcg

SEQ ID NO: 126 - DNA, 11 bp, Homo sapiens
ttacgaggaag

SEQ ID NO: 127 - DNA, 22 bp, Homo sapiens
cagcgcctggcaactcccagtt

SEQ ID NO: 128 - DNA, 11 bp, Homo sapiens
aggtgcagagg

SEQ ID NO: 129 - DNA, 11 bp, Homo sapiens
atgtactaaag

SEQ ID NO: 130 - DNA, 11 bp, Homo sapiens
gacgcagaagt

SEQ ID NO: 131 - DNA, 11 bp, Homo sapiens
gagcagctgga

SEQ ID NO: 132 - DNA, 11 bp, Homo sapiens
gggatcgcccc

SEQ ID NO: 133 - DNA, 11 bp, Homo sapiens
gtttcttccct

SEQ ID NO: 134 - DNA, 44 bp, Homo sapiens
gctaagtatttgcccattttatcttgtatatagatattacagtg

SEQ ID NO: 135 - DNA, 22 bp, Homo sapiens
caaaggctgtgaggggattccc

SEQ ID NO: 136 - DNA, 55 bp, Homo sapiens
taaatgatcaggtgtaaccccggtgcgtgctgcgcctgggctccccaggccctgg

SEQ ID NO: 137 - DNA, 11 bp, Homo sapiens
ttgtcgatggg

SEQ ID NO: 138 - DNA, 11 bp, Homo sapiens
gtggcgcacac

SEQ ID NO: 139 - DNA, 11 bp, Homo sapiens
tcagccgctac

SEQ ID NO: 140 - DNA, 11 bp, Homo sapiens
acccgccgggc

SEQ ID NO: 141 - DNA, 11 bp, Homo sapiens
tactgctcgga

SEQ ID NO: 142 - DNA, 11 bp, Homo sapiens
actatagagac

SEQ ID NO: 143 - DNA, 11 bp, Homo sapiens
ccctggtgggc

SEQ ID NO: 144 - DNA, 11 bp, Homo sapiens
ccgcacctcca

SEQ ID NO: 145 - DNA, 11 bp, Homo sapiens
caggcgtgcac

SEQ ID NO: 146 - DNA, 11 bp, Homo sapiens
tcagtattcta

SEQ ID NO: 147 - DNA, 11 bp, Homo sapiens
tcgaggacaga

SEQ ID NO: 148 - DNA, 11 bp, Homo sapiens
aagtgagatgg

SEQ ID NO: 149 - DNA, 11 bp, Homo sapiens
gaattgaaata

SEQ ID NO: 150 - DNA, 11 bp, Homo sapiens
gcagaaccatt

SEQ ID NO: 151 - DNA, 11 bp, Homo sapiens
tacacccgctc

SEQ ID NO: 152 - DNA, 11 bp, Homo sapiens
tggccagatgc

SEQ ID NO: 153 - DNA, 11 bp, Homo sapiens
atccgccactc

SEQ ID NO: 154 - DNA, 11 bp, Homo sapiens
tcccagagacc

SEQ ID NO: 155 - DNA, 11 bp, Homo sapiens
ggaggcaggtg

SEQ ID NO: 156 - DNA, 11 bp, Homo sapiens
gagagaagagt

SEQ ID NO: 157 - DNA, 11 bp, Homo sapiens
aggaacacaaa

SEQ ID NO: 158 - DNA, 11 bp, Homo sapiens
ggcctacatcc

SEQ ID NO: 159 - DNA, 11 bp, Homo sapiens
tgcttgtccct

SEQ ID NO: 160 - DNA, 11 bp, Homo sapiens
tggcaaacgtg

SEQ ID NO: 161 - DNA, 11 bp, Homo sapiens
aggactttgcc

SEQ ID NO: 162 - DNA, 11 bp, Homo sapiens
cccagcaagag

SEQ ID NO: 163 - DNA, 11 bp, Homo sapiens
ctgttacaggt

SEQ ID NO: 164 - DNA, 11 bp, Homo sapiens
ttaataaaata

SEQ ID NO: 165 - DNA, 11 bp, Homo sapiens
ttacaaaggca

SEQ ID NO: 166 - DNA, 11 bp, Homo sapiens
tttgagacctg

SEQ ID NO: 167 - DNA, 11 bp, Homo sapiens
gtaatgtccat

SEQ ID NO: 168 - DNA, 11 bp, Homo sapiens
gccaacggcgt

SEQ ID NO: 169 - DNA, 11 bp, Homo sapiens
tggcctgccca

SEQ ID NO: 170 - DNA, 11 bp, Homo sapiens
gacacgaacaa

SEQ ID NO: 171 - DNA, 11 bp, Homo sapiens
ctcggtgatgt

SEQ ID NO: 172 - DNA, 11 bp, Homo sapiens
atatctttgct

SEQ ID NO: 173 - DNA, 11 bp, Homo sapiens
ctgaagctaag

SEQ ID NO: 174 - DNA, 11 bp, Homo sapiens
gcgaaacccag

SEQ ID NO: 175 - DNA, 11 bp, Homo sapiens
gtttgcaagtg

SEQ ID NO: 176 - DNA, 11 bp, Homo sapiens
ggggtgctgtg

SEQ ID NO: 177 - DNA, 11 bp, Homo sapiens
tggagactggc

SEQ ID NO: 178 - DNA, 11 bp, Homo sapiens
cctccctgatg

SEQ ID NO: 179 - DNA, 11 bp, Homo sapiens
atgtataattt

SEQ ID NO: 180 - DNA, 11 bp, Homo sapiens
ttggctaggcc

SEQ ID NO: 181 - DNA, 11 bp, Homo sapiens
cttgacacaca

SEQ ID NO: 182 - DNA, 11 bp, Homo sapiens
ttcagggcttc

SEQ ID NO: 183 - DNA, 11 bp, Homo sapiens
ggcaggagtag

SEQ ID NO: 184 - DNA, 11 bp, Homo sapiens
aatgagcaact

SEQ ID NO: 185 - DNA, 11 bp, Homo sapiens
gcttaatgtgt

SEQ ID NO: 186 - DNA, 11 bp, Homo sapiens
gcagctatgtg

SEQ ID NO: 187 - DNA, 11 bp, Homo sapiens
agtgccgtgtg

SEQ ID NO: 188 - DNA, 11 bp, Homo sapiens
cggagtccatt

SEQ ID NO: 189 - DNA, 11 bp, Homo sapiens
caagccttact

SEQ ID NO: 190 - DNA, 11 bp, Homo sapiens
tggcccgacga

SEQ ID NO: 191 - DNA, 11 bp, Homo sapiens
atcccttcccg

SEQ ID NO: 192 - DNA, 11 bp, Homo sapiens
gggcacaatgc

SEQ ID NO: 193 - DNA, 11 bp, Homo sapiens
gctaaggagat

SEQ ID NO: 194 - DNA, 11 bp, Homo sapiens
tatgacttaat

SEQ ID NO: 195 - DNA, 11 bp, Homo sapiens
gtttaatagaa

SEQ ID NO: 196 - DNA, 11 bp, Homo sapiens
tgatattccaa

SEQ ID NO: 197 - DNA, 11 bp, Homo sapiens
aactgtactac

SEQ ID NO: 198 - DNA, 11 bp, Homo sapiens
gtcactctccc

SEQ ID NO: 199 - DNA, 11 bp, Homo sapiens
atccgccactc

SEQ ID NO: 200 - DNA, 11 bp, Homo sapiens
tcccagagacc

SEQ ID NO: 201 - DNA, 11 bp, Homo sapiens
ggaggcaggtg

SEQ ID NO: 202 - DNA, 11 bp, Homo sapiens
gagagaagagt

SEQ ID NO: 203 - DNA, 11 bp, Homo sapiens
ccatttaaagc

SEQ ID NO: 204 - DNA, 11 bp, Homo sapiens
gctgggaaccc

SEQ ID NO: 205 - DNA, 11 bp, Homo sapiens
ctcaaaatcaa

SEQ ID NO: 206 - DNA, 11 bp, Homo sapiens
gggaaaccccg

SEQ ID NO: 207 - DNA, 11 bp, Homo sapiens
ggggaaacccc

SEQ ID NO: 208 - DNA, 11 bp, Homo sapiens
tgtctgcctga

SEQ ID NO: 209 - DNA, 11 bp, Homo sapiens
cgcctgtagtc

SEQ ID NO: 210 - DNA, 11 bp, Homo sapiens
gaccttaatgg

SEQ ID NO: 211 - DNA, 11 bp, Homo sapiens
ggacctgcgcc

SEQ ID NO: 212 - DNA, 11 bp, Homo sapiens
atacagccact

SEQ ID NO: 213 - DNA, 11 bp, Homo sapiens
gaccgcaggag

SEQ ID NO: 214 - DNA, 11 bp, Homo sapiens
aagaacctgtg

SEQ ID NO: 215 - DNA, 11 bp, Homo sapiens
gtgtcagtttt

SEQ ID NO: 216 - DNA, 11 bp, Homo sapiens
ttctcaccacc

SEQ ID NO: 217 - DNA, 11 bp, Homo sapiens
gggcggagctc

SEQ ID NO: 218 - DNA, 11 bp, Homo sapiens
gtgctgaatgg

SEQ ID NO: 219 - DNA, 11 bp, Homo sapiens
ggagtgtgctc

SEQ ID NO: 220 - DNA, 11 bp, Homo sapiens
cccttagcttt

SEQ ID NO: 221 - DNA, 11 bp, Homo sapiens
actatttccac

SEQ ID NO: 222 - DNA, 11 bp, Homo sapiens
atccgcctgct

SEQ ID NO: 223 - DNA, 11 bp, Homo sapiens
tagaaaaataa

SEQ ID NO: 224 - DNA, 11 bp, Homo sapiens
ttcatctcttg

SEQ ID NO: 225 - DNA, 11 bp, Homo sapiens
tcctcgggcag

SEQ ID NO: 226 - DNA, 11 bp, Homo sapiens
tgagggaataa

SEQ ID NO: 227 - DNA, 11 bp, Homo sapiens
gagctccacag

SEQ ID NO: 228 - DNA, 11 bp, Homo sapiens
tttcctctcaa

SEQ ID NO: 229 - DNA, 11 bp, Homo sapiens
caacattcctg

SEQ ID NO: 230 - DNA, 11 bp, Homo sapiens
gtgcagctggc

SEQ ID NO: 231 - DNA, 11 bp, Homo sapiens
cctggtcaaga

SEQ ID NO: 232 - DNA, 11 bp, Homo sapiens
gagaaagagga

SEQ ID NO: 233 - DNA, 11 bp, Homo sapiens
ttggctgggct

SEQ ID NO: 234 - DNA, 11 bp, Homo sapiens
aaatatatttt

SEQ ID NO: 235 - DNA, 11 bp, Homo sapiens
cactataaaaa

SEQ ID NO: 236 - DNA, 11 bp, Homo sapiens
ttttatactgc

SEQ ID NO: 237 - DNA, 11 bp, Homo sapiens
ctttgatcagg

SEQ ID NO: 238 - DNA, 11 bp, Homo sapiens
accacaggggc

SEQ ID NO: 239 - DNA, 11 bp, Homo sapiens
accacaggggt

SEQ ID NO: 240 - DNA, 11 bp, Homo sapiens
tttgagttctg

SEQ ID NO: 241 - DNA, 11 bp, Homo sapiens
tctgatatggt

SEQ ID NO: 242 - DNA, 11 bp, Homo sapiens
cggcccatctg

SEQ ID NO: 243 - DNA, 11 bp, Homo sapiens
tttattcctct

SEQ ID NO: 244 - DNA, 11 bp, Homo sapiens
tgatgatcatt

SEQ ID NO: 245 - DNA, 11 bp, Homo sapiens
gtttgcggagg

SEQ ID NO: 246 - DNA, 11 bp, Homo sapiens
ggctttgattt

SEQ ID NO: 247 - DNA, 11 bp, Homo sapiens
aatgtttgtga

SEQ ID NO: 248 - DNA, 11 bp, Homo sapiens
atcgatcgcct

SEQ ID NO: 249 - DNA, 11 bp, Homo sapiens
gggcctggcct

SEQ ID NO: 250 - DNA, 11 bp, Homo sapiens
gcgaagccctg

SEQ ID NO: 251 - DNA, 11 bp, Homo sapiens
gagaccctgga

SEQ ID NO: 252 - DNA, 11 bp, Homo sapiens
cagcaggggat

SEQ ID NO: 253 - DNA, 11 bp, Homo sapiens
ctcttaatgta

SEQ ID NO: 254 - DNA, 11 bp, Homo sapiens
ggccatctctt

SEQ ID NO: 255 - DNA, 11 bp, Homo sapiens
tgaaagggtgt

SEQ ID NO: 256 - DNA, 11 bp, Homo sapiens
tgagagggtgt

SEQ ID NO: 257 - DNA, 11 bp, Homo sapiens
aagaaccagcg

SEQ ID NO: 258 - DNA, 11 bp, Homo sapiens
taacccactgg

SEQ ID NO: 259 - DNA, 11 bp, Homo sapiens
ccgggtgatgg

SEQ ID NO: 260 - DNA, 11 bp, Homo sapiens
cccgggagcga

SEQ ID NO: 261 - DNA, 11 bp, Homo sapiens
gatgccggcac

SEQ ID NO: 262 - DNA, 11 bp, Homo sapiens
gcttaatgttt

SEQ ID NO: 263 - DNA, 11 bp, Homo sapiens
cttgacatacc

SEQ ID NO: 264 - DNA, 11 bp, Homo sapiens
ggtgtgagcca

SEQ ID NO: 265 - DNA, 11 bp, Homo sapiens
aaccctgcccc

SEQ ID NO: 266 - DNA, 11 bp, Homo sapiens
gtgggcctttg

SEQ ID NO: 267 - DNA, 11 bp, Homo sapiens
tggcccgacga

SEQ ID NO: 268 - DNA, 11 bp, Homo sapiens
tgacagtgact

SEQ ID NO: 269 - DNA, 11 bp, Homo sapiens
actgccccact

SEQ ID NO: 270 - DNA, 11 bp, Homo sapiens
ttttcttcatt

SEQ ID NO: 271 - DNA, 11 bp, Homo sapiens
cctccacctag

SEQ ID NO: 272 - DNA, 11 bp, Homo sapiens
gtggtacagga

SEQ ID NO: 273 - DNA, 11 bp, Homo sapiens
gtggtgtgtac

SEQ ID NO: 274 - DNA, 11 bp, Homo sapiens
taactctcctg

SEQ ID NO: 275 - DNA, 11 bp, Homo sapiens
aataaagcctt

SEQ ID NO: 276 - DNA, 11 bp, Homo sapiens
gagaaatctac

SEQ ID NO: 277 - DNA, 11 bp, Homo sapiens
tctttgttgtt

SEQ ID NO: 278 - DNA, 11 bp, Homo sapiens
tgtgatagtaa

SEQ ID NO: 279 - DNA, 11 bp, Homo sapiens
atggccataga

SEQ ID NO: 280 - DNA, 11 bp, Homo sapiens
aaaaagcagat

SEQ ID NO: 281 - DNA, 11 bp, Homo sapiens
acatttcctgt

SEQ ID NO: 282 - DNA, 11 bp, Homo sapiens
caggccttcag

SEQ ID NO: 283 - DNA, 11 bp, Homo sapiens
gcttgcaaaaa

SEQ ID NO: 284 - DNA, 11 bp, Homo sapiens
attaacaaagc

SEQ ID NO: 285 - DNA, 11 bp, Homo sapiens
aagcaaactaa

SEQ ID NO: 286 - DNA, 11 bp, Homo sapiens
cctcactttct

SEQ ID NO: 287 - DNA, 11 bp, Homo sapiens
cctcacttttt

SEQ ID NO: 288 - DNA, 11 bp, Homo sapiens
cgggatgcaga

SEQ ID NO: 289 - DNA, 11 bp, Homo sapiens
taacagttgtg

SEQ ID NO: 290 - DNA, 11 bp, Homo sapiens
ttactaaatgg

SEQ ID NO: 291 - DNA, 11 bp, Homo sapiens
gtggaataaag

SEQ ID NO: 292 - DNA, 11 bp, Homo sapiens
gcgaaaccctg

SEQ ID NO: 293 - DNA, 11 bp, Homo sapiens
aagtgaaacac

SEQ ID NO: 294 - DNA, 11 bp, Homo sapiens
atccagggtcc

SEQ ID NO: 295 - DNA, 11 bp, Homo sapiens
gacacttgggg

SEQ ID NO: 296 - DNA, 11 bp, Homo sapiens
gttctcccact

SEQ ID NO: 297 - DNA, 11 bp, Homo sapiens
tttatgtctgg

SEQ ID NO: 298 - DNA, 11 bp, Homo sapiens
cagaaaaaagc

SEQ ID NO: 299 - DNA, 11 bp, Homo sapiens
cttcaggacct

SEQ ID NO: 300 - DNA, 11 bp, Homo sapiens
tcagagatgag

SEQ ID NO: 301 - DNA, 11 bp, Homo sapiens
aacattctaag

SEQ ID NO: 302 - DNA, 11 bp, Homo sapiens
ggaatacagaa

SEQ ID NO: 303 - DNA, 11 bp, Homo sapiens
tctggactttt

SEQ ID NO: 304 - DNA, 11 bp, Homo sapiens
ctgctaagatg

SEQ ID NO: 305 - DNA, 11 bp, Homo sapiens
tatgaccacaa

SEQ ID NO: 306 - DNA, 11 bp, Homo sapiens
aatacaggatc

SEQ ID NO: 307 - DNA, 11 bp, Homo sapiens
gctggagctag

SEQ ID NO: 308 - DNA, 11 bp, Homo sapiens
gcatcttcaat

SEQ ID NO: 309 - DNA, 11 bp, Homo sapiens
ctgctgcactc

SEQ ID NO: 310 - DNA, 11 bp, Homo sapiens
agacgcactct

SEQ ID NO: 311 - DNA, 11 bp, Homo sapiens
ttagacattac

SEQ ID NO: 312 - DNA, 11 bp, Homo sapiens
ccgtttagcag

SEQ ID NO: 313 - DNA, 11 bp, Homo sapiens
tcataactgtc

SEQ ID NO: 314 - DNA, 11 bp, Homo sapiens
ggttccctgag

SEQ ID NO: 315 - DNA, 11 bp, Homo sapiens
tccgagccccc

SEQ ID NO: 316 - DNA, 11 bp, Homo sapiens
aattaattgta

SEQ ID NO: 317 - DNA, 11 bp, Homo sapiens
aggctttaggg

SEQ ID NO: 318 - DNA, 11 bp, Homo sapiens
ggagaagatga

SEQ ID NO: 319 - DNA, 11 bp, Homo sapiens
gaaatgatgag

SEQ ID NO: 320 - DNA, 11 bp, Homo sapiens
ttgctagaggg

SEQ ID NO: 321 - DNA, 11 bp, Homo sapiens
aaattaaaaca

SEQ ID NO: 322 - DNA, 11 bp, Homo sapiens
gcctgtttggg

SEQ ID NO: 323 - DNA, 11 bp, Homo sapiens
ctaaaatgctt

SEQ ID NO: 324 - DNA, 11 bp, Homo sapiens
gaaaaagatgt

SEQ ID NO: 325 - DNA, 11 bp, Homo sapiens
ggaaatattcc

SEQ ID NO: 326 - DNA, 11 bp, Homo sapiens
agtgaggatag

SEQ ID NO: 327 - DNA, 11 bp, Homo sapiens
caggagaactg

SEQ ID NO: 328 - DNA, 11 bp, Homo sapiens
gggctgctgcc

SEQ ID NO: 329 - DNA, 11 bp, Homo sapiens
gagactgtagg

SEQ ID NO: 330 - DNA, 11 bp, Homo sapiens
tgaacccgcca

SEQ ID NO: 331 - DNA, 11 bp, Homo sapiens
aaaactgggga

SEQ ID NO: 332 - DNA, 11 bp, Homo sapiens
cccccaattct

SEQ ID NO: 333 - DNA, 11 bp, Homo sapiens
aagaaccagcg

SEQ ID NO: 334 - DNA, 11 bp, Homo sapiens
taacccactgg

SEQ ID NO: 335 - DNA, 11 bp, Homo sapiens
cagctgagggc

SEQ ID NO: 336 - DNA, 11 bp, Homo sapiens
cgcacacacat

SEQ ID NO: 337 - DNA, 11 bp, Homo sapiens
agggcaaggcc

SEQ ID NO: 338 - DNA, 11 bp, Homo sapiens
tttacagctgg

SEQ ID NO: 339 - DNA, 11 bp, Homo sapiens
ctttaaaatat

SEQ ID NO: 340 - DNA, 11 bp, Homo sapiens
ggggactggtg

SEQ ID NO: 341 - DNA, 11 bp, Homo sapiens
gtacttcctct

SEQ ID NO: 342 - DNA, 11 bp, Homo sapiens
tcagtgaccag

SEQ ID NO: 343 - DNA, 11 bp, Homo sapiens
tgaaaacctga

SEQ ID NO: 344 - DNA, 11 bp, Homo sapiens
cggtttccaag

SEQ ID NO: 345 - DNA, 11 bp, Homo sapiens
gccttgatctc

SEQ ID NO: 346 - DNA, 11 bp, Homo sapiens
tggattttggg

SEQ ID NO: 347 - DNA, 11 bp, Homo sapiens
tgtatacaagg

SEQ ID NO: 348 - DNA, 11 bp, Homo sapiens
ggcctgggggt

SEQ ID NO: 349 - DNA, 11 bp, Homo sapiens
gttgctgtccc

SEQ ID NO: 350 - DNA, 11 bp, Homo sapiens
tatgagcacga

SEQ ID NO: 351 - DNA, 11 bp, Homo sapiens
acatcatactg

SEQ ID NO: 352 - DNA, 11 bp, Homo sapiens
ttctctccaca

SEQ ID NO: 353 - DNA, 11 bp, Homo sapiens
gtttatggata

SEQ ID NO: 354 - DNA, 11 bp, Homo sapiens
cctggcagtca

SEQ ID NO: 355 - DNA, 11 bp, Homo sapiens
atctgtcactt

SEQ ID NO: 356 - DNA, 11 bp, Homo sapiens
aagccttgctg

SEQ ID NO: 357 - DNA, 11 bp, Homo sapiens
atgtcaaccaa

SEQ ID NO: 358 - DNA, 11 bp, Homo sapiens
tctgtggctca

SEQ ID NO: 359 - DNA, 11 bp, Homo sapiens
ttgaactggcc

SEQ ID NO: 360 - DNA, 11 bp, Homo sapiens
attaggaactg

SEQ ID NO: 361 - DNA, 11 bp, Homo sapiens
gacaaagcaag

SEQ ID NO: 362 - DNA, 11 bp, Homo sapiens
ctgacccagcc

SEQ ID NO: 363 - DNA, 11 bp, Homo sapiens
gtgcaaaatgg

SEQ ID NO: 364 - DNA, 11 bp, Homo sapiens
ctcaggagaga

SEQ ID NO: 365 - DNA, 11 bp, Homo sapiens
gcctgctccct

SEQ ID NO: 366 - DNA, 11 bp, Homo sapiens
tatacgttatg

SEQ ID NO: 367 - DNA, 11 bp, Homo sapiens
ggctgcagtct

SEQ ID NO: 368 - DNA, 11 bp, Homo sapiens
cctggcagtca

SEQ ID NO: 369 - DNA, 11 bp, Homo sapiens
atctgtcactt

SEQ ID NO: 370 - DNA, 11 bp, Homo sapiens
aagccttgctg

SEQ ID NO: 371 - DNA, 11 bp, Homo sapiens
atgtcaaccaa

SEQ ID NO: 372 - DNA, 11 bp, Homo sapiens
tctgtggctca

SEQ ID NO: 373 - DNA, 11 bp, Homo sapiens
ttgaactggcc

SEQ ID NO: 374 - DNA, 11 bp, Homo sapiens
attaggaactg

SEQ ID NO: 375 - DNA, 11 bp, Homo sapiens
gacaaagcaag

SEQ ID NO: 376 - DNA, 11 bp, Homo sapiens
ctgacccagcc

SEQ ID NO: 377 - DNA, 11 bp, Homo sapiens
gtgcaaaatgg

SEQ ID NO: 378 - DNA, 11 bp, Homo sapiens
ctcaggagaga

SEQ ID NO: 379 - DNA, 11 bp, Homo sapiens
gcctgctccct

SEQ ID NO: 380 - DNA, 11 bp, Homo sapiens
tatacgttatg

SEQ ID NO: 381 - DNA, 11 bp, Homo sapiens
ggctgcagtct

SEQ ID NO: 382 - DNA, 11 bp, Homo sapiens
gaccgcaggag

SEQ ID NO: 383 - DNA, 11 bp, Homo sapiens
aagaacctgtg

SEQ ID NO: 384 - DNA, 11 bp, Homo sapiens
gtgtcagtttt

SEQ ID NO: 385 - DNA, 11 bp, Homo sapiens
gttgtcatcac

SEQ ID NO: 386 - DNA, 11 bp, Homo sapiens
tgtgggtgctg

SEQ ID NO: 387 - DNA, 11 bp, Homo sapiens
cctagacctgg

SEQ ID NO: 388 - DNA, 11 bp, Homo sapiens
agcacccaccc

SEQ ID NO: 389 - DNA, 11 bp, Homo sapiens
gcctcagcctc

SEQ ID NO: 390 - DNA, 11 bp, Homo sapiens
caggagtgtgc

SEQ ID NO: 391 - DNA, 11 bp, Homo sapiens
tatgcccgaat

SEQ ID NO: 392 - DNA, 11 bp, Homo sapiens
taactggcctt

SEQ ID NO: 393 - DNA, 11 bp, Homo sapiens
ccatttaaagc

SEQ ID NO: 394 - DNA, 11 bp, Homo sapiens
gctgggaaccc

SEQ ID NO: 395 - DNA, 11 bp, Homo sapiens
ctcaaaatcaa

SEQ ID NO: 396 - DNA, 11 bp, Homo sapiens
gggaaaccccg

SEQ ID NO: 397 - DNA, 11 bp, Homo sapiens
ggggaaacccc

SEQ ID NO: 398 - DNA, 11 bp, Homo sapiens
tgtctgcctga

SEQ ID NO: 399 - DNA, 11 bp, Homo sapiens
gggctgctctt

SEQ ID NO: 400 - DNA, 11 bp, Homo sapiens
cgacagcgttc

SEQ ID NO: 401 - DNA, 11 bp, Homo sapiens
tcctatcccag

SEQ ID NO: 402 - DNA, 11 bp, Homo sapiens
gaagtacagta

SEQ ID NO: 403 - DNA, 11 bp, Homo sapiens
gacagatggac

SEQ ID NO: 404 - DNA, 11 bp, Homo sapiens
aagtgaggaga

SEQ ID NO: 405 - DNA, 11 bp, Homo sapiens
tgccgtgcctg

SEQ ID NO: 406 - DNA, 11 bp, Homo sapiens
taaaagatgta

SEQ ID NO: 407 - DNA, 11 bp, Homo sapiens
ttacactgtaa

SEQ ID NO: 408 - DNA, 11 bp, Homo sapiens
attgtacaaca

SEQ ID NO: 409 - DNA, 11 bp, Homo sapiens
gcctcttgaag

SEQ ID NO: 410 - DNA, 11 bp, Homo sapiens
gccaacggcgt

SEQ ID NO: 411 - DNA, 11 bp, Homo sapiens
tggcctgccca

SEQ ID NO: 412 - DNA, 11 bp, Homo sapiens
cttggtaattt

SEQ ID NO: 413 - DNA, 11 bp, Homo sapiens
ttgcctgcagt

SEQ ID NO: 414 - DNA, 11 bp, Homo sapiens
agtgtatcaca

SEQ ID NO: 415 - DNA, 11 bp, Homo sapiens
cggagtccatt

SEQ ID NO: 416 - DNA, 11 bp, Homo sapiens
atcccttcccg

SEQ ID NO: 417 - DNA, 11 bp, Homo sapiens
gggcacaatgc

SEQ ID NO: 418 - DNA, 11 bp, Homo sapiens
tggctgttaat

SEQ ID NO: 419 - DNA, 11 bp, Homo sapiens
agctgatcagc

SEQ ID NO: 420 - DNA, 11 bp, Homo sapiens
ccatttaaagc

SEQ ID NO: 421 - DNA, 11 bp, Homo sapiens
gctgggaaccc

SEQ ID NO: 422 - DNA, 11 bp, Homo sapiens
ctcaaaatcaa

SEQ ID NO: 423 - DNA, 11 bp, Homo sapiens
gggaaaccccg

SEQ ID NO: 424 - DNA, 11 bp, Homo sapiens
ggggaaacccc

SEQ ID NO: 425 - DNA, 11 bp, Homo sapiens
tgtctgcctga

SEQ ID NO: 426 - DNA, 11 bp, Homo sapiens
gagaagacttc

SEQ ID NO: 427 - DNA, 11 bp, Homo sapiens
atttttggtgg

SEQ ID NO: 428 - DNA, 11 bp, Homo sapiens
actcttaatgt

SEQ ID NO: 429 - DNA, 11 bp, Homo sapiens
atgcccgcacc

SEQ ID NO: 430 - DNA, 11 bp, Homo sapiens
gacaggtaaag

SEQ ID NO: 431 - DNA, 11 bp, Homo sapiens
gattttttttc

SEQ ID NO: 432 - DNA, 11 bp, Homo sapiens
tcagatctttg

SEQ ID NO: 433 - DNA, 11 bp, Homo sapiens
tcagatttttg

SEQ ID NO: 434 - DNA, 11 bp, Homo sapiens
gagagctacat

SEQ ID NO: 435 - DNA, 11 bp, Homo sapiens
gcgatggccgt

SEQ ID NO: 436 - DNA, 11 bp, Homo sapiens
gtctgccctct

SEQ ID NO: 437 - DNA, 11 bp, Homo sapiens
gtgctggtgct

SEQ ID NO: 438 - DNA, 11 bp, Homo sapiens
gatgccggcac

SEQ ID NO: 439 - DNA, 11 bp, Homo sapiens
ctcattcggcc

SEQ ID NO: 440 - DNA, 11 bp, Homo sapiens
gagattgaggg

SEQ ID NO: 441 - DNA, 11 bp, Homo sapiens
aaagtggaaac

SEQ ID NO: 442 - DNA, 11 bp, Homo sapiens
ctgaggtgatg

SEQ ID NO: 443 - DNA, 11 bp, Homo sapiens
tcgggtgtggg

SEQ ID NO: 444 - DNA, 11 bp, Homo sapiens
caatgtcttca

SEQ ID NO: 445 - DNA, 11 bp, Homo sapiens
cctcaggctcc

SEQ ID NO: 446 - DNA, 11 bp, Homo sapiens
gacaccagggt

SEQ ID NO: 447 - DNA, 11 bp, Homo sapiens
ttaataaatgt

SEQ ID NO: 448 - DNA, 11 bp, Homo sapiens
ttggttgcact

SEQ ID NO: 449 - DNA, 11 bp, Homo sapiens
ccccgggcctc

SEQ ID NO: 450 - DNA, 11 bp, Homo sapiens
gagctccacag

SEQ ID NO: 451 - DNA, 11 bp, Homo sapiens
tccccccattc

SEQ ID NO: 452 - DNA, 11 bp, Homo sapiens
ttcagtgggtt

SEQ ID NO: 453 - DNA, 11 bp, Homo sapiens
accaatttaaa

SEQ ID NO: 454 - DNA, 11 bp, Homo sapiens
tgtgctaatat

SEQ ID NO: 455 - DNA, 11 bp, Homo sapiens
gcatagttcta

SEQ ID NO: 456 - DNA, 11 bp, Homo sapiens
agagtcataca

SEQ ID NO: 457 - DNA, 11 bp, Homo sapiens
caggagtgtgc

SEQ ID NO: 458 - DNA, 11 bp, Homo sapiens
tatgcccgaat

SEQ ID NO: 459 - DNA, 11 bp, Homo sapiens
gcatagttcta

SEQ ID NO: 460 - DNA, 11 bp, Homo sapiens
agagtcataca

SEQ ID NO: 461 - DNA, 11 bp, Homo sapiens
ctcccctgccc

SEQ ID NO: 462 - DNA, 11 bp, Homo sapiens
ttcccctgccc

SEQ ID NO: 463 - DNA, 11 bp, Homo sapiens
tcaccggtcag

SEQ ID NO: 464 - DNA, 11 bp, Homo sapiens
ttctctccaca

SEQ ID NO: 465 - DNA, 11 bp, Homo sapiens
gtttatggata

SEQ ID NO: 466 - DNA, 11 bp, Homo sapiens
cagtaaaaaaa

SEQ ID NO: 467 - DNA, 11 bp, Homo sapiens
catttttcccc

SEQ ID NO: 468 - DNA, 11 bp, Homo sapiens
taacaagttct

SEQ ID NO: 469 - DNA, 11 bp, Homo sapiens
tatatcagtgt

SEQ ID NO: 470 - DNA, 11 bp, Homo sapiens
tattacttggt

SEQ ID NO: 471 - DNA, 11 bp, Homo sapiens
ttcactgccga

SEQ ID NO: 472 - DNA, 11 bp, Homo sapiens
cgcagtgtcct

SEQ ID NO: 473 - DNA, 11 bp, Homo sapiens
tttggggctgg

SEQ ID NO: 474 - DNA, 11 bp, Homo sapiens
aatatgcttta

SEQ ID NO: 475 - DNA, 11 bp, Homo sapiens
ggagccattct

SEQ ID NO: 476 - DNA, 11 bp, Homo sapiens
ggaaggacaga

SEQ ID NO: 477 - DNA, 11 bp, Homo sapiens
aaatacagcag

SEQ ID NO: 478 - DNA, 11 bp, Homo sapiens
gccgccatcaa

SEQ ID NO: 479 - DNA, 11 bp, Homo sapiens
tttgcctgtta

SEQ ID NO: 480 - DNA, 11 bp, Homo sapiens
tttttacagtg

SEQ ID NO: 481 - DNA, 11 bp, Homo sapiens
ctctacagtgc

SEQ ID NO: 482 - DNA, 11 bp, Homo sapiens
tggctgtgagg

SEQ ID NO: 483 - DNA, 11 bp, Homo sapiens
gggtgcttggt

SEQ ID NO: 484 - DNA, 11 bp, Homo sapiens
aatgtgatttc

SEQ ID NO: 485 - DNA, 11 bp, Homo sapiens
gcggttgtggc

SEQ ID NO: 486 - DNA, 11 bp, Homo sapiens
caccaggccat

SEQ ID NO: 487 - DNA, 11 bp, Homo sapiens
gtgatgcgcat

SEQ ID NO: 488 - DNA, 11 bp, Homo sapiens
caggttgtgag

SEQ ID NO: 489 - DNA, 11 bp, Homo sapiens
gaaatacagtt

SEQ ID NO: 490 - DNA, 11 bp, Homo sapiens
agctgagctaa

SEQ ID NO: 491 - DNA, 11 bp, Homo sapiens
agaagtgtcct

SEQ ID NO: 492 - DNA, 11 bp, Homo sapiens
gggctctgagc

SEQ ID NO: 493 - DNA, 11 bp, Homo sapiens
tcacttgctgt

SEQ ID NO: 494 - DNA, 11 bp, Homo sapiens
ataatttttaa

SEQ ID NO: 495 - DNA, 11 bp, Homo sapiens
ctcacacatta

SEQ ID NO: 496 - DNA, 11 bp, Homo sapiens
caaataacaag

SEQ ID NO: 497 - DNA, 11 bp, Homo sapiens
caactgcctat

SEQ ID NO: 498 - DNA, 11 bp, Homo sapiens
gccattataag

SEQ ID NO: 499 - DNA, 11 bp, Homo sapiens
ttttttcttca

SEQ ID NO: 500 - DNA, 11 bp, Homo sapiens
caaccatcatc

SEQ ID NO: 501 - DNA, 11 bp, Homo sapiens
tttctagtttg

SEQ ID NO: 502 - DNA, 11 bp, Homo sapiens
actgactatca

SEQ ID NO: 503 - DNA, 11 bp, Homo sapiens
gagtagaggcc

SEQ ID NO: 504 - DNA, 11 bp, Homo sapiens
acgacaaagct

SEQ ID NO: 505 - DNA, 11 bp, Homo sapiens
cagttacttag

SEQ ID NO: 506 - DNA, 11 bp, Homo sapiens
cttttcagcaa

SEQ ID NO: 507 - DNA, 11 bp, Homo sapiens
gaattaacatt

SEQ ID NO: 508 - DNA, 11 bp, Homo sapiens
gcgctgtcagg

SEQ ID NO: 509 - DNA, 11 bp, Homo sapiens
tcaatcaagat

SEQ ID NO: 510 - DNA, 11 bp, Homo sapiens
aatgtgagtca

SEQ ID NO: 511 - DNA, 11 bp, Homo sapiens
tcactatagca

SEQ ID NO: 512 - DNA, 11 bp, Homo sapiens
ctcttaatgta

SEQ ID NO: 513 - DNA, 11 bp, Homo sapiens
ggccatctctt

SEQ ID NO: 514 - DNA, 11 bp, Homo sapiens
tgaaagggtgt

SEQ ID NO: 515 - DNA, 11 bp, Homo sapiens
tgagagggtgt

SEQ ID NO: 516 - DNA, 11 bp, Homo sapiens
atctttctggc

SEQ ID NO: 517 - DNA, 11 bp, Homo sapiens
gccaccaagta

SEQ ID NO: 518 - DNA, 11 bp, Homo sapiens
taagtggaata

SEQ ID NO: 519 - DNA, 11 bp, Homo sapiens
ttaggcaagta

SEQ ID NO: 520 - DNA, 11 bp, Homo sapiens
aatggatgaac

SEQ ID NO: 521 - DNA, 11 bp, Homo sapiens
attaagaggga

SEQ ID NO: 522 - DNA, 11 bp, Homo sapiens
ccagaggctgt

SEQ ID NO: 523 - DNA, 11 bp, Homo sapiens
ccgacgggcgc

SEQ ID NO: 524 - DNA, 11 bp, Homo sapiens
cgcgtcactaa

SEQ ID NO: 525 - DNA, 11 bp, Homo sapiens
ctaactagtta

SEQ ID NO: 526 - DNA, 11 bp, Homo sapiens
gcaacaacaca

SEQ ID NO: 527 - DNA, 11 bp, Homo sapiens
gccgttcttag

SEQ ID NO: 528 - DNA, 11 bp, Homo sapiens
cctgtcatccc

SEQ ID NO: 529 - DNA, 11 bp, Homo sapiens
gaacccttctc

SEQ ID NO: 530 - DNA, 11 bp, Homo sapiens
acccgccgggc

SEQ ID NO: 531 - DNA, 11 bp, Homo sapiens
agaggtgtaga

SEQ ID NO: 532 - DNA, 11 bp, Homo sapiens
gaagtcggaat

SEQ ID NO: 533 - DNA, 11 bp, Homo sapiens
ggtcagtcggt

SEQ ID NO: 534 - DNA, 11 bp, Homo sapiens
gtaatcctgct

SEQ ID NO: 535 - DNA, 11 bp, Homo sapiens
gtgaccacggg

SEQ ID NO: 536 - DNA, 11 bp, Homo sapiens
tggcgtacgga

SEQ ID NO: 537 - DNA, 11 bp, Homo sapiens
ttggaacaatg

SEQ ID NO: 538 - DNA, 11 bp, Homo sapiens
agccaccgcgc

SEQ ID NO: 539 - DNA, 11 bp, Homo sapiens
cctataatccc

SEQ ID NO: 540 - DNA, 11 bp, Homo sapiens
ttggtcaggct

SEQ ID NO: 541 - DNA, 11 bp, Homo sapiens
gtaggcacggc

SEQ ID NO: 542 - DNA, 11 bp, Homo sapiens
taagccctttt

SEQ ID NO: 543 - DNA, 11 bp, Homo sapiens
tgctgtgtgct

SEQ ID NO: 544 - DNA, 11 bp, Homo sapiens
ggcagagggct

SEQ ID NO: 545 - DNA, 11 bp, Homo sapiens
gtttcttccct

SEQ ID NO: 546 - DNA, 11 bp, Homo sapiens
cagttccataa

SEQ ID NO: 547 - DNA, 11 bp, Homo sapiens
ccctgtaataa

SEQ ID NO: 548 - DNA, 11 bp, Homo sapiens
aataaagcctt

SEQ ID NO: 549 - DNA, 11 bp, Homo sapiens
gagaaatctac

SEQ ID NO: 550 - DNA, 11 bp, Homo sapiens
tctttgttgtt

SEQ ID NO: 551 - DNA, 11 bp, Homo sapiens
tgtgatagtaa

SEQ ID NO: 552 - DNA, 11 bp, Homo sapiens
ccttgaccaat

SEQ ID NO: 553 - DNA, 11 bp, Homo sapiens
gtgtggtattc

SEQ ID NO: 554 - DNA, 11 bp, Homo sapiens
tcttccccagt

SEQ ID NO: 555 - DNA, 11 bp, Homo sapiens
ctcggaggcct

SEQ ID NO: 556 - DNA, 11 bp, Homo sapiens
cagttacttag

SEQ ID NO: 557 - DNA, 11 bp, Homo sapiens
cttttcagcaa

SEQ ID NO: 558 - DNA, 11 bp, Homo sapiens
gaattaacatt

SEQ ID NO: 559 - DNA, 11 bp, Homo sapiens
gcgctgtcagg

SEQ ID NO: 560 - DNA, 11 bp, Homo sapiens
tcaatcaagat

SEQ ID NO: 561 - DNA, 11 bp, Homo sapiens
aatgtgagtca

SEQ ID NO: 562 - DNA, 11 bp, Homo sapiens
tcactatagca

SEQ ID NO: 563 - DNA, 11 bp, Homo sapiens
ctcttaatgta

SEQ ID NO: 564 - DNA, 11 bp, Homo sapiens
ggccatctctt

SEQ ID NO: 565 - DNA, 11 bp, Homo sapiens
tgaaagggtgt

SEQ ID NO: 566 - DNA, 11 bp, Homo sapiens
tgagagggtgt

SEQ ID NO: 567 - DNA, 11 bp, Homo sapiens
atctttctggc

SEQ ID NO: 568 - DNA, 11 bp, Homo sapiens
gccaccaagta

SEQ ID NO: 569 - DNA, 11 bp, Homo sapiens
taagtggaata

SEQ ID NO: 570 - DNA, 11 bp, Homo sapiens
ttaggcaagta